(19) United States
(12) Patent Application Publication
(10) Pub. No.: US 2026/0028582 A1
(43) Pub. Date: Jan. 29, 2026

(54) POLYMER COATINGS FOR SOLID SUPPORTS

(71) Applicant: Singular Genomics Systems, Inc., San Diego, CA (US)

(72) Inventors: Hojin KIM, San Diego, CA (US); Mark KALAJ, San Diego, CA (US); Daan WITTERS, San Diego, CA (US)

(21) Appl. No.: 19/282,910

(22) Filed: Jul. 28, 2025

Related U.S. Application Data
(60) Provisional application No. 63/676,565, filed on Jul. 29, 2024.

(51) Int. Cl. C12N 5/00

(52) U.S. Cl. CPC C12N 5/0012; C12N 2533/14; C12N 2533/30; C12N 2537/00; C12N 2539/00

(57) ABSTRACT

Disclosed herein, inter alia, are compositions and methods for making surface coatings for tissue adherence and/or retention.

### CROSS-REFERENCES TO RELATED APPLICATIONS

This application claims the benefit of U.S. Provisional Application No. 63/676,565, filed Jul. 29, 2024, which is incorporated herein by reference in its entirety and for all purposes.

### BACKGROUND

Flow cells are integral components in next-generation sequencing (NGS) technologies, serving as sophisticated platforms for DNA sequencing. At their core, a flow cell is a hollow glass slide with one or more channels (“lanes”), through which reagents and solutions (e.g., polymerases, nucleotides, and buffers) may traverse. A surface within the channel includes a plurality of immobilized oligonucleotides for capturing target nucleic acid fragments of interest. The captured target molecules may then be amplified and sequenced. While flow cells have been instrumental in transforming the efficiency and economic feasibility of NGS, their application in spatial biology, particularly for in situ transcript detection within cells and tissues, remains unexplored and challenging. A primary impediment in this translation is tissue delamination, a process where the structural integrity of tissue samples is compromised during repetitive reagent exchanges and thermal changes. Furthermore, when utilizing tissue sections, commonly around 5-7 μm in thickness, additional complications arise. Thin tissue sections are prone to wrinkling, which leads to non-uniform exposure to reagents and target detection inefficiencies. Degradation can result from both the mechanical handling of these fragile sections and the chemical interactions during the sequencing process, leading to a loss of vital biological information and potential misinterpretation of sequencing data. Disclosed herein, inter alia, are solutions to these and other problems in the art.

### BRIEF SUMMARY

In an aspect is provided a solid support. In embodiments the solid support includes a polymer attached to the solid support; and a cell sample or tissue section attached to the polymer.

In an aspect is provided a flow cell assembly. In embodiments, the flow cell assembly includes a first solid support; a polymer attached to the first solid support; a cell or tissue attached to the polymer; a second solid support attached to the first solid support, wherein the second solid support is configured to define a reaction chamber (e.g., a flow cell channel). In embodiments, the second solid support is configured to define a reaction chamber when attached to the first solid support.

In an aspect is provided a microfluidic device including the flow cell assembly as described herein. In embodiments, the microfluidic device includes a flow cell. In embodiments, the microfluidic device includes a functionalized tissue slide. In embodiments, the microfluidic device includes an imaging system or detection apparatus.

In an aspect is provided a kit, including the flow cell assembly as described herein. In an aspect is provided a kit, including the first solid support attached to a polymer, which is further attached to an IR reflective coating, and/or particles; and a second solid support, configured to define a reaction chamber when attached to the first solid support. Generally, the kit includes one or more containers providing a composition and one or more additional reagents (e.g., a buffer suitable for polynucleotide extension). The kit may also include a template nucleic acid (DNA and/or RNA), one or more primer polynucleotides, nucleoside triphosphates (including, e.g., deoxyribonucleotides, ribonucleotides, particles, polymerases, labeled nucleotides, and/or modified nucleotides), buffers, salts, and/or labels (e.g., fluorophores). In embodiments, the kit includes a plurality of detection agents capable of detecting a biomolecule (or plurality thereof) from a tissue section. In embodiments, kit includes the flow cell assembly as described herein and a flow cell carrier (e.g., a flow cell carrier as described in U.S. Pat. No. 11,747,262, which is incorporated herein by reference for all purposes).

In an aspect is provided a method of making a solid support, the method including binding a polymer to the solid support. In an aspect is provided a method of making a flow cell assembly, the method including: binding a polymer to a first solid support; attaching a cell or tissue to the polymer; and affixing a second solid support to the first solid support, wherein the first solid support or the second solid support includes a port (e.g., an inlet or outlet port). In embodiments, the second solid support includes 2-4 inlet ports and 2-4 outlet ports. In embodiments, the first solid support and second solid support are described herein including embodiments. In embodiments, binding a polymer to a first solid support is as described herein.

In an aspect is provided a method of detecting a biomolecule in or on a cell or tissue, the method including immobilizing a cell or tissue including a biomolecule to a first solid support, wherein the first solid support includes a polymer attached to the first solid support; attaching a second solid support to the first solid support; contacting the biomolecule in or on the cell or tissue with a detection agent including a label; detecting the label, thereby detecting the biomolecule. In embodiments, the method includes imaging the cell or tissue.

### BRIEF DESCRIPTION OF THE DRAWINGS

FIG. 1. An illustration of a non-limiting example of the monomer (top) and resulting polymer (bottom). In embodiments, the monomer includes an acrylate portion and a protected amine portion. During suitable polymerization conditions, for example via an initiator molecule, reactive species (e.g., radical moiety) reacts with the acrylate monomer, creating a new reactive site at the end of the growing polymer chain. The reactive site then reacts with another acrylate monomer, and this process continues, leading to the growth of the polymer chain. As a result of this polymerization process, the acrylate monomers form a polyacrylate polymer (bottom).

FIGS. 2A-2B. The polymer is allowed to contact and bind to a suitable solid support. Illustrated in FIG. 2A is a solid support (e.g., glass slide), coated with a resist polymer (e.g., an organically modified ceramic resist polymer), binds to the polyacrylate portion of the polymer orienting the protected amine moieties away from the surface. Following deprotection, such as incubation with a reducing or cleaving agent, the amine protecting group is released rendering unprotected amines (FIG. 2B).

FIG. 3 shows a comparison of tissue adherence to solid supports including an amine-containing polymer control (PEI) or a poly-PEGMA amine surface. The surfaces of all three solid supports were functionalized with Ormocomp® prior to the deposition of PEI or the poly-PEGMA polymers. Tissue adherence was evaluated for each solid support using lymph, breast, lung, colon, kidney, and tonsil tissues. The blank circles indicate a lack of tissue sample at that location. Following tissue deposition, the tissues are baked at 60° C. for 30 minutes and an image is obtained. The supports were then subjected to an antigen retrieval protocol, including elevating the temperature to 120° C. for 30 minutes in an EDTA pH 9.0 buffer. Following antigen retrieval a second image was obtained to quantify tissue degradation.

FIG. 4 shows detected proteins CD20, CD3e, and CD34 (top) in tonsil tissue immobilized onto solid support including an amine-containing polymer control (e.g., PEI) or a poly PEGMA polymer described herein. Images of the tissue sections were obtained after antigen retrieval. Tonsil samples were further probed with antibody-oligo conjugates specific for CD20, CD3e, and CD34. Aggregates formed at the surface of the glass slide can contribute to elevated background signal. Using a glass slide functionalized with the PEI control, high background signal was observed (bottom). Using a glass slide functionalized the polymer composition described herein, poly PEGMA-NH2, background was low. The number of detected features and average feature signal were quantified using ImageJ.

FIG. 5. A tonsil tissue section detected using the G4X sequencing platform and immuno-oncology panel targeting 300 different mRNA molecules. Each dot represents a detected transcript.

### DETAILED DESCRIPTION

The aspects and embodiments described herein relate to compositions and methods of use of solid supports useful for biological sample (e.g., a cell sample or tissue sample) adherence. As described herein, the methods and compositions of this disclosure have many advantages, including providing a robust surface for tissue adherence for spatial biology applications.

### I. Definitions

All patents, patent applications, articles and publications mentioned herein, both supra and infra, are hereby expressly incorporated herein by reference in their entireties.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art. The following definitions are provided to facilitate understanding of certain terms used frequently herein and are not meant to limit the scope of the present disclosure.

As used herein, the singular terms “a”, “an”, and “the” include the plural reference unless the context clearly indicates otherwise. Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

As used herein, the term “associated” or “associated with” can mean that two or more species are identifiable as being co-located at a point in time. An association can mean that two or more species are or were within a similar container. An association can be an informatics association, where for example digital information regarding two or more species is stored and can be used to determine that one or more of the species were co-located at a point in time. An association can also be a physical association. In some instances two or more associated species are “tethered”, “coated”, “attached”, or “immobilized” to one another or to a common solid or semisolid support. An association may refer to a relationship, or connection, between two entities. Associated may refer to the relationship between a sample and the DNA molecules, RNA molecules, or polynucleotides originating from or derived from that sample. These relationships may be encoded in a label of a detection agent, as described herein. A polynucleotide (e.g., a label) is associated with a particular protein or nucleic acid of interest if the sequence of the polynucleotide is determined a priori and such sequence is associated with a target protein or nucleic acid of interest. A polynucleotide is associated with a sample if it is an endogenous polynucleotide, i.e., it occurs in the sample at the time the sample is obtained, or is derived from an endogenous polynucleotide. For example, the RNAs endogenous to a cell are associated with that cell. cDNAs resulting from reverse transcription of these RNAs, and DNA amplicons resulting from PCR amplification of the cDNAs, contain the sequences of the RNAs and are also associated with the cell. The polynucleotides associated with a sample need not be located or synthesized in the sample, and are considered associated with the sample even after the sample has been destroyed (for example, after a cell has been lysed).

In the description, relative terms such as “before,” “after,” “above,” “below,” “up,” “down,” “top” and “bottom” as well as derivative thereof (e.g., “horizontally,” “downwardly,” “upwardly,” etc.) should be construed to refer to the orientation as then described or as shown in the drawing or figure under discussion. These relative terms are for convenience of description and do not require that the system be constructed or operated in a particular orientation.

As used herein, the term “contacting” is used in accordance with its plain ordinary meaning and refers to the process of allowing at least two distinct species (e.g., chemical compounds, biomolecules, nucleotides, binding reagents, or cells) to become sufficiently proximal to react, interact or physically touch. However, the resulting reaction product can be produced directly from a reaction between the added reagents or from an intermediate from one or more of the added reagents that can be produced in the reaction mixture. The term “contacting” may include allowing two species to react, interact, or physically touch, wherein the two species may be a compound, a protein (e.g., an antibody), substrate, device, or enzyme. In some embodiments contacting includes allowing a tissue slide described herein including a tissue sample to interact with a flow cell.

As used herein, the term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence, only some of the nucleotides of the complement form base pairs with nucleotides of the second nucleic acid sequence. Examples of complementary sequences include coding and non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence. Another example of complementary sequences are a template sequence and an amplicon sequence polymerized by a polymerase along the template sequence.

As described herein, the complementarity of sequences may be partial, in which only some of the nucleic acids match according to base pairing, or complete, where all the nucleic acids match according to base pairing. Thus, two sequences that are complementary to each other, may have a specified percentage of nucleotides that complement one another (e.g., about 60%, preferably 65%, 70%, 75%, 80%, 85%, 90%, 91%, 92%, 93%, 94%, 95%, 96%, 97%, 98%, 99%, or higher complementarity over a specified region). In embodiments, two sequences are complementary when they are completely complementary, having 100% complementarity. In embodiments, one or both sequences in a pair of complementary sequences form portions of longer polynucleotides, which may or may not include additional regions of complementarity.

As used herein, the term “hybridize” or “specifically hybridize” refers to a process where two complementary nucleic acid strands anneal to each other under appropriately stringent conditions. Hybridizations are typically and preferably conducted with oligonucleotides. Non-limiting examples of nucleic acid hybridization techniques are described in, for example, Sambrook, et al., Molecular Cloning: A Laboratory Manual (2nd Edition, 1989). Those skilled in the art understand how to estimate and adjust the stringency of hybridization conditions such that sequences having at least a desired level of complementarity will stably hybridize, while those having lower complementarity will not. Hybridization reactions can be performed under conditions of different “stringency”. For example, a low stringency hybridization reaction is carried out at about 40° C. in 10×SSC. A moderate stringency hybridization may be performed at about 50° C. in 6×SSC. A high stringency hybridization reaction is generally performed at about 60° C. in 1×SSC. Hybridization reactions can also be performed under “physiological conditions” which is well known to one of skill in the art (e.g., a physiological condition is the temperature, ionic strength, pH and concentration of Mg¹⁺ normally found in vivo). The propensity for hybridization between nucleic acids depends on the temperature and ionic strength of their milieu, the length of the nucleic acids and the degree of complementarity. The effect of these parameters on hybridization is described in, for example, Sambrook J., Fritsch E. F., Maniatis T., Molecular cloning: a laboratory manual, Cold Spring Harbor Laboratory Press, New York (1989). As used herein, hybridization of a primer, or of a DNA extension product, respectively, is extendable by creation of a phosphodiester bond with an available nucleotide or nucleotide analogue capable of forming a phosphodiester bond, therewith. For example, hybridization can be performed at a temperature ranging from 15° C. to 95° C. In some embodiments, the hybridization is performed at a temperature of about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., or about 95° C. In other embodiments, the stringency of the hybridization can be further altered by the addition or removal of components of the buffered solution. As used herein, the term “stringent condition” refers to condition(s) under which a polynucleotide probe or primer will hybridize preferentially to its target sequence, and to a lesser extent to, or not at all to, other sequences. A “stringent hybridization” and “stringent hybridization wash conditions” in the context of nucleic acid hybridization are sequence dependent, and are different under different environmental parameters. In some embodiments nucleic acids, or portions thereof, that are configured to specifically hybridize are often about 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, 99% or more or 100% complementary to each other over a contiguous portion of nucleic acid sequence. A specific hybridization discriminates over non-specific hybridization interactions (e.g., two nucleic acids that a not configured to specifically hybridize, e.g., two nucleic acids that are 80% or less, 70% or less, 60% or less or 50% or less complementary) by about 2-fold or more, often about 10-fold or more, and sometimes about 100-fold or more, 1000-fold or more, 10,000-fold or more, 100,000-fold or more, or 1,000,000-fold or more. Two nucleic acid strands that are hybridized to each other can form a duplex which includes a double-stranded portion of nucleic acid. The phrase “stringent hybridization conditions” refers to conditions under which a primer will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

As used herein, “specifically hybridizes” refers to preferential hybridization under hybridization conditions where two nucleic acids, or portions thereof, that are substantially complementary, hybridize to each other and not to other nucleic acids that are not substantially complementary to either of the two nucleic acids. For example, specific hybridization includes the hybridization of a primer or capture nucleic acid to a portion of a target nucleic acid (e.g., a template, or adapter portion of a template) that is substantially complementary to the primer or capture nucleic acid. In some embodiments nucleic acids, or portions thereof, that are configured to specifically hybridize are often about 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, 99% or more or 100% complementary to each other over a contiguous portion of nucleic acid sequence. A specific hybridization discriminates over non-specific hybridization interactions (e.g., two nucleic acids that a not configured to specifically hybridize, e.g., two nucleic acids that are 80% or less, 70% or less, 60% or less or 50% or less complementary) by about 2-fold or more, often about 10-fold or more, and sometimes about 100-fold or more, 1000-fold or more, 10,000-fold or more, 100,000-fold or more, or 1,000,000-fold or more. Two nucleic acid strands that are hybridized to each other can form a duplex which includes a double stranded portion of nucleic acid.

As may be used herein, the terms “nucleic acid,” “nucleic acid molecule,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may include natural nucleic acid sequences and variants thereof, artificial nucleic acid sequences, or a combination of such sequences. As may be used herein, the terms “nucleic acid oligomer” and “oligonucleotide” are used interchangeably and are intended to include, but are not limited to, nucleic acids having a length of 200 nucleotides or less. In some embodiments, an oligonucleotide is a nucleic acid having a length of 2 to 200 nucleotides, 2 to 150 nucleotides, 5 to 150 nucleotides or 5 to 100 nucleotides. The terms “polynucleotide,” “oligonucleotide,” “oligo” or the like refer, in the usual and customary sense, to a linear sequence of nucleotides. Oligonucleotides are typically from about 5, 6, 7, 8, 9, 10, 12, 15, 25, 30, 40, 50 or more nucleotides in length, up to about 100 nucleotides in length. In some embodiments, an oligonucleotide is a primer configured for extension by a polymerase when the primer is annealed completely or partially to a complementary nucleic acid template. A primer is often a single stranded nucleic acid. In certain embodiments, a primer, or portion thereof, is substantially complementary to a portion of an adapter. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. In some embodiments, an oligonucleotide may be immobilized to a solid support.

The term “messenger RNA” or “mRNA” refers to an RNA that is without introns and is capable of being translated into a polypeptide. The term “RNA” refers to any ribonucleic acid, including but not limited to mRNA, tRNA (transfer RNA), rRNA (ribosomal RNA), and/or noncoding RNA (such as lncRNA (long noncoding RNA)). The term “cDNA” refers to a DNA that is complementary or identical to an RNA, in either single stranded or double stranded form.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

As used herein, the term “polynucleotide template” or “template nucleic acid” refers to any polynucleotide molecule that may be bound by a polymerase and utilized as a template for nucleic acid synthesis. As used herein, the term “polynucleotide primer” refers to any polynucleotide molecule that may hybridize to a polynucleotide template, be bound by a polymerase, and be extended in a template-directed process for nucleic acid synthesis, such as in a PCR or sequencing reaction. A primer can be of any length depending on the particular technique it will be used for. For example, PCR primers are generally between 10 and 40 nucleotides in length. The length and complexity of the nucleic acid fixed onto the nucleic acid template may vary. One of skill can adjust these factors to provide optimum hybridization and signal production for a given hybridization procedure. The primer permits the addition of a nucleotide residue thereto, or oligonucleotide or polynucleotide synthesis therefrom, under suitable conditions. In an embodiment the primer is a DNA primer, i.e., a primer consisting of, or largely consisting of, deoxyribonucleotide residues. The primers are designed to have a sequence that is the complement of a region of template/target DNA to which the primer hybridizes. The addition of a nucleotide residue to the 3′ end of a primer by formation of a phosphodiester bond results in a DNA extension product. The addition of a nucleotide residue to the 3′ end of the DNA extension product by formation of a phosphodiester bond results in a further DNA extension product. In another embodiment the primer is an RNA primer. In embodiments, a primer is hybridized to a target polynucleotide.

In general, the term “target polynucleotide” refers to a nucleic acid molecule or polynucleotide in a starting population of nucleic acid molecules having a target sequence whose presence, amount, and/or nucleotide sequence, or changes in one or more of these, are desired to be determined. In general, the term “target sequence” refers to a nucleic acid sequence on a single strand of nucleic acid. The target sequence may be a portion of a gene, a regulatory sequence, genomic DNA, cDNA, RNA including mRNA, miRNA, rRNA, or others. The target sequence may be a target sequence from a sample or a secondary target such as a product of an amplification reaction. A target polynucleotide is not necessarily any single molecule or sequence. For example, a target polynucleotide may be any one of a plurality of target polynucleotides in a reaction, or all polynucleotides in a given reaction, depending on the reaction conditions. For example, in a nucleic acid amplification reaction with random primers, all polynucleotides in a reaction may be amplified. As a further example, a collection of targets may be simultaneously assayed using polynucleotide primers directed to a plurality of targets in a single reaction. As yet another example, all or a subset of polynucleotides in a sample may be modified by the addition of a primer-binding sequence (such as by the ligation of adapters containing the primer binding sequence), rendering each modified polynucleotide a target polynucleotide in a reaction with the corresponding primer polynucleotide(s).

In some embodiments, a nucleic acid includes a label. As used herein, the term “label” or “labels” is used in accordance with their plain and ordinary meanings and refer to molecules that can directly or indirectly produce or result in a detectable signal either by themselves or upon interaction with another molecule. In embodiments, a label is a nucleic acid sequence associated with a detection agent for the detection of biomolecules of interest in tissue sections or cells. Non-limiting examples of detectable labels include fluorescent dyes, biotin, digoxin, haptens, and epitopes. In general, a dye is a molecule, compound, or substance that can provide an optically detectable signal, such as a colorimetric, luminescent, bioluminescent, chemiluminescent, phosphorescent, or fluorescent signal. In embodiments, the label is a dye. In embodiments, the dye is a fluorescent dye. Non-limiting examples of dyes, some of which are commercially available, include CF® dyes (Biotium, Inc.), Alexa Fluor® dyes (Thermo Fisher), DyLight® dyes (Thermo Fisher), Cy® dyes (GE Healthscience), IRDye® dyes (Li-Cor Biosciences, Inc.), and HiLyte™ dyes (Anaspec, Inc.). In embodiments, a particular nucleotide type is associated with a particular label, such that identifying the label identifies the nucleotide with which it is associated. In embodiments, the label is luciferin that reacts with luciferase to produce a detectable signal in response to one or more bases being incorporated into an elongated complementary strand, such as in pyrosequencing. In embodiment, a nucleotide includes a label (such as a dye). In embodiments, the label is not associated with any particular nucleotide, but detection of the label identifies whether one or more nucleotides having a known identity were added during an extension step (such as in the case of pyrosequencing). Examples of detectable agents (i.e., labels) include imaging agents, including fluorescent and luminescent substances, molecules, or compositions, including, but not limited to, a variety of organic or inorganic small molecules commonly referred to as “dyes,” “labels,” or “indicators.” Examples include fluorescein, rhodamine, acridine dyes, Alexa Fluor® dyes, and cyanine dyes. In embodiments, the detectable moiety is a fluorescent molecule (e.g., acridine dye, cyanine, dye, fluorine dye, oxazine dye, phenanthridine dye, or rhodamine dye). In embodiments, the detectable moiety is a fluorescent molecule (e.g., acridine dye, cyanine, dye, fluorine dye, oxazine dye, phenanthridine dye, or rhodamine dye). The term “cyanine” or “cyanine moiety” as described herein refers to a detectable moiety containing two nitrogen groups separated by a polymethine chain. In embodiments, the cyanine moiety has 3 methine structures (i.e., cyanine 3 or Cy3®). In embodiments, the cyanine moiety has 5 methine structures (i.e., cyanine 5 or Cy5®). In embodiments, the cyanine moiety has 7 methine structures (i.e., cyanine 7 or Cy7®).

As used herein, the term “biomolecule” refers to an agent (e.g., a compound, macromolecule, or small molecule), and the like derived from a biological system (e.g., an organism). The biomolecule may contain multiple individual components that collectively construct the biomolecule, for example, in embodiments, the biomolecule is a polynucleotide wherein the polynucleotide is composed of nucleotide monomers. The biomolecule may be or may include DNA, RNA, organelles, carbohydrates, lipids, proteins, or any combination thereof. These components may be extracellular. In some examples, the biomolecule may be referred to as a clump or aggregate of combinations of components. In some instances, the biomolecule may include one or more constituents of a cell but may not include other constituents of the cell. In embodiments, a biomolecule is a molecule produced by a biological system (e.g., an organism). In embodiments, a biomolecule may be referred to as an analyte. Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In embodiments, the analytes within a cell can be localized to subcellular locations, including, for example, organelles, e.g., mitochondria, Golgi apparatus, endoplasmic reticulum, chloroplasts, endocytic vesicles, exocytic vesicles, vacuoles, lysosomes, etc. In embodiments, analyte(s) can be peptides or proteins, including antibodies and/or enzymes. In embodiments, an analyte can be detected indirectly, such as through detection of an intermediate agent, for example, a ligation product or an analyte capture agent (e.g., an oligonucleotide-conjugated antibody), such as those described herein.

As used herein, the term “biological system” refers to a virus, cell, cell derivative, cell nucleus, cell organelle, cell constituent and the like derived from a biological sample. Examples of a cell organelle include, without limitation, a nucleus, endoplasmic reticulum, a ribosome, a Golgi apparatus, an endoplasmic reticulum, a chloroplast, an endocytic vesicle, an exocytic vesicle, a vacuole, and a lysosome. The biological system (e.g., an organism) may contain multiple individual components, such as viruses, cells, cell derivatives, cell nuclei, cell organelles and cell constituents, including combinations of different of these and other components. The biological system may include DNA, RNA, organelles, proteins, or any combination thereof. These components may be extracellular. In some examples, the biological system may be referred to as a clump or aggregate of combinations of components. In some instances, the biological system may include one or more constituents of a cell but may not include other constituents of the cell. An example of such constituents include nucleus or an organelle. A cell may be a live or viable cell. The live cell may be capable of being cultured, for example, being cultured when enclosed in a gel or polymer matrix or cultured when including a gel or polymer matrix. A biological system may include a single cell and/or a single nuclei from a cell.

The term “organelle” as used herein refers to an entity of cell associated with a particular function. In embodiments, an organelle refers to a specialized subunit within a cell that has a specific function, and is usually separately enclosed within its own lipid bilayer. Examples of organelles include the nucleus, mitochondria, endoplasmic reticulum, Golgi apparatus, lysosomes, and chloroplasts (in plant cells). Although most organelles are functional units within cells, some organelles function extend outside of cells, such as cilia, flagellum, archaellum, and the trichocyst. In embodiments, the organelle is a membrane bound organelle. In embodiments, the organelle is a non-membrane bound organelle. Non-membrane bounded organelles, also called biomolecular complexes, are assemblies of macromolecules such as the ribosome, the spliceosome, the proteasome, the nucleosome, and the centriole. Commonly detected organelles includes the nucleus, which is often visualized using dyes such as DAPI, Hoechst, and SYTO Green, mitochondria are with MitoTracker™ dyes and Rhodamine 123, endoplasmic reticulum (ER) utilizing dyes like ER-Tracker® Green/Red or DiOC6, the Golgi apparatus is stained with BODIPY™ FL C5-Ceramide and NBD C6-Ceramide, lysosomes are typically stained using LysoTracker™ dyes and Acridine Orange, and peroxisomes may be stained with Peroxisome-Tracker® Red and Peroxy Green dyes. Although not membrane-bound, ribosomes may be detected using antibodies such as anti-RPL10 or anti-RPS6. Additionally, the cytoskeleton, specifically actin filaments, is frequently stained to study cell shape with Phalloidin conjugates and Alexa Fluor® Phalloidin being widely used. In embodiments, the organelle is a biomolecular complex including a plurality of subunits. In embodiments, the organelle is a macromolecule. In embodiments, the organelle is a eukaryotic organelle. In embodiments, the organelle is the cell membrane, the endoplasmic reticulum, a flagellum, a Golgi apparatus, a mitochondrion, the nucleus, or a vacuole. In embodiments, the organelle is a lysosome. In embodiments, the organelle is the nucleolus.

The terms “particle” and “bead” are used interchangeably and mean a small body made of a rigid or semi-rigid material. The body can have a shape characterized, for example, as a sphere, oval, microsphere, or other recognized particle shape whether having regular or irregular dimensions. The term “particle” does not indicate any particular shape. The shapes and sizes of a collection of particles may be different or about the same (e.g., within a desired range of dimensions, or having a desired average or minimum dimension). A particle may be substantially spherical (e.g., microspheres) or have a non-spherical or irregular shape, such as cubic, cuboid, pyramidal, cylindrical, conical, oblong, or disc-shaped, and the like. In embodiments, the particle has the shape of a sphere, cylinder, spherocylinder, or ellipsoid. In embodiments, a particle is a microsphere used as a calibration tool to calibrate and assess the z-axis used in imaging-based methods for in situ spatial sequencing applications. In embodiments, the methods described herein include focusing on the beads at different depths to calibrate and image in three dimensions.

A “functionalized” solid support, as used herein, may refer to the post hoc conjugation of a moiety to a functional group on the surface of a solid support.

Lengths and sizes of nanoparticles and functionalized particles as described herein may be measured using Transmission Electron Microscopy. For example, transmission electron microscopy measurements of the various particle samples may be drop coated (5 L) onto 200 mesh copper EM grids, air-dried and imaged using a FEI Tecnai 12 TEM equipped with a Gatan Ultrascan 2K CCD camera at an accelerating voltage of 120 kV. The average size distributions of the particles may then be obtained from the TEM images using Image J software that were plotted using software (e.g., Origin Pro 8) to obtain the histogram size distributions of the particles. In embodiment, the length of a nanoparticle refers to the longest dimension of the particle.

As used herein, the term “polymer” refers to macromolecules having one or more structurally unique repeating units. The repeating units are referred to as “monomers,” which are polymerized for the polymer. Typically, a polymer is formed by monomers linked in a chain-like structure. A polymer formed entirely from a single type of monomer is referred to as a “homopolymer.” A polymer formed from two or more unique repeating structural units may be referred to as a “copolymer.” For example, a copolymer formed from three different monomers may also be referred to as a “terpolymer,” which is a subset of copolymers. Thus, unless otherwise specified, the term “copolymer” as used herein encompasses polymers formed from two, three, or more distinct monomer species. A polymer may be linear or branched, and may be random, block, polymer brush, hyperbranched polymer, bottlebrush polymer, dendritic polymer, or polymer micelles. The term “polymer” includes homopolymers, copolymers, tripolymers, tetra polymers and other polymeric molecules made from monomeric subunits. Copolymers include alternating copolymers, periodic copolymers, statistical copolymers, random copolymers, block copolymers, linear copolymers and branched copolymers. The term “polymerizable monomer” is used in accordance with its meaning in the art of polymer chemistry and refers to a compound that may covalently bind chemically to other monomer molecules (such as other polymerizable monomers that are the same or different) to form a polymer.

Polymers can be hydrophilic, hydrophobic or amphiphilic, as known in the art. Thus, “hydrophilic polymers” are substantially miscible with water and include, but are not limited to, polyethylene glycol and the like. “Hydrophobic polymers” are substantially immiscible with water and include, but are not limited to, polyethylene, polypropylene, polybutadiene, polystyrene, polymers disclosed herein, and the like. “Amphiphilic polymers” have both hydrophilic and hydrophobic properties and are typically copolymers having hydrophilic segment(s) and hydrophobic segment(s). Polymers include homopolymers, random copolymers, and block copolymers, as known in the art. The term “homopolymer” refers, in the usual and customary sense, to a polymer having a single monomeric unit. The term “copolymer” refers to a polymer derived from two or more monomeric species. The term “random copolymer” refers to a polymer derived from two or more monomeric species with no preferred ordering of the monomeric species. The term “block copolymer” refers to polymers having two or homopolymer subunits linked by covalent bond. Thus, the term “hydrophobic homopolymer” refers to a homopolymer which is hydrophobic. The term “hydrophobic block copolymer” refers to two or more homopolymer subunits linked by covalent bonds and which is hydrophobic.

As used herein, the term “hydrogel” refers to a three-dimensional polymeric structure that is substantially insoluble in water, but which is capable of absorbing and retaining large quantities of water to form a substantially stable, often soft and pliable, structure. In embodiments, water can penetrate in between polymer chains of a polymer network, subsequently causing swelling and the formation of a hydrogel. In embodiments, hydrogels are super-absorbent (e.g., containing more than about 90% water) and can include natural or synthetic polymers. In some embodiments, the hydrogel polymer includes 60-90% fluid, such as water, and 10-30% polymer. In certain embodiments, the water content of hydrogel is about 70-80%.

Hydrogels may be prepared by cross-linking hydrophilic biopolymers or synthetic polymers. Thus, in some embodiments, the hydrogel may include a crosslinker. As used herein, the term “crosslinker” refers to a molecule that can form a three-dimensional network when reacted with the appropriate base monomers. Examples of the hydrogel polymers, which may include one or more crosslinkers, include but are not limited to, hyaluronans, chitosans, agar, heparin, sulfate, cellulose, alginates (including alginate sulfate), collagen, dextrans (including dextran sulfate), pectin, carrageenan, polylysine, gelatins (including gelatin type A), agarose, (meth)acrylate-oligolactide-PEO-oligolactide-(meth)acrylate, PEO-PPO-PEO copolymers (Pluronics), poly(phosphazene), poly(methacrylates), poly(N-vinylpyrrolidone), PL(G)A-PEO-PL(G)A copolymers, poly(ethylene imine), polyethylene glycol (PEG)-thiol, PEG-acrylate, acrylamide, N,N′-bis(acryloyl)cystamine, PEG, polypropylene oxide (PPO), polyacrylic acid, poly(hydroxyethyl methacrylate) (PHEMA), poly(methyl methacrylate) (PMMA), poly(N-isopropylacrylamide) (PNIPAAm), poly(lactic acid) (PLA), poly(lactic-co-glycolic acid) (PLGA), polycaprolactone (PCL), poly(vinylsulfonic acid) (PVSA), poly(L-aspartic acid), poly(L-glutamic acid), bisacrylamide, diacrylate, diallylamine, triallylamine, divinyl sulfone, diethyleneglycol diallyl ether, ethyleneglycol diacrylate, polymethyleneglycol diacrylate, polyethyleneglycol diacrylate, trimethylopropoane trimethacrylate, ethoxylated trimethylol triacrylate, or ethoxylated pentaerythritol tetracrylate, or combinations thereof. Thus, for example, a combination may include a polymer and a crosslinker, for example polyethylene glycol (PEG)-thiol/PEG-acrylate, acrylamide/N,N′-bis(acryloyl)cystamine (BACy), or PEG/polypropylene oxide (PPO).

As used herein, the term “infrared (IR) reflective coating” refers to a material deposited onto a solid support capable of reflecting some or all infrared light. The effectiveness of an IR reflective coating is noted in its capability to reflect light that falls within the infrared spectrum, specifically light with wavelengths ranging from about 750 nanometers (nm) to about 1,000 micrometers (μm). Examples of IR reflective coating include, but are not limited to, metal oxides and silver. In embodiments, the infrared (IR) reflective coatings may include materials such as gold, aluminum, tantalum oxide, chromium, zinc sulfide, and titanium dioxide. Gold is known for its excellent reflectivity, particularly in the near-infrared range; aluminum is a lightweight metal with a natural oxide layer that enhances its IR reflectivity; chromium, a metal noted for its durable and reflective characteristics, zinc sulfide, a compound frequently used in optical components due to its transparency and reflectivity in the infrared range, and titanium dioxide, a compound widely used for its high refractive index and strong IR reflective properties, are exemplary of the diverse range of materials that can be employed as IR reflective coatings. In embodiments, the infrared (IR) reflective coating includes one or more layers of silicon dioxide (SiO₂) and tantalum pentoxide (Ta₂O₅). The IR reflective coating may reflect a portion of the total radiation.

As used herein, the term “interfacial”, or “interfacial layer”, is used in accordance with its plain ordinary meaning and refers to the boundary between any two bulk phases (gas, liquid, or solid) in contact where the properties differ from the properties of the bulk phases. In embodiments, an interfacial layer includes water. Interfacial water differs from bulk water in a number of properties, for example, interfacial water has a higher heat capacity than bulk water because more energy is necessary to break its hydrogen bonds. The arrangement and structure of the interfacial water layer varies depending on the structure of the hydrophilic and/or hydrophobic surface(s) the water layer is in contact with. Additional properties of interfacial water may be found in, e.g., Mentre P. J. Biol. Phys. and Chem. 2004; 4: 115-123 and Tanaka M. Front. Chem. 2020; 8:165, which are incorporated herein by reference in their entirety.

As used herein, the terms “solid support” and “substrate” and “substrate surface” and “solid surface” refers to discrete solid or semi-solid substrate. In embodiments, a plurality of functional groups (e.g., bioconjugate reactive moieties or polymer) may be attached to the substrate. A solid support may encompass any type of solid, porous, or hollow sphere, ball, cylinder, or other similar configuration composed of plastic, ceramic, metal, or polymeric material (e.g., hydrogel) onto which a nucleic acid may be immobilized (e.g., covalently or non-covalently). A solid support may include a discrete particle that may be spherical (e.g., microspheres) or have a non-spherical or irregular shape, such as cubic, cuboid, pyramidal, cylindrical, conical, oblong, or disc-shaped, and the like. A bead can be non-spherical in shape. A solid support may be used interchangeably with the term “bead.” A solid support may further include a polymer or hydrogel on the surface to which the primers are attached. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefin copolymers, polyimides etc.), nylon, ceramics, resins, Zeonor®, silica or silica-based materials including silicon and modified silicon, carbon, metals, inorganic glasses, optical fiber bundles, photopatternable dry film resists, UV-cured adhesives and polymers. Particularly useful solid supports for some embodiments have at least one surface located on a microplate. Particularly useful solid supports for some embodiments have at least one surface located on a microplate within a flow cell. Solid surfaces can also be varied in their shape depending on the application in a method described herein. For example, a solid surface useful herein can be planar, or contain regions which are concave or convex. In embodiments, the geometry of the concave or convex regions (e.g., wells) of the solid surface conform to the size and shape of a substantially circular particle to maximize the contact between the particle. In embodiments, the wells of an array are randomly located such that nearest neighbor wells have random spacing between each other. Alternatively, in embodiments the spacing between the wells can be ordered, for example, forming a regular pattern. The term solid substrate is encompassing of a substrate (e.g., a microplate or flow cell) having a surface including a polymer coating covalently attached thereto.

Broadly speaking, for nucleic acid sequencing applications and spatial biology, a flow cell may be considered a reaction chamber that contains one or more nucleic acid templates, to which nucleotides and ancillary reagents are iteratively applied and washed away. The flow cell allows for imaging of the sites at which the nucleic acids are bound, and resulting image data is used for the desired analysis.

In embodiments, the solid substrate is a flow cell. The term “flow cell” as used herein refers to a chamber including a solid surface across which one or more fluid reagents can be flowed. Examples of flow cells and related fluidic systems and detection platforms that can be readily used in the methods of the present disclosure are described, for example, in Bentley et al., Nature 456:53-59 (2008). In certain embodiments a substrate includes a surface (e.g., a surface of a flow cell, a surface of a tube, a surface of a chip), for example a metal surface (e.g., steel, gold, silver, aluminum, silicon and copper). In embodiments a substrate (e.g., a substrate surface) is coated and/or includes functional groups and/or inert materials. In certain embodiments a substrate includes a bead, a chip, a capillary, a plate, a membrane, a wafer (e.g., silicon wafers), a comb, or a pin for example. In some embodiments a substrate includes a bead and/or a nanoparticle. A substrate can be made of a suitable material, non-limiting examples of which include a plastic or a suitable polymer (e.g., polycarbonate, poly(vinyl alcohol), poly(divinylbenzene), polystyrene, polyamide, polyester, polyvinylidene difluoride (PVDF), polyethylene, polyurethane, polypropylene, and the like), borosilicate, glass, nylon, Wang resin, Merrifield resin, metal (e.g., iron, a metal alloy, sepharose, agarose, polyacrylamide, dextran, cellulose and the like or combinations thereof. In embodiments a substrate includes a magnetic material (e.g., iron, nickel, cobalt, platinum, aluminum, and the like). In embodiments a substrate includes a magnetic bead (e.g., DYNABEADS®, hematite, AMPure XP). Magnets can be used to purify and/or capture nucleic acids bound to certain substrates (e.g., substrates including a metal or magnetic material). The flow cell is typically a glass slide containing small fluidic channels (e.g., a glass slide 75 mm×25 mm×1 mm having one or more channels), through which sequencing solutions (e.g., polymerases, nucleotides, and buffers) may traverse. Though typically glass, suitable flow cell materials may include polymeric materials, plastics, silicon, quartz (fused silica), Borofloat® glass, silica, silica-based materials, carbon, metals, an optical fiber or optical fiber bundles, sapphire, or plastic materials such as COCs and epoxies. The particular material can be selected based on properties desired for a particular use. For example, materials that are transparent to a desired wavelength of radiation are useful for analytical techniques that will utilize radiation of the desired wavelength. Conversely, it may be desirable to select a material that does not pass radiation of a certain wavelength (e.g., being opaque, absorptive, or reflective). In embodiments, the material of the flow cell is selected due to the ability to conduct thermal energy. In embodiments, a flow cell includes inlet and outlet ports and a flow channel extending there between. In embodiments, the term “flow cell” refers to a vessel having a chamber (e.g., a flow channel or “lane”) where a reaction can be carried out, an inlet for delivering reagent(s) to the chamber, and an outlet for removing reagent(s) from the chamber.

As used herein, the term “channel” refers to a passage in or on a substrate material that directs the flow of a fluid. A channel may run along the surface of a substrate, or may run through the substrate between openings in the substrate. A channel can have a cross section that is partially or fully surrounded by substrate material (e.g., a fluid impermeable substrate material). For example, a partially surrounded cross section can be a groove, trough, furrow or gutter that inhibits lateral flow of a fluid. The transverse cross section of an open channel can be, for example, U-shaped, V-shaped, curved, angular, polygonal, or hyperbolic. A channel can have a fully surrounded cross section such as a tunnel, tube, or pipe. A fully surrounded channel can have a rounded, circular, elliptical, square, rectangular, or polygonal cross section. In particular embodiments, a channel can be located in a flow cell, for example, being embedded within the flow cell. A channel in a flow cell can include one or more windows that are transparent to light in a particular region of the wavelength spectrum. In embodiments, the channel contains one or more polymers of the disclosure. In embodiments, the channel is filled by the one or more polymers, and flow through the channel (e.g., as in a sample fluid) is directed through the polymer in the channel. In embodiments, the tissue is in a channel of a flow cell.

As used herein, the term “gasket” refers to an element that separates the first solid support and the second solid support to define a reaction chamber on the second solid support, wherein the reaction chamber includes a defined gap or channel through which liquid can flow or be contained. In embodiments, a gasket is a spacer element. In embodiments, the thickness (also referred herein as the “depth” or “height” of the channel) may be altered by modulating the height of the gasket or spacer element. In embodiments, the gasket or spacer element includes a peel-off backing designed to form a sealed reaction chamber on the second solid support when adhered to the first solid support. This design ensures the creation of defined channels necessary for fluid flow and biochemical reactions within the assembled flow cell (e.g., flow cell assembly described herein). An example of a gasket or spacer element includes, but is not limited to, those used in the NovaSeq™6000 S4 flow cells, commercialized by Illumina®, which is depicted in Poovathingal et al. (doi: 10.1101/2024.02.22.581576).

Typically, the nucleic acids need to be amplified. In embodiments the term “amplified” refers to a method that includes a polymerase chain reaction (PCR). Conditions conducive to amplification (i.e., amplification conditions) are well known and often include at least a suitable polymerase, a suitable template, a suitable primer or set of primers, suitable nucleotides (e.g., dNTPs), a suitable buffer, and application of suitable annealing, hybridization and/or extension times and temperatures. Amplification conditions may cycle between different temperatures, often involving a large temperature gradient (e.g., 20° C.-40° C.). Additionally, samples embedded in formalin may require additional protocols to render biomolecules available. Heat induced epitope retrieval (HIER) uses heat coupled with buffered solutions to recover antigen reactivity in formalin fixed paraffin embedded tissue samples. Typical HIER methods include increasing the temperature from 25° C. to 95° C.-120° C., if utilizing a water bath or pressure enhanced temperature device (e.g., a pressure cooker). In embodiments, the microplate includes a microplate insert and a planar support attached to the microplate insert. In embodiments, the planar support can include glass (e.g., a glass slide) that has been coated with a substance or otherwise modified to confer conductive properties to the glass. In some embodiments, a glass slide can be coated with a conductive coating. In some embodiments, a conductive coating includes tin oxide (TO) or indium tin oxide (ITO). In some embodiments, a conductive coating includes a transparent conductive oxide (TCO). In some embodiments, a conductive coating includes aluminum doped zinc oxide (AZO). In some embodiments, a conductive coating includes fluorine doped tin oxide (FTO).

As used herein, the term “reaction chamber” refers to a contained space or vessel designed for conducting chemical, biological, or physical reactions. A reaction chamber may include features such as inlets and outlets for introducing and removing substances, sensors for monitoring reaction conditions, and mechanisms for agitation or mixing. In embodiments, the reaction chamber is a part of the flow cell where the cell or tissue is in contact with the fluids (e.g., buffers), polymerases, nucleotides, and reagents used for the methods described herein. In embodiments, the reaction chamber is formed when a first solid support and a second solid support configured to provide a channel are attached together. In embodiments, the reaction chamber is an enclosed (i.e., closed) container containing one or two openings for introducing and removing fluids and reagents.

The term “surface” is intended to mean an external part or external layer of a substrate. The surface can be in contact with another material such as a gas, liquid, gel, polymer, organic polymer, second surface of a similar or different material, metal, or coat. The surface, or regions thereof, can be substantially flat. The substrate and/or the surface can have surface features such as wells, pits, channels, ridges, raised regions, pegs, posts or the like.

As used herein, the term “feature” refers a point or area in a pattern that can be distinguished from other points or areas according to its relative location. An individual feature can include one or more polynucleotides. For example, a feature can include a single target nucleic acid molecule having a particular sequence or a feature can include several nucleic acid molecules having the same sequence (and/or complementary sequence, thereof). Different molecules that are at different features of a pattern can be differentiated from each other according to the locations of the features in the pattern. Non-limiting examples of features include wells in a substrate, particles (e.g., beads) in or on a substrate, polymers in or on a substrate, projections from a substrate, ridges on a substrate, or channels in a substrate. In embodiments, the one or more features include a reaction chamber and its contents. In embodiments, the one or more features includes a target (e.g., a nucleic acid, protein, or biomarker), a cell, or a tissue sample. In embodiments, the feature is a nucleotide (e.g., a fluorescently labeled nucleotide). In embodiments, the feature is a nucleic acid. In embodiments, the feature is a protein. In embodiments, the feature is a biomolecule.

As used herein, the terms “sequencing”, “sequence determination”, and “determining a nucleotide sequence”, are used in accordance with their ordinary meaning in the art, and refer to determination of partial as well as full sequence information of the nucleic acid being sequenced, and particular physical processes for generating such sequence information. That is, the term includes sequence comparisons, fingerprinting, and like levels of information about a target nucleic acid, as well as the express identification and ordering of nucleotides in a target nucleic acid. The term also includes the determination of the identification, ordering, and locations of one, two, or three of the four types of nucleotides within a target nucleic acid. As used herein, the term “sequencing cycle” is used in accordance with its plain and ordinary meaning and refers to incorporating one or more nucleotides (e.g., nucleotide analogues) to the 3′ end of a polynucleotide with a polymerase, and detecting one or more labels that identify the one or more nucleotides incorporated. In embodiments, one nucleotide (e.g., a modified nucleotide) is incorporated per sequencing cycle. The sequencing may be accomplished by, for example, sequencing by synthesis, pyrosequencing, and the like. In embodiments, a sequencing cycle includes extending a complementary polynucleotide by incorporating a first nucleotide using a polymerase, wherein the polynucleotide is hybridized to a template nucleic acid, detecting the first nucleotide, and identifying the first nucleotide. In embodiments, to begin a sequencing cycle, one or more differently labeled nucleotides and a DNA polymerase can be introduced. Following nucleotide addition, signals produced (e.g., via excitation and emission of a detectable label) can be detected to determine the identity of the incorporated nucleotide (based on the labels on the nucleotides). Reagents can then be added to remove the 3′ reversible terminator and to remove labels from each incorporated base. Reagents, enzymes, and other substances can be removed between steps by washing. Cycles may include repeating these steps, and the sequence of each cluster is read over the multiple repetitions.

As used herein, the term “extension” or “elongation” is used in accordance with its plain and ordinary meanings and refer to synthesis by a polymerase of a new polynucleotide strand complementary to a template strand by adding free nucleotides (e.g., dNTPs) from a reaction mixture that are complementary to the template in the 5′-to-3′ direction. Extension includes condensing the 5′-phosphate group of the dNTPs with the 3′-hydroxy group at the end of the nascent (elongating) polynucleotide strand.

As used herein, the term “sequencing read” is used in accordance with its plain and ordinary meaning and refers to an inferred sequence of nucleotide bases (or nucleotide base probabilities) corresponding to all or part of a single polynucleotide fragment. A sequencing read may include 1, 5, 10, 20, 30, 40, 50, 60, 70, 80, 90, 100, 150, 200, 250, or more nucleotide bases. In embodiments, a sequencing read includes reading a barcode sequence and a template nucleotide sequence. In embodiments, a sequencing read includes reading a template nucleotide sequence. In embodiments, a sequencing read includes reading a barcode and not a template nucleotide sequence. Reads of length 20-40 base pairs (bp) are referred to as ultra-short. Typical sequencers produce read lengths in the range of 100-500 bp. Read length is a factor which can affect the results of biological studies. For example, longer read lengths improve the resolution of de novo genome assembly and detection of structural variants. In embodiments, a sequencing read includes reading a barcode and a template nucleotide sequence. In embodiments, a sequencing read includes reading a template nucleotide sequence. In embodiments, a sequencing read includes reading a barcode and not a template nucleotide sequence. In embodiments, a sequencing read includes a computationally derived string corresponding to the detected label. In some embodiments, a sequencing read may include 500, 600, 700, 800, 900, 1,000, 1,100, 1,200, 1,300, 1,400, 1,500, or more nucleotide bases.

Where a range of values is provided, it is understood that each intervening value, to the tenth of the unit of the lower limit unless the context clearly indicates otherwise, between the upper and lower limit of that range, and any other stated or unstated intervening value in, or smaller range of values within, that stated range is encompassed within the invention. The upper and lower limits of any such smaller range (within a more broadly recited range) may independently be included in the smaller ranges, or as particular values themselves, and are also encompassed within the invention, subject to any specifically excluded limit in the stated range. Where the stated range includes one or both of the limits, ranges excluding either or both of those included limits are also included in the invention.

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di-, and multivalent radicals. The alkyl may include a designated number of carbons (e.g., C₁-C₁₀ means one to ten carbons). In embodiments, the alkyl is fully saturated. In embodiments, the alkyl is monounsaturated. In embodiments, the alkyl is polyunsaturated. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkenyl includes one or more double bonds. An alkynyl includes one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. The term “alkynylene” by itself or as part of another substituent, means, unless otherwise stated, a divalent radical derived from an alkyne. In embodiments, the alkylene is fully saturated. In embodiments, the alkylene is monounsaturated. In embodiments, the alkylene is polyunsaturated. An alkenylene includes one or more double bonds. An alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —S—CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in addition to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in addition to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from a heteroalkyne. In embodiments, the heteroalkylene is fully saturated. In embodiments, the heteroalkylene is monounsaturated. In embodiments, the heteroalkylene is polyunsaturated. A heteroalkenylene includes one or more double bonds. A heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. A bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. A bicyclic or multicyclic cycloalkenyl ring system refers to multiple rings fused together wherein at least one of the fused rings is a cycloalkenyl ring and wherein the multiple rings are attached to the parent molecular moiety through any carbon atom contained within a cycloalkenyl ring of the multiple rings.

In embodiments, the term “heterocycloalkyl” means a monocyclic, bicyclic, or a multicyclic heterocycloalkyl ring system. In embodiments, heterocycloalkyl groups are fully saturated. A bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

The symbol [symbol] denotes the point of attachment of a chemical moiety to the remainder of a molecule or chemical formula.

The term “oxo,” as used herein, means an oxygen that is double bonded to a carbon atom.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties: (A) oxo, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and/or (B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C6-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: (i) oxo, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and/or (ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: (a) oxo, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and/or (b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound (e.g., nucleotide analogue) is a chemical species set forth in the Examples section, claims, embodiments, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

Where a moiety is substituted (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene), the moiety is substituted with at least one substituent (e.g., a substituent group, a size-limited substituent group, or lower substituent group) and each substituent is optionally different. Additionally, where multiple substituents are present on a moiety, each substituent may be optionally different.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹⁰ substituents are present, each R¹⁰ substituent may be distinguished as R^{10.1}, R^{10.2}, R^{10.3}, R^{10.4}, etc., wherein each of R^{10.1}, R^{10.2}, R^{10.3}, R^{10.4}, etc. is defined within the scope of the definition of R¹⁰ and optionally differently. Where an R moiety, group, or substituent as disclosed herein is attached through the representation of a single bond and the R moiety, group, or substituent is oxo, a person having ordinary skill in the art will immediately recognize that the oxo is attached through a double bond in accordance with the normal rules of chemical valency.

Descriptions of the compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The compounds of the present invention may exist as salts, such as with pharmaceutically acceptable acids. The present invention includes such salts. Non-limiting examples of such salts include hydrochlorides, hydrobromides, phosphates, sulfates, methanesulfonates, nitrates, maleates, acetates, citrates, fumarates, propionate, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art. The neutral forms of the compounds are preferably regenerated by contacting the salt with a base or acid and isolating the parent compound in the conventional manner. The parent form of the compound may differ from the various salt forms in certain physical properties, such as solubility in polar solvents.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

The term “solution” is used in accordance with its plain ordinary meaning in the arts and refers to a liquid mixture in which the minor component (e.g., a solute or compound) is distributed (e.g., uniformly distributed) within the major component (e.g., a solvent).

The term “organic solvent” as used herein is used in accordance with its ordinary meaning in chemistry and refers to a solvent which includes carbon. Non-limiting examples of organic solvents include acetic acid, acetone, acetonitrile, benzene, 1-butanol, 2-butanol, 2-butanone, t-butyl alcohol, carbon tetrachloride, chlorobenzene, chloroform, cyclohexane, 1,2-dichloroethane, diethylene glycol, diethyl ether, diglyme (diethylene glycol, dimethyl ether), 1,2-dimethoxyethane (glyme, DME), dimethylformamide (DMF), dimethyl sulfoxide (DMSO), 1,4-dioxane, ethanol, ethyl acetate, ethylene glycol, glycerin, heptane, hexamethylphosphoramide (HMPA), hexamethylphosphorous, triamide (HMPT), hexane, methanol, methyl t-butyl ether (MTBE), methylene chloride, N-methyl-2-pyrrolidinone (NMP), nitromethane, pentane, petroleum ether (ligroine), 1-propanol, 2-propanol, pyridine, tetrahydrofuran (THF), toluene, triethyl amine, o-xylene, m-xylene, or p-xylene. In embodiments, the organic solvent is or includes chloroform, dichloromethane, methanol, ethanol, tetrahydrofuran, or dioxane.

As used herein, the term “salt” refers to acid or base salts of the compounds described herein. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts. In embodiments, compounds may be presented with a positive charge, for example

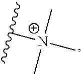 [SMILES: *[N+](C)(C)C]

and it is understood an appropriate counter-ion (e.g., chloride ion, fluoride ion, or acetate ion) may also be present, though not explicitly shown. Likewise, for compounds having a negative charge

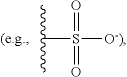

it is understood an appropriate counter-ion (e.g., a proton, sodium ion, potassium ion, or ammonium ion) may also be present, though not explicitly shown. The protonation state of the compound (e.g., a compound described herein) depends on the local environment (i.e., the pH of the environment), therefore, in embodiments, the compound may be described as having a moiety in a protonated state

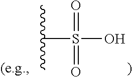 [SMILES: *S(=O)(=O)O]

or an ionic state

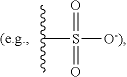

and it is understood these are interchangeable. In embodiments, the counter-ion is represented by the symbol M (e.g., M⁺ or M⁻).

The term “protecting group” is used in accordance with its ordinary meaning in organic chemistry and refers to a moiety covalently bound to a heteroatom, heterocycloalkyl, or heteroaryl to prevent reactivity of the heteroatom, heterocycloalkyl, or heteroaryl during one or more chemical reactions performed prior to removal of the protecting group. An “amine protecting group” refers to a moiety covalently bound to a nitrogen atom to prevent reactivity of the nitrogen atom, during one or more reactions performed prior to removal of the protecting group. Typically a protecting group is bound to a heteroatom (e.g., O or N) during a part of a multipart synthesis or surface deposition protocol wherein it is not desired to have the heteroatom react (e.g., a chemical reduction) with the reagent or surface. Following the desired reaction, the protecting group may be removed (e.g., by modulating the pH or contacting the protecting group with a reducing agent). In embodiments the protecting group is an alcohol protecting group. Non-limiting examples of alcohol protecting groups include acetyl, benzoyl, benzyl, methoxymethyl ether (MOM), tetrahydropyranyl (THP), and silyl ether (e.g., trimethylsilyl (TMS)). In embodiments the protecting group is an amine protecting group. Non-limiting examples of amine protecting groups include carbobenzyloxy (Cbz), tert-butyloxycarbonyl (BOC), 9-Fluorenylmethyloxycarbonyl (FMOC), acetyl, benzoyl, benzyl, carbamate, p-methoxybenzyl ether (PMB), and tosyl (Ts).

The term “leaving group” is used in accordance with its ordinary meaning in chemistry and refers to a moiety (e.g., atom, functional group, or molecule) that separates from the molecule following a chemical reaction (e.g., bond formation, reductive elimination, condensation, or cross-coupling reaction) involving an atom or chemical moiety to which the leaving group is attached, also referred to herein as the “leaving group reactive moiety”, and a complementary reactive moiety (i.e., a chemical moiety that reacts with the leaving group reactive moiety) to form a new bond between the remnants of the leaving groups reactive moiety and the complementary reactive moiety. Thus, the leaving group reactive moiety and the complementary reactive moiety form a complementary reactive group pair. Non limiting examples of leaving groups include hydrogen, hydroxide, halogen (e.g., Br), perfluoroalkylsulfonates (e.g., triflate), tosylates, mesylates, water, alcohols, nitrate, phosphate, thioether, amines, ammonia, fluoride, carboxylate, phenoxides, boronic acid, boronate esters, substituted or unsubstituted piperazinyl, and alkoxides. In embodiments, two molecules are allowed to contact, wherein at least one of the molecules has a leaving group, and upon a reaction and/or bond formation (e.g., acyloin condensation, aldol condensation, Claisen condensation, or Stille reaction) the leaving group(s) separate from the respective molecule. In embodiments, a leaving group is a bioconjugate reactive moiety. In embodiments, the leaving group is designed to facilitate the reaction. In embodiments, the leaving group is a substituent group.

As used herein, the term “barcode” refers to a known nucleic acid sequence that allows some feature with which the barcode is associated to be identified. Typically, a barcode is unique to a particular feature in a pool of barcodes that differ from one another in sequence, and each of which is associated with a different feature. In embodiments, barcodes are about or at least about 1, 2, 3, 4, 5, 6, 7, 8, 9, 10, 15, 20, 25, 30, 40, 50, 75 or more nucleotides in length. In embodiments, barcodes are shorter than 20, 15, 10, 9, 8, 7, 6, or 5 nucleotides in length. In embodiments, barcodes are 10-50 nucleotides in length, such as 15-40 or 20-30 nucleotides in length. In a pool of different barcodes, barcodes may have the same or different lengths. In general, barcodes are of sufficient length and include sequences that are sufficiently different to allow the identification of associated features (e.g., a binding moiety or analyte) based on barcodes with which they are associated. In embodiments, a barcode can be identified accurately after the mutation, insertion, or deletion of one or more nucleotides in the barcode sequence, such as the mutation, insertion, or deletion of 1, 2, 3, 4, 5, or more nucleotides. In embodiments, a detection agent described herein has a barcode, wherein the barcode is an oligonucleotide label. In embodiments, the barcode is a sample barcode. In embodiments, a plurality of nucleotides (e.g., all nucleotides from a particular sample source, or sub-sample thereof) are joined to a first sample barcode, while a different plurality of nucleotides (e.g., all nucleotides from a different sample source, or different subsample) are joined to a second sample barcode, thereby associating each plurality of polynucleotides with a different sample barcode indicative of sample source. In embodiments, each sample barcode in a plurality of sample barcodes differs from every other sample barcode in the plurality by at least three nucleotide positions, such as at least 3, 4, 5, 6, 7, 8, 9, 10, or more nucleotide positions. In some embodiments, substantially degenerate sample barcodes may be known as random. In some embodiments, a sample barcode may include a nucleic acid sequence from within a pool of known sequences. In some embodiments, the sample barcodes may be pre-defined. In embodiments, the sample barcode includes about 1 to about 10 nucleotides. In embodiments, the sample barcode includes about 3, 4, 5, 6, 7, 8, 9, or about 10 nucleotides. In embodiments, the sample barcode includes about 3 nucleotides. In embodiments, the sample barcode includes about 5 nucleotides. In embodiments, the sample barcode includes about 7 nucleotides. In embodiments, the sample barcode includes about 10 nucleotides. In embodiments, the sample barcode includes about 6 to about 10 nucleotides.

As used herein, the term “detection agent” refers to an agent with a label that is capable of specifically binding to a biomolecule of interest to facilitate the detection of the biomolecule of interest. Binding the detection agent to the biomolecule of interest facilitates detecting the label and thus, detection of the biomolecule of interest. An example of a detection agent with a label (e.g., a detectable label) includes fluorescently labeled antibodies used for flow cytometry applications. An additional example of a detection agent with a label is a padlock probe capable of hybridizing to a nucleic acid of interest, where the padlock probe harbors an oligonucleotide label that is sequenced to facilitate the detection of the nucleic acid of interest. In embodiments, the detection agent is a biomolecule-specific binding agent, wherein the biomolecule-specific binding agent specifically binds a target biomolecule.

The terms “detect” and “detecting” as used herein refer to the act of viewing (e.g., imaging, indicating the presence of, quantifying, or measuring (e.g., spectroscopic measurement), an agent based on an identifiable characteristic of the agent, for example, the light emitted from the present compounds. For example, the compound described herein can be bound to an agent, and, upon being exposed to an absorption light, will emit an emission light. The presence of an emission light can indicate the presence of the agent. Likewise, the quantification of the emitted light intensity can be used to measure the concentration of the agent.

In embodiments, the biomolecule-specific binding agent is a protein-specific binding agent. In embodiments, the biomolecule-specific binding agent is an oligonucleotide-specific binding agent. In embodiments, the biomolecule-specific binding agent is capable of binding to a cluster of differentiation (CD) marker, integrin, selectin, cadherin, cytokine receptor, chemokine receptor, Toll-like receptor (TLR), ion channel, transmembrane protein, lipoprotein, glycoprotein, cell surface protein, transport protein, intracellular organelle, or transcription factor. In embodiments, the intracellular organelle includes actin, carbohydrate, centrosomes and centrioles, chloroplasts (in plant cells and some protists), cytoskeleton, endoplasmic reticulum, endosome, Golgi apparatus, intermediate filaments, lysosome, microfilaments, microtubules, mitochondria, nuclear envelope, nuclear pores, nucleoid, nucleolus, nucleus, peroxisome, phosphatidylserine, plasma membrane, ribosomes, rough endoplasmic reticulum, smooth endoplasmic reticulum, transferrin receptor, transport vesicles, and/or vacuoles. In embodiments, the biomolecule specific binding agent is capable of binding to a biomolecule in the mitogen-activated protein kinase (MAPK) pathway, PI3K/AKT/mTOR pathway, Wnt/β-catenin pathway, intrinsic (mitochondrial) pathway, extrinsic (death receptor) pathway, caspase cascade, Notch signaling pathway, hedgehog signaling pathway, TGF-β (transforming growth factor Beta) pathway, JAK/STAT pathway, G-protein coupled receptor (GPCR) pathway, calcium signaling pathway, glycolysis, citric acid cycle (Krebs Cycle), oxidative phosphorylation, lipid metabolism pathway, amino acid metabolism, Toll-like receptor (TLR) pathway, NF-κB signaling pathway, complement pathway, nucleotide excision repair (NER), base excision repair (BER), mismatch repair (MMR), cyclin-dependent kinase (CDK) pathway, Rb (retinoblastoma) pathway, p53 pathway, unfolded protein response (UPR), heat shock response pathway, oxidative stress pathway, BMP (bone morphogenetic protein) pathway, FGF (fibroblast growth factor) pathway, Sonic Hedgehog pathway, neurotrophin signaling pathway, synaptic transmission pathway, axon guidance pathways, insulin signaling pathway, thyroid hormone pathway, steroid hormone pathway, VEGF (vascular endothelial growth factor) pathway, DNA methylation pathway, histone modification pathway, or angiogenesis. In embodiments, the biomolecule specific binding agent is capable of binding to a biomolecule on the surface of or in a B cell, Mature B Cell, Follicular B cell, Marginal Zone B cell, Short lived plasma cell, Memory B cell, Long lived plasma cell, B1 cell, Breg, Germinal Center B cell, Macrophage, Monocyte, M1 macrophage, M2 macrophage, Dendritic Cell, Plasmacytoid dendritic cell, Monocyte-derived dendritic cell, T cell, T Follicular Helper, Th1, Th2, Th9, Th17, Th22, Treg, platelet (activated), platelet (rested), natural killer cell, neutrophil, basophil, eosinophil, mast cell, astrocyte, neuron, glial cell, lymphocyte, myeloid cell, granulocytes, neural cells, stem cells, endothelial cells, epithelial cells, mesenchymal stem cell, hematopoietic stem cell, embryonic stem, stromal cell, erythrocyte, fibroblast, or apoptotic cell.

As used herein, the term “protein-specific binding agent” refers to a molecule or agent that recognizes and binds to a protein or specific part of a protein with high affinity and specificity. Examples of a protein-specific protein binding agent include, but are not limited to, antibodies, aptamers, enzyme inhibitors, ligands for receptors, affinity tags, peptide-based protein binding agents, chelating agents for metalloproteins, and RNA interference agents. In embodiments, a protein-specific binding agent includes a protein-specific antibody conjugated to an oligonucleotide (referred herein as “protein-specific antibody-oligo (Ab-O) conjugates”), wherein the oligonucleotide in the protein-specific antibody-oligo is an oligonucleotide label as described herein.

As used herein, the term “oligonucleotide-specific binding agent” refers to a molecule (e.g., an oligonucleotide) capable of hybridizing to specific sequences of nucleotides. Examples include but are not limited to antisense oligonucleotides, aptamers, and small interfering RNA molecules.

As used herein, the term “oligonucleotide label” or “label” refers to a known nucleic acid sequence that is associated with a detection agent and that allows the target of the detection agent with which the oligonucleotide label or label is associated to be identified. In embodiments, the oligonucleotide label is a detectable label. The sequence oligonucleotide label or label is determined a priori and the identity of a biomolecule of interest (e.g., a protein or nucleic acid) is determined following the binding of the detection agent to the biomolecule of interest, detecting the sequence of the label, and associating the detection of the sequence of the label with the biomolecule of interest. In embodiments, detecting the sequence of the oligonucleotide label or label includes sequencing.

The terms “bind” and “bound” as used herein are used in accordance with their plain and ordinary meanings and refer to an association between atoms or molecules. The association can be direct or indirect. For example, bound atoms or molecules may be directly bound to one another, e.g., by a covalent bond or non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). As a further example, two molecules may be bound indirectly to one another by way of direct binding to one or more intermediate molecules (e.g., as in a substrate, bound to a first antibody, bound to an analyte, bound to a second antibody), thereby forming a complex. As used herein, the term “attached” refers to the state of two things being joined, fastened, adhered, connected or bound to each other. For example, a sample such as a cell or tissue, can be attached to a material, such as a hydrogel, polymer, or solid support, by a covalent or non-covalent bond. In embodiments, attachment is a covalent attachment.

Where a range of values is provided, it is understood that each intervening value, to the tenth of the unit of the lower limit unless the context clearly indicates otherwise, between the upper and lower limit of that range, and any other stated or unstated intervening value in, or smaller range of values within, that stated range is encompassed by such disclosure herein. The upper and lower limits of any such smaller range (within a more broadly recited range) may independently be included in the smaller ranges, or as particular values themselves, and are also encompassed by such disclosure herein, subject to any specifically excluded limit in the stated range. Where the stated range includes one or both of the limits, ranges excluding either or both of those included limits are also included by such disclosure herein.

Provided herein are methods, systems, and compositions for analyzing a sample (e.g., sequencing nucleic acids within a sample) in situ. The term “in situ” is used in accordance with its ordinary meaning in the art and refers to a sample surrounded by at least a portion of its native environment, such as may preserve the relative position of two or more elements. For example, an extracted human cell obtained is considered in situ when the cell is retained in its local microenvironment so as to avoid extracting the target (e.g., nucleic acid molecules or proteins) away from their native environment. An in situ sample (e.g., a cell) can be obtained from a suitable subject. An in situ cell sample may refer to a cell and its surrounding milieu, or a tissue. A sample can be isolated or obtained directly from a subject or part thereof. In embodiments, the methods described herein (e.g., sequencing a plurality of target nucleic acids of a cell in situ) are applied to an isolated cell (i.e., a cell not surrounded by least a portion of its native environment). For the avoidance of any doubt, when the method is performed within a cell (e.g., an isolated cell) the method may be considered in situ. In some embodiments, a sample is obtained indirectly from an individual or medical professional. A sample can be any specimen that is isolated or obtained from a subject or part thereof. A sample can be any specimen that is isolated or obtained from multiple subjects. Non-limiting examples of specimens include fluid or tissue from a subject, including, without limitation, blood or a blood product (e.g., serum, plasma, platelets, buffy coats, or the like), umbilical cord blood, chorionic villi, amniotic fluid, cerebrospinal fluid, spinal fluid, lavage fluid (e.g., lung, gastric, peritoneal, ductal, ear, arthroscopic), a biopsy sample, celocentesis sample, cells (blood cells, lymphocytes, placental cells, stem cells, bone marrow derived cells, embryo or fetal cells) or parts thereof (e.g., mitochondrial, nucleus, extracts, or the like), urine, feces, sputum, saliva, nasal mucous, prostate fluid, lavage, semen, lymphatic fluid, bile, tears, sweat, breast milk, breast fluid, the like or combinations thereof. Non-limiting examples of tissues include organ tissues (e.g., liver, kidney, lung, thymus, adrenals, skin, bladder, reproductive organs, intestine, colon, spleen, brain, the like or parts thereof), epithelial tissue, hair, hair follicles, ducts, canals, bone, eye, nose, mouth, throat, ear, nails, the like, parts thereof or combinations thereof. A sample may include cells or tissues that are normal, healthy, diseased (e.g., infected), and/or cancerous (e.g., cancer cells). A sample obtained from a subject may include cells or cellular material (e.g., nucleic acids) of multiple organisms (e.g., virus nucleic acid, fetal nucleic acid, bacterial nucleic acid, parasite nucleic acid). A sample may include a cell and RNA transcripts. A sample may include a cell and DNA. A sample may include a cell and target proteins. A sample may include DNA, RNA, organelles, carbohydrates, lipids, proteins, or any combination thereof. These components may be extracellular. A sample may include a “target” of the method described herein or any embodiments of the method described herein. A sample may include any compound that may be desired to be detected, for example a peptide or protein, or nucleic acid molecule or a small molecule, including organic and inorganic molecules. A sample may include proteinaceous molecules such as peptides, polypeptides, proteins or prions or any molecule which includes a protein or polypeptide component, etc., or fragments thereof. A sample may include a single molecule or a complex that contains two or more molecular subunits, which may or may not be covalently bound to one another, and which may be the same or different. Thus, in addition to cells or microorganisms, a sample may also include a protein complex. Such a complex may thus be a homo- or hetero-multimer. Aggregates of molecules e.g., proteins may also be target analytes, for example aggregates of the same protein or different proteins. A sample may include a complex between proteins or peptides and nucleic acid molecules such as DNA or RNA. Of particular interest may be the interactions between proteins and nucleic acids, e.g., regulatory factors, such as transcription factors, and interactions between DNA or RNA molecules. A sample can include nucleic acids obtained from one or more subjects. In some embodiments a sample includes nucleic acid obtained from a single subject. A subject can be any living or non-living organism, including but not limited to a human, non-human animal, plant, bacterium, fungus, virus, or protist. A subject may be any age (e.g., an embryo, a fetus, infant, child, adult). A subject can be of any sex (e.g., male, female, or combination thereof). A subject may be pregnant. In some embodiments, a subject is a mammal. In some embodiments, a subject is a plant. In some embodiments, a subject is a human subject. A subject can be a patient (e.g., a human patient). In some embodiments a subject is suspected of having a genetic variation or a disease or condition associated with a genetic variation. A “tissue section” as used herein refers to a portion of a biological tissue derived from a biological sample, typically from an organism (e.g., a human or animal subject or patient).

As used herein, the term “fresh,” generally in the context of a fresh tissue means that the tissue has recently been obtained from an organism, generally before any subsequent fixation steps, for example, flash freezing or chemical fixation. In embodiments, a fresh tissue is obtained from an organism about 1 second up to about 20 minutes before any fixation steps are performed. In embodiments, a fresh tissue is obtained from an organism about 1 second up to about 60 seconds before any fixation steps are performed. In embodiments, a fresh tissue is obtained from an organism about 30 seconds up to about 60 seconds before any fixation steps are performed. In embodiments, a fresh tissue is obtained from an organism about 1 minutes up to about 20 minutes before any fixation steps are performed. In embodiments, a fresh tissue is obtained from an organism about 1 minutes up to about 10 minutes before any fixation steps are performed. In embodiments, a fresh tissue is obtained from an organism about 1 minutes up to about 5 minutes before any fixation steps are performed. In embodiments, a fresh tissue is obtained from an organism about 30 seconds, about 1 minute, about 2 minutes, about 3 minutes, about 4 minutes, about 5 minutes, about 10 minutes, about 15 minutes, or about 20 minutes before any fixation steps are performed.

As used herein, the term “fix,” refers to formation of covalent bonds, such as crosslinks, between biomolecules or within molecules. The process of fixing tissue samples or biological samples (e.g., cells and nuclei) for example, is called “fixation.” The agent that causes fixation is generally referred to as a “fixative” or “fixing agent.” “Fixed biological samples” (e.g., fixed cells or nuclei) or “fixed tissues” refers to biological samples (e.g., cells or nuclei) or tissues that have been in contact with a fixative under conditions sufficient to allow or result in formation of intra- and inter-molecular crosslinks between biomolecules in the biological sample. Fixation may be reversed and the process of reversing fixation may be referred to as “un-fixing” or “decrosslinking.” Unfixing or decrosslinking refers to breaking or reversing the formation of covalent bonds in biomolecules formed by fixatives. In some examples, the tissue fixed is fresh tissue. In some examples, the tissue fixed may be frozen tissue. In some examples, the tissue fixed may not be dissociated. In some examples, the tissue fixed may be dissociated or partially dissociated (e.g., chopped, cut). In some examples, tissue that has been rapidly frozen and, perhaps, cut or chopped into pieces (e.g., small enough to fit into a tube or container used for fixation) may be used. In some examples, tissue may be dissociated or partially dissociated (e.g., cut, chopped) before or during fixation. In some examples, tissue that is fixed may not be dissociated. The frozen biological tissue can be fixed using a fixing agent, which is suitably an organic fixing agent. Suitable organic fixing agents include, without limitation, alcohols, ketones, aldehydes (e.g., glutaraldehyde), cross-linking agents, disuccinimidyl suberate (DSS), dimethylsuberimidate (DMS), formalin, dimethyladipimidate (DMA), dithio-bis(-succinimidyl propionate) (DSP), disuccinimidyl tartrate (DST), ethylene glycol bis (succinimidyl succinate) (EGS), bis(sulfosuccinimidyl)suberate (BS3) and combinations thereof. A particularly suitable fixing agent is a formaldehyde-based fixing agent such as formalin, which is a mixture of formaldehyde and water. The formalin may include about 1% to about 15% by weight formaldehyde and about 85% to about 99% by weight water, suitable about 2% to about 8% by weight formaldehyde and about 92% to about 98% by weight water, or about 4% by weight formaldehyde and about 96% by weight water. In some examples, tissues may be fixed in 4% paraformaldehyde. Other suitable fixing agents will be appreciated by those of ordinary skill in the art (e.g., International PCT App. No. PCT/US2020/066705, which is incorporated herein by reference in its entirety).

As used herein, the term “permeable” refers to a property of a substance that allows certain materials to pass through the substance. “Permeable” may be used to describe a biological sample, such as a cell or nucleus, in which analytes in the biological sample can leave the biological sample. “Permeabilize” is an action taken to cause, for example, a biological sample (e.g., a cell) to release its analytes. In some examples, permeabilization of a biological sample is accomplished by affecting the integrity (e.g., compromising) of a biological sample membrane (e.g., a cellular or nuclear membrane) such as by application of a protease or other enzyme capable of disturbing a membrane allowing analytes to diffuse out of the biological sample. In some embodiments, permeabilizing a biological sample does not release the biomolecules (e.g., proteins and/or nucleic acids) contained within the sample.

As used herein, the term “single biological sample”, such as a single cell or a single nucleus generally refers to a biological sample that is not present in an aggregated form or clump. Single biological samples, such as cells and/or nuclei may be the result of dissociating a tissue sample.

As used herein, the term “tissue freezing” is used in accordance with its plain and ordinary meaning and refers to different methods for freezing tissues. In some examples, the methods used may be rapid methods (e.g., “flash freezing” or “snap freezing”). In some examples, tissues may be lowered to temperatures below about −70° C. using these methods. In some examples, rapid freezing may use ultracold media. In some examples, an ultracold medium may be liquid nitrogen. In some examples, this type of freezing may preserve tissue integrity, in part by preventing the formation of ice crystals that would affect the tissue morphology. In some examples, an ultracold medium may be dry ice.

As used herein, a “single cell” refers to one cell. Single cells useful in the methods described herein can be obtained from a tissue of interest, or from a biopsy, blood sample, or cell culture. Additionally, cells from specific organs, tissues, tumors, neoplasms, or the like can be obtained and used in the methods described herein. In general, cells from any population can be used in the methods, such as a population of prokaryotic or eukaryotic organisms, including bacteria or yeast.

As used herein, the term “tissue” is used in accordance with its plain and ordinary meaning and refers to an organization of cells in a structure, where the structure generally functions as a unit in an organism (e.g., mammals) and may carry out specific functions. In some examples, cells in a tissue are configured in a mass and may not be free from one another. This disclosure describes methods of obtaining single biological samples (e.g., cells or nuclei) from tissues that can be used in various single biological samples (e.g., single-cell/nucleus) workflows. In some examples, blood cells (e.g., lymphocytes) can be considered a tissue. However, blood cells, like lymphocytes, generally are free from one another in the blood. The methods disclosed herein can be used to process those cells to obtain cells and/or nuclei, although dissociation steps may not be necessary when using those types of tissues. Generally, any type of tissue can be used in the methods described herein. Examples of tissues that may be used in the disclosed methods include, but are not limited to connective, epithelial, muscle and nervous tissue. In some examples, the tissues are from mammals. Tissues that contain any type of cells may be used. For example, tissues from abdomen, bladder, brain, esophagus, heart, intestine, kidney, liver, lung, lymph node, olfactory bulb, ovary, pancreas, skin, spleen, stomach, testicle, and the like. The tissue may be normal or tumor tissue (e.g., malignant). This example is not meant to be limiting. Although the conditions used in the disclosed may not be identical for different types of tissue, the methods may be applied to any tissue. The tissues used in the disclosed methods may be in various states. In some examples, the tissues used in the disclosed methods may be fresh, frozen, or fixed.

The term “cellular component” is used in accordance with its ordinary meaning in the art and refers to any organelle, nucleic acid, protein, or analyte that is found in a prokaryotic, eukaryotic, archaeal, or other organismic cell type. Examples of cellular components (e.g., a component of a cell) include RNA transcripts, proteins, membranes, lipids, and other analytes. In embodiments, a cellular component is a biomolecule.

A “gene” refers to a polynucleotide that is capable of conferring biological function after being transcribed and/or translated.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay, etc.) from one location to another. For example, kits include one or more enclosures (e.g., boxes) containing the relevant reaction reagents and/or supporting materials. As used herein, the term “fragmented kit” refers to a delivery system including two or more separate containers that each contain a subportion of the total kit components. The containers may be delivered to the intended recipient together or separately. For example, a first container may contain an enzyme for use in an assay, while a second container contains oligonucleotides. In contrast, a “combined kit” refers to a delivery system containing all of the components of a reaction assay in a single container (e.g., in a single box housing each of the desired components). The term “kit” includes both fragmented and combined kits.

As used herein the term “determine” can be used to refer to the act of ascertaining, establishing or estimating. A determination can be probabilistic. For example, a determination can have an apparent likelihood of at least 50%, 75%, 90%, 95%, 98%, 99%, 99.9% or higher. In some cases, a determination can have an apparent likelihood of 100%. An exemplary determination is a maximum likelihood analysis or report. As used herein, the term “identify,” when used in reference to a thing, can be used to refer to recognition of the thing, distinction of the thing from at least one other thing or categorization of the thing with at least one other thing. The recognition, distinction or categorization can be probabilistic. For example, a thing can be identified with an apparent likelihood of at least 50%, 75%, 90%, 95%, 98%, 99%, 99.9% or higher. A thing can be identified based on a result of a maximum likelihood analysis. In some cases, a thing can be identified with an apparent likelihood of 100%.

The terms “bioconjugate group,” “bioconjugate reactive moiety,” and “bioconjugate reactive group” refer to a chemical moiety which participates in a reaction to form a bioconjugate linker (e.g., covalent linker).

As used herein, the term “bioconjugate reactive moiety” and “bioconjugate reactive group” refers to a moiety or group capable of forming a bioconjugate (e.g., covalent linker) as a result of the association between atoms or molecules of bioconjugate reactive groups. The association can be direct or indirect. For example, a conjugate between a first bioconjugate reactive group (e.g., —NH2, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3^{rd} Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine).

Useful bioconjugate reactive groups used for bioconjugate chemistries herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc.; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized; (i) alkenes, which can undergo, for example, cycloadditions, acylation, Michael addition, etc.; (j) epoxides, which can react with, for example, amines and hydroxyl compounds; (k) phosphoramidites and other standard functional groups useful in nucleic acid synthesis; (l) metal silicon oxide bonding; (m) metal bonding to reactive phosphorus groups (e.g., phosphines) to form, for example, phosphate diester bonds.; (n) azides coupled to alkynes using copper catalyzed cycloaddition click chemistry; (o) biotin conjugate can react with avidin or streptavidin to form a avidin-biotin complex or streptavidin-biotin complex.

The term “covalent linker” is used in accordance with its ordinary meaning and refers to a divalent moiety which connects at least two moieties to form a molecule.

The term “non-covalent linker” is used in accordance with its ordinary meaning and refers to a divalent moiety which includes at least two molecules that are not covalently linked to each other but are capable of interacting with each other via a non-covalent bond (e.g., electrostatic interactions (e.g., ionic bond, hydrogen bond, halogen bond) or van der Waals interactions (e.g., dipole-dipole, dipole-induced dipole, London dispersion). In embodiments, the non-covalent linker is the result of two molecules that are not covalently linked to each other that interact with each other via a non-covalent bond.

An “antibody” (Ab) is a protein that binds specifically to a particular substance, known as an “antigen” (Ag). An “antibody” or “antigen-binding fragment” is an immunoglobulin that binds a specific “epitope.” The term encompasses polyclonal, monoclonal, and chimeric antibodies. In nature, antibodies are generally produced by lymphocytes in response to immune challenge, such as by infection or immunization. An “antigen” (Ag) is any substance that reacts specifically with antibodies or T lymphocytes (T cells). An antibody may include the entire antibody as well as any antibody fragments capable of binding the antigen or antigenic fragment of interest. Examples include complete antibody molecules, antibody fragments, such as Fab, F(ab′)₂, CDRs, VL, VH, and any other portion of an antibody which is capable of specifically binding to an antigen. Antibodies used herein are immunospecific for, and therefore specifically and selectively bind to, for example, proteins either detected (e.g., biological targets of interest) or used for detection (e.g., probes containing oligonucleotide barcodes) in the methods and devices as described herein.

As used herein, the term “control” or “control experiment” is used in accordance with its plain and ordinary meaning and refers to an experiment in which the subjects, cells, tissues, or reagents of the experiment are treated as in a parallel experiment except for omission of a procedure, reagent, or variable of the experiment. In some instances, the control is used as a standard of comparison in evaluating experimental effects. In embodiments, a control cell is the same cell type as the cell being examined, wherein the control cell does not include the variable or is subjected to conditions being examined.

Typically, the concentration and molecular weight of the hydrogel subunit(s) will depend on the selected polymer and the desired characteristics, e.g., pore size, swelling properties, conductivity, elasticity/stiffness (Young's modulus), biodegradability index, etc., of the hydrogel network into which they will be polymerized. For example, it may be desirable for the hydrogel to include pores of sufficient size to allow the passage of macromolecules, e.g., proteins, nucleic acids, or small molecules as described in greater detail below, into the specimen. The ordinarily skilled artisan will be aware that pore size generally decreases with increasing concentration of hydrogel subunits and generally increases with an increasing ratio of hydrogel subunits to crosslinker, and will prepare a hydrogel composition that includes a concentration of hydrogel subunits that allows the passage of such macromolecules. As another example, it may be desirable for the hydrogel to have a particular stiffness, e.g., to provide stability in handling the embedded specimen, e.g., a Young's Modulus (also referred to herein as a compression modulus) of about 2-70 kN/m², for example, about 2 kN/m², about 4 kN/m², about 7 kN/m², about 10 kN/m², about 15 kN/m², about 20 kN/m², about 40 kN/m², but typically not more than about 70 kN/m². The ordinarily skilled artisan will be aware that the elasticity of a hydrogel network may be influenced by a variety of factors, including the branching of the polymer, the concentration of hydrogel subunits, and the degree of cross-linking, and will prepare a hydrogel composition that includes a concentration of hydrogel subunits to provide such desired elasticity. Thus, for example, the hydrogel composition may include an acrylamide monomer at a concentration of from about 1% w/v to about 20% w/v, e.g., about 2% to about 15%, about 3% to about 10%, about 4% to about 8%, and a concentration of bis-acrylamide crosslinker in the range of about 0.01% to about 0.075%, e.g., 0.01%, 0.02%, 0.025%, 0.03%, 0.04%, 0.05%, 0.06%, or 0.075%; or, for example, the hydrogel composition may include PEG prepolymers having a molecular weight ranging from at least about 2.5K to about 50K, e.g., 2.5K or more, 3.5K or more, 5K or more, 7.5K or more, 10K or more, 15K or more, 20K or more, but typically not more than about 50K, at a concentration in a range from about 1% w/w to about 50% w/w, e.g., 1% or more, 5% or more, 7.5% or more, 10% or more, 15% or more, 20% or more, 30% or more, 40% or more, and usually not more than about 50%. Concentrations of hydrogel subunits that provide desired hydrogel characteristics may be readily determined by methods in the art or as described in the working examples below.

The term “image” is used according to its ordinary meaning and refers to a representation of all or part of an object. The representation may be an optically detected reproduction. For example, an image can be obtained from fluorescent, luminescent, scatter, or absorption signals. The part of the object that is present in an image can be the surface or other xy plane of the object. Typically, an image is a 2 dimensional representation of a 3 dimensional object. An image may include signals at differing intensities (i.e., signal levels). An image can be provided in a computer readable format or medium. An image is derived from the collection of focus points of light rays coming from an object (e.g., the sample), which may be detected by any image sensor.

As used herein, the term “signal” is intended to include, for example, fluorescent, luminescent, scatter, or absorption impulse or electromagnetic wave transmitted or received. Signals can be detected in the ultraviolet (UV) range (about 200 to 390 nm), visible (VIS) range (about 391 to 770 nm), infrared (IR) range (about 0.771 to 25 microns), or other range of the electromagnetic spectrum. The term “signal level” refers to an amount or quantity of detected energy or coded information. For example, a signal may be quantified by its intensity, wavelength, energy, frequency, power, luminance, or a combination thereof. Other signals can be quantified according to characteristics such as voltage, current, electric field strength, magnetic field strength, frequency, power, temperature, etc. Absence of signal is understood to be a signal level of zero or a signal level that is not meaningfully distinguished from noise.

The term “xy coordinates” refers to information that specifies location, size, shape, and/or orientation in an xy plane. The information can be, for example, numerical coordinates in a Cartesian system. The coordinates can be provided relative to one or both of the x and y axes or can be provided relative to another location in the xy plane (e.g., a fiducial). The term “xy plane” refers to a 2 dimensional area defined by straight line axes x and y. When used in reference to a detecting apparatus and an object observed by the detector, the xy plane may be specified as being orthogonal to the direction of observation between the detector and object being detected.

As used herein, the term “feature” refers a site (i.e., a physical location) in a tissue or cell on a solid support for one or more molecule(s). A feature can contain only a single molecule or it can contain a population of several molecules of the same species (i.e., a cluster). Features of an array are typically discrete. The discrete features can be contiguous, or they can have spaces between each other. An “optically resolved volume” refers to a three-dimensional region in a cell or tissue with a feature or plurality of features capable of being distinguished from other features.

The term “adhesion strength” or “attachment strength” as used herein refers to the interfacial force bonding two materials together. The adhesion strength may refer to the minimal amount of force necessary to detach and/or remove the two materials. Means for quantifying adhesion strength are known in the art, for example with a pull-off adhesion test. A pull-off adhesion test measures the resistance of a substance (e.g., a tissue sample) from a substrate (e.g., a carrier substrate) when a perpendicular tensile force is applied to the substance. As outlined in the American Society for Testing and Materials (ASTM) D4541 (and similarly in BS EN ISO 4624), the test may include attaching a test dolly to the substance (e.g., the tissue sample) and then pulling the dolly by exerting a force perpendicular to the surface in an effort to remove the dolly with the substance from the substrate. An alternative testing approach is outlined in ASTM D6677 which utilizes a utility knife to peel the substance away from the substrate and ASTM D3359 which uses a pressure sensitive tape. The peel strength tests employed for examining the strength of Band-Aid® bonds are provided in ASTM D903, ASTM D1876, and ASTM F2258, each of which are incorporated herein by reference and may be used for measuring the adhesion strength as described herein. Instruments for performing such measurements include the monotonic uniaxial tensile testing device provided by Bose® Biodynamic Test Instrument, Minnetonka, MN, for example by employing at a constant rate (e.g., 0.05 mm/sec) and continuously recording the load response (e.g., 200 measurements/sec) to the point of macroscopic failure, or the Avery Adhesive Test (AAT).

The term “port” is used in accordance with its plain ordinary meaning and refers to a designated entry or exit point on the device where fluids, gases, or other substances can be introduced into or removed from the microfluidic system. Ports are typically small and precise to accommodate the scaled-down dimensions of microfluidic channels and chambers. For example, the solid support may include an inlet port, that is, a port through which fluids (such as reagents, samples, or solvents) are introduced into the microfluidic device. The solid support may include an outlet port through which fluids exit the microfluidic device. In embodiments, the inlet and outlet ports are distinct and separate. In embodiments, the inlet port and the outlet ports are the same.

As used herein, the term “inlet” or “inlet port” refers to the location on a flow cell assembly where the reagents and fluids used for methods described herein enters the flow cell. As used herein, the term “outlet” or “outlet port” refers to the location on a flow cell assembly where the reagents and fluids used for methods described herein exits the flow cell after contacting the reaction chamber containing the cell or tissue to be analyzed.

As used herein, the term “resected” or “resection” is used in accordance with its plain and ordinary meaning and refers to removal of part or all of a tissue or an organ from a subject, typically through surgical removal.

### II. Compositions & Kits

In an aspect is provided a solid support. In embodiments, the solid support includes a polymer, wherein the polymer is attached to the solid support and includes a subunit having the formula:

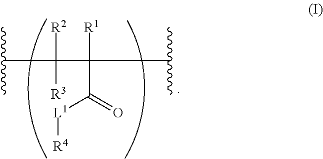

In embodiments, the polymer includes a plurality of subunits of formula (I). R¹ is hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. Rz is hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R³ is hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁴ is —N₃ or a protected amine (e.g., an amine moiety including a protecting group). In embodiments, R⁴ is —NR⁵R⁶. R⁵ is hydrogen, substituted or unsubstituted alkyl, substituted or unsubstituted heteroalkyl, or an amine protecting group. R⁶ is hydrogen, substituted or unsubstituted alkyl, or substituted or unsubstituted heteroalkyl, or an amine protecting group. L¹ is a covalent linker. In embodiments, a cell or tissue section is attached to the polymer. In embodiments, a plurality of cells is attached to the polymer.

In embodiments, R¹ is hydrogen, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, a substituted R¹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹ is substituted with a plurality of groups selected from substituent groups, size-limited substituent groups, and lower substituent groups; each substituent group, size-limited substituent group, and/or lower substituent group may optionally be different. In embodiments, when R¹ is substituted, it is substituted with at least one substituent group. In embodiments, when R¹ is substituted, it is substituted with at least one size-limited substituent group. In embodiments, when R¹ is substituted, it is substituted with at least one lower substituent group.

In embodiments, R¹ is hydrogen, or substituted or unsubstituted alkyl. In embodiments, R¹ is substituted or unsubstituted alkyl (e.g., C₁-C₈, C₁-C₆, C₁-C₄, or C₁-C₂) or substituted or unsubstituted heteroalkyl (e.g., 2 to 8 membered, 2 to 6 membered, 4 to 6 membered, 2 to 3 membered, or 4 to 5 membered). In embodiments, R¹ is unsubstituted alkyl. In embodiments, R¹ is hydrogen. In embodiments, R¹ is unsubstituted C₁-C₄ alkyl. In embodiments, R¹ is unsubstituted methyl. In embodiments, R¹ is unsubstituted ethyl. In embodiments, R¹ is unsubstituted propyl. In embodiments, R¹ is unsubstituted n-propyl. In embodiments, R¹ is unsubstituted isopropyl. In embodiments, R¹ is unsubstituted butyl. In embodiments, R¹ is unsubstituted n-butyl. In embodiments, R¹ is unsubstituted isobutyl. In embodiments, R¹ is unsubstituted tert-butyl. In embodiments, R¹ is substituted or unsubstituted 2 to 12 membered heteroalkyl.

In embodiments, R² is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R² is hydrogen, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, a substituted R² (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R² is substituted with a plurality of groups selected from substituent groups, size-limited substituent groups, and lower substituent groups; each substituent group, size-limited substituent group, and/or lower substituent group may optionally be different. In embodiments, when R² is substituted, it is substituted with at least one substituent group. In embodiments, when R² is substituted, it is substituted with at least one size-limited substituent group. In embodiments, when R² is substituted, it is substituted with at least one lower substituent group.

In embodiments, R² is hydrogen, or substituted or unsubstituted alkyl. In embodiments, R² is substituted or unsubstituted alkyl (e.g., C₁-C₈, C₁-C₆, C₁-C₄, or C₁-C₂) or substituted or unsubstituted heteroalkyl (e.g., 2 to 8 membered, 2 to 6 membered, 4 to 6 membered, 2 to 3 membered, or 4 to 5 membered). In embodiments, R² is unsubstituted alkyl. In embodiments, R² is hydrogen. In embodiments, R² is unsubstituted C₁-C₄ alkyl. In embodiments, R² is unsubstituted methyl. In embodiments, R² is unsubstituted ethyl. In embodiments, R² is unsubstituted propyl. In embodiments, R² is unsubstituted n-propyl. In embodiments, R² is unsubstituted isopropyl. In embodiments, R² is unsubstituted butyl. In embodiments, R² is unsubstituted n-butyl. In embodiments, R² is unsubstituted isobutyl. In embodiments, R² is unsubstituted tert-butyl. In embodiments, R² is substituted or unsubstituted 2 to 12 membered heteroalkyl.

In embodiments, R³ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R³ is hydrogen, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, a substituted R³ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R³ is substituted with a plurality of groups selected from substituent groups, size-limited substituent groups, and lower substituent groups; each substituent group, size-limited substituent group, and/or lower substituent group may optionally be different. In embodiments, when R³ is substituted, it is substituted with at least one substituent group. In embodiments, when R³ is substituted, it is substituted with at least one size-limited substituent group. In embodiments, when R³ is substituted, it is substituted with at least one lower substituent group.

In embodiments, R³ is hydrogen, or substituted or unsubstituted alkyl. In embodiments, R³ is substituted or unsubstituted alkyl (e.g., C₁-C₈, C₁-C₆, C₁-C₄, or C₁-C₂) or substituted or unsubstituted heteroalkyl (e.g., 2 to 8 membered, 2 to 6 membered, 4 to 6 membered, 2 to 3 membered, or 4 to 5 membered). In embodiments, R³ is unsubstituted alkyl. In embodiments, R³ is hydrogen. In embodiments, R³ is unsubstituted C₁-C₄ alkyl. In embodiments, R³ is unsubstituted methyl. In embodiments, R³ is unsubstituted ethyl. In embodiments, R³ is unsubstituted propyl. In embodiments, R³ is unsubstituted n-propyl. In embodiments, R³ is unsubstituted isopropyl. In embodiments, R³ is unsubstituted butyl. In embodiments, R³ is unsubstituted n-butyl. In embodiments, R³ is unsubstituted isobutyl. In embodiments, R³ is unsubstituted tert-butyl. In embodiments, R³ is substituted or unsubstituted 2 to 12 membered heteroalkyl.

In embodiments, R² and R³ are hydrogen.

In embodiments, R⁴ is —N₃ or a protected amine (e.g., a nitrogen atom attached to a protecting group). In embodiments, R⁴ is —NR⁵R⁶. In embodiments, R⁴ is —NHR⁶. In embodiments, R⁴ is —NH₂. In embodiments, R⁴ is —N₃.

In embodiments, R⁵ is hydrogen. In embodiments, R⁵ is substituted or unsubstituted alkyl, substituted or unsubstituted heteroalkyl, or an amine protecting group. In embodiments, R⁵ is hydrogen, substituted or unsubstituted alkyl (e.g., C₁-C₈, C₁-C₆, C₁-C₄, or C₁-C₂, or substituted or unsubstituted heteroalkyl (e.g., 2 to 8 membered, 2 to 6 membered, 4 to 6 membered, 2 to 3 membered, or 4 to 5 membered).

In embodiments, a substituted R⁵ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁵ is substituted with a plurality of groups selected from substituent groups, size-limited substituent groups, and lower substituent groups; each substituent group, size-limited substituent group, and/or lower substituent group may optionally be different. In embodiments, when R⁵ is substituted, it is substituted with at least one substituent group. In embodiments, when R⁵ is substituted, it is substituted with at least one size-limited substituent group. In embodiments, when R⁵ is substituted, it is substituted with at least one lower substituent group. In embodiments, R⁵ is unsubstituted alkyl. In embodiments, R⁵ is hydrogen. In embodiments, R⁵ is unsubstituted C₁-C₄ alkyl. In embodiments, R⁵ is unsubstituted methyl. In embodiments, R⁵ is unsubstituted ethyl. In embodiments, R⁵ is unsubstituted propyl. In embodiments, R⁵ is unsubstituted n-propyl. In embodiments, R⁵ is unsubstituted isopropyl. In embodiments, R⁵ is unsubstituted butyl. In embodiments, R⁵ is unsubstituted n-butyl. In embodiments, R⁵ is unsubstituted isobutyl. In embodiments, R⁵ is unsubstituted tert-butyl. In embodiments, R⁵ is substituted or unsubstituted 2 to 12 membered heteroalkyl.

In embodiments, R⁶ is hydrogen. In embodiments, R⁶ is substituted or unsubstituted alkyl, or substituted or unsubstituted heteroalkyl, or an amine protecting group. In embodiments, R⁶ is hydrogen. In embodiments, R⁶ is substituted or unsubstituted alkyl, substituted or unsubstituted heteroalkyl, or an amine protecting group. In embodiments, R⁶ is hydrogen, substituted or unsubstituted alkyl (e.g., C₁-C₈, C₁-C₆, C₁-C₄, or C₁-C₂), or substituted or unsubstituted heteroalkyl (e.g., 2 to 8 membered, 2 to 6 membered, 4 to 6 membered, 2 to 3 membered, or 4 to 5 membered).

In embodiments, a substituted R⁶ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁶ is substituted with a plurality of groups selected from substituent groups, size-limited substituent groups, and lower substituent groups; each substituent group, size-limited substituent group, and/or lower substituent group may optionally be different. In embodiments, when R⁶ is substituted, it is substituted with at least one substituent group. In embodiments, when R⁶ is substituted, it is substituted with at least one size-limited substituent group. In embodiments, when R⁶ is substituted, it is substituted with at least one lower substituent group. In embodiments, R⁶ is unsubstituted alkyl. In embodiments, R⁶ is hydrogen. In embodiments, R⁶ is unsubstituted C₁-C₄ alkyl. In embodiments, R⁶ is unsubstituted methyl. In embodiments, R⁶ is unsubstituted ethyl. In embodiments, R⁶ is unsubstituted propyl. In embodiments, R⁶ is unsubstituted n-propyl. In embodiments, R⁶ is unsubstituted isopropyl. In embodiments, R⁶ is unsubstituted butyl. In embodiments, R⁶ is unsubstituted n-butyl. In embodiments, R⁶ is unsubstituted isobutyl. In embodiments, R⁶ is unsubstituted tert-butyl. In embodiments, R⁶ is substituted or unsubstituted 2 to 12 membered heteroalkyl.

In embodiments, L¹ is L¹⁰¹-L¹⁰²-L¹⁰³, L¹⁰¹, L¹⁰², and L¹⁰³ are independently a bond, —S(O)₂—, —S(O)—, —S(O)₂NH—, —NH—, —O—, —S—, —SS—, —C(O)—, —C(O)NH—, —C(O)CH₂—, —NHC(O)—, —NH C(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L¹⁰¹ is R¹⁰¹-substituted or unsubstituted alkylene, R¹⁰¹-substituted or unsubstituted heteroalkylene, R¹⁰¹-substituted or unsubstituted cycloalkylene, R¹⁰¹-substituted or unsubstituted heterocycloalkylene, R¹⁰¹-substituted or unsubstituted arylene, or R¹⁰¹-substituted or unsubstituted heteroarylene. In embodiments, L¹⁰¹ is a bond. In embodiments, L¹⁰¹ is —O—. In embodiments, L¹⁰¹ is a —O—CH₂—CH₂—O—.

In embodiments, L¹⁰² is a bond, —C(O)O—, —NHC(O)—, —C(O)NH—, R¹⁰²-substituted or unsubstituted alkylene, R¹⁰²-substituted or unsubstituted heteroalkylene, R¹⁰²-substituted or unsubstituted cycloalkylene, R¹⁰²-substituted or unsubstituted heterocycloalkylene, R¹⁰²-substituted or unsubstituted arylene, or R¹⁰²-substituted or unsubstituted heteroarylene. In embodiments, L¹⁰² is a bond. In embodiments, L¹⁰² is —[CH₂—CH₂—O]m-, wherein m is as described herein (e.g., 6-12).

In embodiments, L¹⁰³ is a bond, —C(O)O—, —NHC(O)—, —C(O)NH—, R¹⁰³-substituted or unsubstituted alkylene, R¹⁰³-substituted or unsubstituted heteroalkylene, R¹⁰³-substituted or unsubstituted cycloalkylene, R¹⁰³-substituted or unsubstituted heterocycloalkylene, R¹⁰³-substituted or unsubstituted arylene, or R¹⁰³-substituted or unsubstituted heteroarylene. In embodiments, L¹⁰³ is a bond. In embodiments, L¹⁰³ is —CH₂—CH₂—.

R¹⁰¹, R¹⁰², and R¹⁰³ are independently oxo, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, —SF₅, —NH₃+, —SO₃, —OPO₃H, —SCN, —ONO₂, unsubstituted alkyl (e.g., C₁-C₂₀, C₁₀-C₂₀, C₁-C₈, C₁-C₆, or C₁-C₄), unsubstituted heteroalkyl (e.g., 2 to 20, 8 to 20, 2 to 10, 2 to 8, 2 to 6, or 2 to 4 membered), unsubstituted cycloalkyl (e.g., C₃-C₈, C₃-C₆, or C₅-C₆), unsubstituted heterocycloalkyl (e.g., 3 to 8, 3 to 6, or 5 to 6 membered), unsubstituted aryl (e.g., C₆-C₁₀, C₁₀, or phenyl), or unsubstituted heteroaryl (e.g., 5 to 10, 5 to 9, or 5 to 6 membered).

In embodiments, L¹⁰¹, L¹⁰², and L¹⁰³ independently include PEG. In embodiments, L¹⁰¹ is —O—, L¹⁰² is unsubstituted alkyl, and L¹⁰³ is

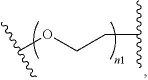

and n1 is an integer from 1 to 10.

In embodiments, L¹⁰¹, L¹⁰², and L¹⁰³ are independently a bond, —NH—, —O—, —S—, substituted or unsubstituted alkylene, or substituted or unsubstituted heteroalkylene. In embodiments, L¹⁰¹ is —NH— or —O—, and L¹⁰² is substituted or unsubstituted heteroalkylene. In embodiments, L¹⁰¹ is —O—, and L¹⁰² is unsubstituted 2 to 24 membered heteroalkylene.

In embodiments, L¹ is

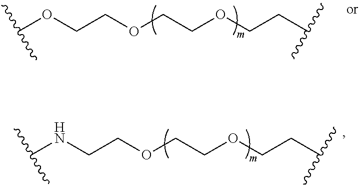

where m is an integer from 0 to 24. In embodiments, m is 0. In embodiments, m is 1. In embodiments, m is 2. In embodiments, m is 3. In embodiments, m is 4. In embodiments, m is 5. In embodiments, m is 6. In embodiments, m is 7. In embodiments, m is 8. In embodiments, m is 9. In embodiments, m is 10. In embodiments, m is 11. In embodiments, m is 12. In embodiments, m is 13. In embodiments, m is 14. In embodiments, m is 15. In embodiments, m is 16. In embodiments, m is 17. In embodiments, m is 18. In embodiments, m is 19. In embodiments, m is 20. In embodiments, m is 21. In embodiments, m is 22. In embodiments, m is 23. In embodiments, m is 24. In embodiments, m is an integer from 1 to 12. In embodiments, m is an integer from 2 to 12. In embodiments, m is an integer from 4 to 12. In embodiments, m is an integer from 6 to 12.

In embodiments, L¹ is

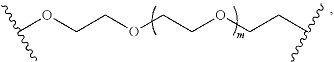

wherein m is an integer from 6 to 24. In embodiments, L¹ is

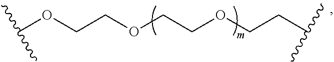

wherein m is an integer from 8 to 12.

In embodiments, the polymer includes a plurality of subunits having the formula:

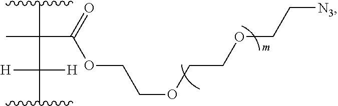

wherein m is an integer from 2 to 24. In embodiments, the polymer includes a plurality of subunits having the formula:

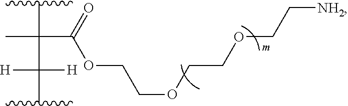

wherein m is an integer from 2 to 24.

In embodiments, the polymer includes one or more additional subunits to form a copolymer. In embodiments, the additional subunits have the formula

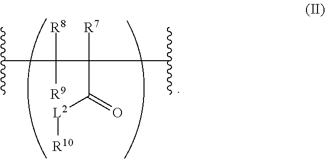

R⁷ is hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁸ is hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁹ is hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R¹⁰ is

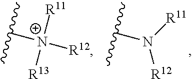

hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R¹¹ is hydrogen, substituted or unsubstituted alkyl, substituted or unsubstituted heteroalkyl. R¹² is hydrogen, substituted or unsubstituted alkyl, or substituted or unsubstituted heteroalkyl. R¹³ is hydrogen, substituted or unsubstituted alkyl, or substituted or unsubstituted heteroalkyl. L² is a covalent linker.

In embodiments, R⁷ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁷ is hydrogen, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, a substituted R⁷ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁷ is substituted with a plurality of groups selected from substituent groups, size-limited substituent groups, and lower substituent groups; each substituent group, size-limited substituent group, and/or lower substituent group may optionally be different. In embodiments, when R⁷ is substituted, it is substituted with at least one substituent group. In embodiments, when R⁷ is substituted, it is substituted with at least one size-limited substituent group. In embodiments, when R⁷ is substituted, it is substituted with at least one lower substituent group.

In embodiments, R⁷ is hydrogen, or substituted or unsubstituted alkyl. In embodiments, R⁷ is substituted or unsubstituted alkyl (e.g., C₁-C₈, C₁-C₆, C₁-C₄, or C₁-C₂) or substituted or unsubstituted heteroalkyl (e.g., 2 to 8 membered, 2 to 6 membered, 4 to 6 membered, 2 to 3 membered, or 4 to 5 membered). In embodiments, R⁷ is unsubstituted alkyl. In embodiments, R⁷ is hydrogen. In embodiments, R⁷ is unsubstituted C₁-C₄ alkyl. In embodiments, R⁷ is unsubstituted methyl. In embodiments, R⁷ is unsubstituted ethyl. In embodiments, R⁷ is unsubstituted propyl. In embodiments, R⁷ is unsubstituted n-propyl. In embodiments, R⁷ is unsubstituted isopropyl. In embodiments, R⁷ is unsubstituted butyl. In embodiments, R⁷ is unsubstituted n-butyl. In embodiments, R⁷ is unsubstituted isobutyl. In embodiments, R⁷ is unsubstituted tert-butyl. In embodiments, R⁷ is substituted or unsubstituted 2 to 12 membered heteroalkyl.

In embodiments, R⁸ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁸ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R⁸ is hydrogen, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, a substituted R⁸ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁸ is substituted with a plurality of groups selected from substituent groups, size-limited substituent groups, and lower substituent groups; each substituent group, size-limited substituent group, and/or lower substituent group may optionally be different. In embodiments, when R⁸ is substituted, it is substituted with at least one substituent group. In embodiments, when R⁸ is substituted, it is substituted with at least one size-limited substituent group. In embodiments, when R⁸ is substituted, it is substituted with at least one lower substituent group.

In embodiments, R⁸ is hydrogen or substituted or unsubstituted alkyl. In embodiments, R⁸ is substituted or unsubstituted alkyl (e.g., C₁-C₈, C₁-C₆, C₁-C₄, or C₁-C₂) or substituted or unsubstituted heteroalkyl (e.g., 2 to 8 membered, 2 to 6 membered, 4 to 6 membered, 2 to 3 membered, or 4 to 5 membered). In embodiments, R⁸ is unsubstituted alkyl. In embodiments, R⁸ is hydrogen. In embodiments, R⁸ is unsubstituted C₁-C₄ alkyl. In embodiments, R⁸ is unsubstituted methyl. In embodiments, R⁸ is unsubstituted ethyl. In embodiments, R⁸ is unsubstituted propyl. In embodiments, R⁸ is unsubstituted n-propyl. In embodiments, R⁸ is unsubstituted isopropyl. In embodiments, R⁸ is unsubstituted butyl. In embodiments, R⁸ is unsubstituted n-butyl. In embodiments, R⁸ is unsubstituted isobutyl. In embodiments, R⁸ is unsubstituted tert-butyl. In embodiments, R⁸ is substituted or unsubstituted 2 to 12 membered heteroalkyl.

In embodiments, R⁹ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁹ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R⁹ is hydrogen, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, a substituted R⁹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁹ is substituted with a plurality of groups selected from substituent groups, size-limited substituent groups, and lower substituent groups; each substituent group, size-limited substituent group, and/or lower substituent group may optionally be different. In embodiments, when R⁹ is substituted, it is substituted with at least one substituent group. In embodiments, when R⁹ is substituted, it is substituted with at least one size-limited substituent group. In embodiments, when R⁹ is substituted, it is substituted with at least one lower substituent group.

In embodiments, R⁹ is hydrogen or substituted or unsubstituted alkyl. In embodiments, R⁹ is substituted or unsubstituted alkyl (e.g., C₁-C₈, C₁-C₆, C₁-C₄, or C₁-C₂) or substituted or unsubstituted heteroalkyl (e.g., 2 to 8 membered, 2 to 6 membered, 4 to 6 membered, 2 to 3 membered, or 4 to 5 membered). In embodiments, R⁹ is unsubstituted alkyl. In embodiments, R⁹ is hydrogen. In embodiments, R⁹ is unsubstituted C₁-C₄ alkyl. In embodiments, R⁹ is unsubstituted methyl. In embodiments, R⁹ is unsubstituted ethyl. In embodiments, R⁹ is unsubstituted propyl. In embodiments, R⁹ is unsubstituted n-propyl. In embodiments, R⁹ is unsubstituted isopropyl. In embodiments, R⁹ is unsubstituted butyl. In embodiments, R⁹ is unsubstituted n-butyl. In embodiments, R⁹ is unsubstituted isobutyl. In embodiments, R⁹ is unsubstituted tert-butyl. In embodiments, R⁹ is substituted or unsubstituted 2 to 12 membered heteroalkyl.

In embodiments, R¹⁰ is

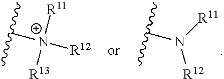

In embodiments, R¹⁰ is

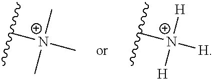

In embodiments, R¹⁰ is

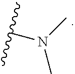 [SMILES: *N(C)C]

In embodiments, R¹⁰ is

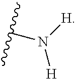

In embodiments, R¹⁰ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹⁰ is hydrogen. In embodiments, R¹⁰ is substituted or unsubstituted alkyl or substituted or unsubstituted heteroalkyl. In embodiments, R¹⁰ is substituted or unsubstituted alkyl, substituted or unsubstituted heteroalkyl, or an amine protecting group. In embodiments, R¹⁰ is hydrogen, substituted or unsubstituted alkyl (e.g., C₁-C₈, C₁-C₆, C₁-C₄, or C₁-C₂), or substituted or unsubstituted heteroalkyl (e.g., 2 to 8 membered, 2 to 6 membered, 4 to 6 membered, 2 to 3 membered, or 4 to 5 membered).

In embodiments, a substituted R¹⁰ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁰ is substituted with a plurality of groups selected from substituent groups, size-limited substituent groups, and lower substituent groups; each substituent group, size-limited substituent group, and/or lower substituent group may optionally be different. In embodiments, when R¹⁰ is substituted, it is substituted with at least one substituent group. In embodiments, when R¹⁰ is substituted, it is substituted with at least one size-limited substituent group. In embodiments, when R¹⁰ is substituted, it is substituted with at least one lower substituent group. In embodiments, R¹⁰ is unsubstituted alkyl. In embodiments, R¹⁰ is hydrogen. In embodiments, R¹⁰ is unsubstituted C₁-C₄ alkyl. In embodiments, R¹⁰ is unsubstituted methyl. In embodiments, R¹⁰ is unsubstituted ethyl. In embodiments, R¹⁰ is unsubstituted propyl. In embodiments, R¹⁰ is unsubstituted n-propyl. In embodiments, R¹⁰ is unsubstituted isopropyl. In embodiments, R¹⁰ is unsubstituted butyl. In embodiments, R¹⁰ is unsubstituted n-butyl. In embodiments, R¹⁰ is unsubstituted isobutyl. In embodiments, R¹⁰ is unsubstituted tert-butyl. In embodiments, R¹⁰ is substituted or unsubstituted 2 to 12 membered heteroalkyl.

In embodiments, R¹¹ is hydrogen. In embodiments, R¹¹ is substituted or unsubstituted alkyl or substituted or unsubstituted heteroalkyl. In embodiments, R¹¹ is substituted or unsubstituted alkyl, substituted or unsubstituted heteroalkyl, or an amine protecting group. In embodiments, R¹¹ is hydrogen, substituted or unsubstituted alkyl (e.g., C₁-C₈, C₁-C₆, C₁-C₄, or C₁-C₂, or substituted or unsubstituted heteroalkyl (e.g., 2 to 8 membered, 2 to 6 membered, 4 to 6 membered, 2 to 3 membered, or 4 to 5 membered).

In embodiments, a substituted R¹¹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹¹ is substituted with a plurality of groups selected from substituent groups, size-limited substituent groups, and lower substituent groups; each substituent group, size-limited substituent group, and/or lower substituent group may optionally be different. In embodiments, when R¹¹ is substituted, it is substituted with at least one substituent group. In embodiments, when R¹¹ is substituted, it is substituted with at least one size-limited substituent group. In embodiments, when R¹¹ is substituted, it is substituted with at least one lower substituent group. In embodiments, R¹¹ is unsubstituted alkyl. In embodiments, R¹¹ is hydrogen. In embodiments, R¹¹ is unsubstituted C₁-C₄ alkyl. In embodiments, R¹¹ is unsubstituted methyl. In embodiments, R¹¹ is unsubstituted ethyl. In embodiments, R¹¹ is unsubstituted propyl. In embodiments, R¹¹ is unsubstituted n-propyl. In embodiments, R¹¹ is unsubstituted isopropyl. In embodiments, R¹¹ is unsubstituted butyl. In embodiments, R¹¹ is unsubstituted n-butyl. In embodiments, R¹¹ is unsubstituted isobutyl. In embodiments, R¹¹ is unsubstituted tert-butyl. In embodiments, R¹¹ is substituted or unsubstituted 2 to 12 membered heteroalkyl.

In embodiments, R¹² is hydrogen. In embodiments, R¹² is substituted or unsubstituted alkyl or substituted or unsubstituted heteroalkyl. In embodiments, R¹² is substituted or unsubstituted alkyl, substituted or unsubstituted heteroalkyl, or an amine protecting group. In embodiments, R¹² is hydrogen, substituted or unsubstituted alkyl (e.g., C₁-C₈, C₁-C₆, C₁-C₄, or C₁-C₂), or substituted or unsubstituted heteroalkyl (e.g., 2 to 8 membered, 2 to 6 membered, 4 to 6 membered, 2 to 3 membered, or 4 to 5 membered).

In embodiments, a substituted R¹² (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹² is substituted with a plurality of groups selected from substituent groups, size-limited substituent groups, and lower substituent groups; each substituent group, size-limited substituent group, and/or lower substituent group may optionally be different. In embodiments, when R¹² is substituted, it is substituted with at least one substituent group. In embodiments, when R¹² is substituted, it is substituted with at least one size-limited substituent group. In embodiments, when R¹² is substituted, it is substituted with at least one lower substituent group. In embodiments, R¹² is unsubstituted alkyl. In embodiments, R¹² is hydrogen. In embodiments, R¹² is unsubstituted C₁-C₄ alkyl. In embodiments, R¹² is unsubstituted methyl. In embodiments, R¹² is unsubstituted ethyl. In embodiments, R¹² is unsubstituted propyl. In embodiments, R¹² is unsubstituted n-propyl. In embodiments, R¹² is unsubstituted isopropyl. In embodiments, R¹² is unsubstituted butyl. In embodiments, R¹² is unsubstituted n-butyl. In embodiments, R¹² is unsubstituted isobutyl. In embodiments, R¹² is unsubstituted tert-butyl. In embodiments, R¹² is substituted or unsubstituted 2 to 12 membered heteroalkyl.

In embodiments, R¹³ is hydrogen. In embodiments, R¹³ is substituted or unsubstituted alkyl or substituted or unsubstituted heteroalkyl. In embodiments, R¹³ is substituted or unsubstituted alkyl, substituted or unsubstituted heteroalkyl, or an amine protecting group. In embodiments, R¹³ is hydrogen, substituted or unsubstituted alkyl (e.g., C₁-C₈, C₁-C₆, C₁-C₄, or C₁-C₂), or substituted or unsubstituted heteroalkyl (e.g., 2 to 8 membered, 2 to 6 membered, 4 to 6 membered, 2 to 3 membered, or 4 to 5 membered).

In embodiments, a substituted R¹³ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹³ is substituted with a plurality of groups selected from substituent groups, size-limited substituent groups, and lower substituent groups; each substituent group, size-limited substituent group, and/or lower substituent group may optionally be different. In embodiments, when R¹³ is substituted, it is substituted with at least one substituent group. In embodiments, when R¹³ is substituted, it is substituted with at least one size-limited substituent group. In embodiments, when R¹³ is substituted, it is substituted with at least one lower substituent group. In embodiments, R¹³ is unsubstituted alkyl. In embodiments, R¹³ is hydrogen. In embodiments, R¹³ is unsubstituted C₁-C₄ alkyl. In embodiments, R¹³ is unsubstituted methyl. In embodiments, R¹³ is unsubstituted ethyl. In embodiments, R¹³ is unsubstituted propyl. In embodiments, R¹³ is unsubstituted n-propyl. In embodiments, R¹³ is unsubstituted isopropyl. In embodiments, R¹³ is unsubstituted butyl. In embodiments, R¹³ is unsubstituted n-butyl. In embodiments, R¹³ is unsubstituted isobutyl. In embodiments, R¹³ is unsubstituted tert-butyl. In embodiments, R¹³ is substituted or unsubstituted 2 to 12 membered heteroalkyl.

In embodiments, L¹ is L²⁰¹-L²⁰²-L²⁰³. L²⁰¹, L²⁰², and L²⁰³ are independently a bond, —S(O)₂—, —S(O)—, —S(O)₂NH—, —NH—, —O—, —S—, —SS—, —C(O)—, —C(O)NH—, —C(O)CH₂—, —NHC(O)—, —NH C(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L²⁰¹ is R²⁰¹-substituted or unsubstituted alkylene, R²⁰¹-substituted or unsubstituted heteroalkylene, R²⁰¹-substituted or unsubstituted cycloalkylene, R²⁰¹-substituted or unsubstituted heterocycloalkylene, R²⁰¹-substituted or unsubstituted arylene, or R²⁰¹-substituted or unsubstituted heteroarylene. In embodiments, L²⁰¹ is a bond.

In embodiments, L²⁰² is a bond, —C(O)O—, —NHC(O)—, —C(O)NH—, R²⁰²-substituted or unsubstituted alkylene, R²⁰²-substituted or unsubstituted heteroalkylene, R²⁰²-substituted or unsubstituted cycloalkylene, R²⁰²-substituted or unsubstituted heterocycloalkylene, R²⁰²-substituted or unsubstituted arylene, or R²⁰²-substituted or unsubstituted heteroarylene. In embodiments, L²⁰² is a bond.

In embodiments, L²⁰³ is a bond, —C(O)O—, —NHC(O)—, —C(O)NH—, R²⁰³-substituted or unsubstituted alkylene, R²⁰³-substituted or unsubstituted heteroalkylene, R²⁰³-substituted or unsubstituted cycloalkylene, R²⁰³-substituted or unsubstituted heterocycloalkylene, R²⁰³-substituted or unsubstituted arylene, or R²⁰³-substituted or unsubstituted heteroarylene. In embodiments, L²⁰³ is a bond.

R²⁰¹, R²⁰², and R²⁰³ are independently oxo, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, —SF₅, —NH₃+, —SO₃, —OPO₃H, —SCN, —ONO₂, unsubstituted alkyl (e.g., C₁-C₂₀, C₁₀-C₂₀, C₁-C₈, C₁-C₆, or C₁-C₄), unsubstituted heteroalkyl (e.g., 2 to 20, 8 to 20, 2 to 10, 2 to 8, 2 to 6, or 2 to 4 membered), unsubstituted cycloalkyl (e.g., C₃-C₈, C₃-C₆, or C₅-C₆), unsubstituted heterocycloalkyl (e.g., 3 to 8, 3 to 6, or 5 to 6 membered), unsubstituted aryl (e.g., C₆-C₁₀, C₁₀, or phenyl), or unsubstituted heteroaryl (e.g., 5 to 10, 5 to 9, or 5 to 6 membered).

In embodiments, the polymer includes additional polymerized subunits having the formula:

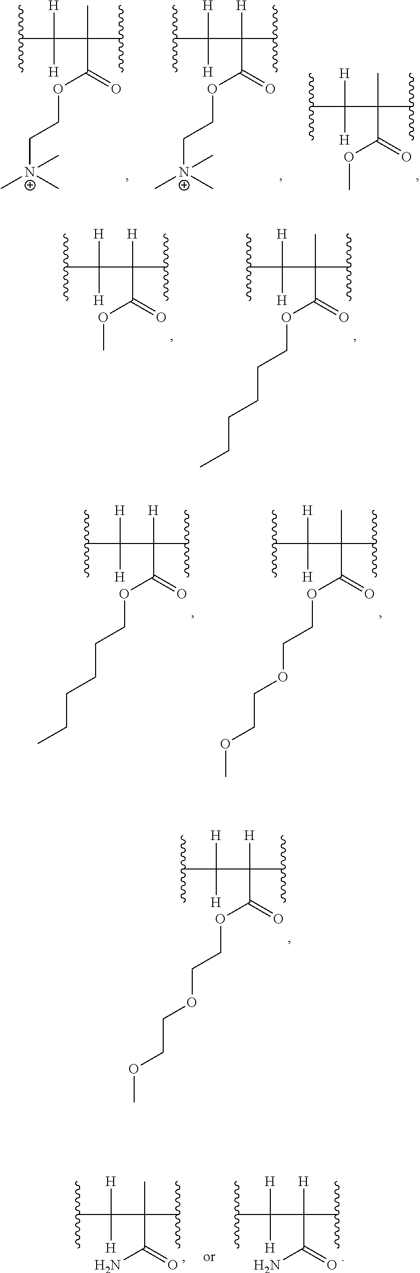

In embodiments, the polymer includes polymerized units of N-Hydroxyethyl acrylamide (HEAA), 2-Hydroxyethyl Acrylate (HEA), 2-Hydroxyethyl methacrylate (HEMA), Acrylic acid (AA), N-vinyl pyrrolidone (NVP), N-isopropylacrylamide (NIPAM), acrylamide, or poly(ethylene glycol) methacrylate (PEGMA). In embodiments, the polymer includes polymerized units of Glycidyl methacrylate (GMA), Dopamine methacrylate (DMA), Acrylic acid N-hydroxysuccinimide ester (AA-NHS), 2-Isocyanatoethyl Acrylate (ICEA), N-(3-aminopropyl)methacrylamide hydrochloride (APMA), 2-hydroxyethyl methacrylate (HEMA) modified with N-hydroxysuccinimide (HEMA-NHS), Epoxypropyl Methacrylate (EPMA), Glycidyl Acrylate (GA), Glycidyl Ethacrylate (GEA), or 3,4-Epoxybutyl Methacrylate (EBMA). In embodiments, the polymer includes polymerized units of [2-(Acryloyloxy)ethyl]trimethylammonium chloride (AETA), methacryloxypropyl trimethyl ammonium chloride (MPTA), dimethylaminoethyl methacrylate (DMAEMA), trimethylammonium ethyl methacrylate chloride (TMAEMC), methacryloxyethyltrimethyl ammonium chloride (METMAC), allyltrimethyl ammonium chloride (ATMAC), or vinylbenzyl trimethyl ammonium chloride (VBTMAC). In embodiments, the polymer is a polymer as described herein.

In embodiments, the tissue section is embedded in an embedding material, for example an embedding material including paraffin wax, polyepoxide polymer, polyacrylic polymer, agar, gelatin, celloidin, cryogel, optimal cutting temperature (OCT) composition, glycols, or a combination thereof. In embodiments, the tissue section includes a thickness of about 1 μm to about 20 μm. In embodiments, the tissue includes a thickness of about 1 μm to about 10 μm. In embodiments, the tissue includes a thickness of about 2 μm to about 3 μm. In embodiments, the tissue includes a thickness of about 4 μm to about 6 μm. In embodiments, the tissue includes a thickness of about 4 μm. In embodiments, the tissue includes a thickness of about 5 μm. In embodiments, the tissue includes a thickness of about 6 μm. In embodiments, the tissue includes a thickness of about 7 μm. In embodiments, the tissue includes a thickness of about 8 μm. In embodiments, the tissue includes a thickness of about 9 μm. In embodiments, the tissue includes a thickness of about 10 μm.

In embodiments, the tissue section includes a tissue or a cell (e.g., a plurality of cells such as blood cells). In embodiments, the tissue section includes one or more cells. In embodiments, the tissue section is embedded in an embedding material including paraffin wax, polyepoxide polymer, polyacrylic polymer, agar, gelatin, celloidin, cryogel, optimal cutting temperature (OCT) compositions, glycols, or a combination thereof. In embodiments, the tissue section is embedded in an embedding material including paraffin wax. In embodiments, the tissue section is embedded in an embedding material including a polyepoxide polymer. In embodiments, the tissue section is embedded in an embedding material including polyacrylic polymer. In embodiments, the tissue section is embedded in an embedding material including agar. In embodiments, the tissue section is embedded in an embedding material including gelatin. In embodiments, the tissue section is embedded in an embedding material including celloidin. In embodiments, the tissue section is embedded in an embedding material including a cryogel. In embodiments, the tissue section is embedded in an embedding material including an optimal cutting temperature (OCT) composition. In embodiments, the tissue section is embedded in an embedding material including one or more glycols. Tissue sections may be obtained from a subject by any means known and available in the art. In embodiments, a tissue section, e.g., a tumor tissue sample, is obtained from a subject by fine needle aspiration, core needle biopsy, stereotactic core needle biopsy, vacuum-assisted core biopsy, or surgical biopsy. In embodiments, the surgical biopsy is an incisional biopsy, which removes only part of the suspicious area.

In embodiments, the solid support includes an IR reflective coating. In embodiments, the IR reflective coating is attached to the solid support. In embodiments, the IR reflective coating is attached to the solid support, wherein the IR reflective coating is in contact with the polymer described herein. In embodiments, the IR reflective coating includes metal oxides. In embodiments, the IR reflective coating includes titanium dioxide, zinc oxide, tin oxide, tantalum pentoxide, silicon dioxide, indium tin oxide, silver-based coating, ceramic-based coating or a combination thereof. In embodiments, the IR reflective coating includes SiO₂, TiO₂, Al₂O₃ and Ta₂O₅ and fluorides such as MgF₂, LaF₃ and AlF₃. In embodiments, the IR reflective coating includes tantalum pentoxide (Ta₂O₅) and silicon dioxide (SiO₂). In embodiments, the infrared (IR) reflective coating includes one or more layers of silicon dioxide (SiO₂) and tantalum pentoxide (Ta₂O₅). In embodiments, the infrared (IR) reflective coating includes alternating layers of silicon dioxide (SiO₂) and tantalum pentoxide (Ta₂O₅), wherein the layer of silicon dioxide (SiO₂) is in direct or indirect contact with the polymer (e.g., the polymer described herein). In embodiments, the infrared (IR) reflective coating includes alternating layers of silicon dioxide (SiO₂) and tantalum pentoxide (Ta₂O₅), wherein the layer of tantalum pentoxide (Ta₂O₅) is in direct or indirect contact with the polymer (e.g., the polymer described herein).

In embodiments, the IR reflective coating reflects near-infrared radiation (NIR). In embodiments, the IR reflective coating reflects mid- or far-infrared radiation. In embodiments, the IR reflective coating reflects wavelengths greater than 750 nm. In embodiments, the IR reflective coating reflects wavelengths greater than 760 nm. In embodiments, the IR reflective coating reflects wavelengths greater than 770 nm. In embodiments, the IR reflective coating reflects wavelengths greater than 780 nm. In embodiments, the IR reflective coating reflects wavelengths greater than 790 nm. In embodiments, the IR reflective coating reflects wavelengths greater than 800 nm. In embodiments, the IR reflective coating reflects wavelengths from about 750 nm to 1,000 μm. In embodiments, the infrared (IR) reflective coating includes one or more layers of silicon dioxide (SiO₂) and tantalum pentoxide (Ta₂O₅). A multilayer configuration leverages the distinct optical properties of both materials to enhance the IR reflectivity. Silicon dioxide, known for its low refractive index, and tantalum pentoxide, recognized for its high refractive index, are alternately layered to create a stack that exhibits high reflectance in the infrared spectrum. The alternating layers of SiO₂ and Ta₂O₅ result in constructive interference of light at specific wavelengths, thereby enhancing the IR reflective capability of the coating. The number and thickness of these layers can be tailored to target specific wavelengths within the IR range, or permitting a certain percentage of radiation to transmit. For example, the IR reflective coating may reflect 2-3%, 2-6%, or 2 to 10% of the total IR radiation, and it absorbs or transmits the remaining IR radiation (e.g., greater than about 90% of the IR radiation). In embodiments, the IR reflective coating reflects about 1%, about 2%, about 3%, about 4%, about 5%, about 6%, about 7%, about 8%, about 9%, or about 10% of the total IR radiation.

In embodiments, the IR reflective coating aids autofocus mechanisms in optical instruments (e.g., fluorescence microscopy instruments) to provide consistent signal across various z-heights (e.g., the depth of an image). In embodiments, the IR reflective coating increases the amount of light reflected to the autofocus sensor to provide consistent signal across various z-heights. In embodiments, the IR reflective coating improves the signal to noise ratio of an image acquired by an optical instrument.

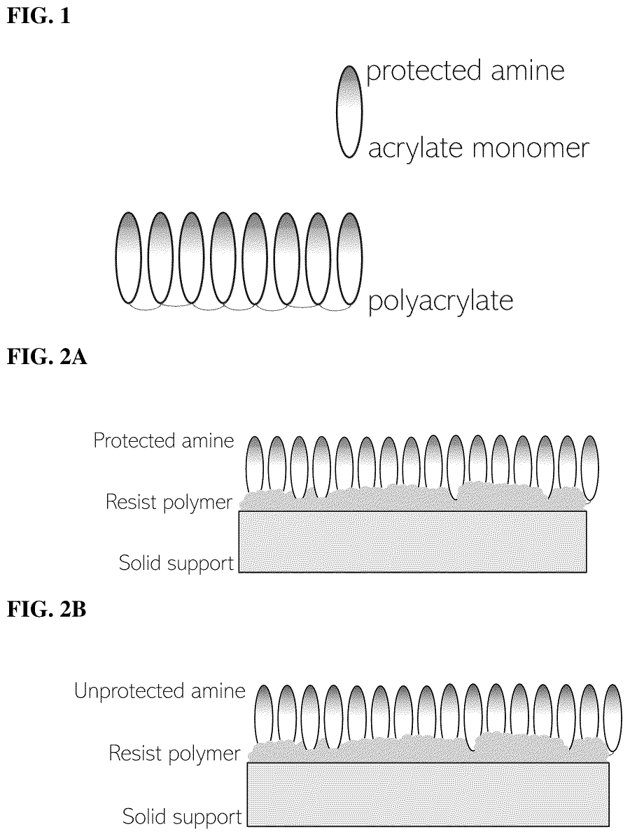

In embodiments, the solid support further includes a resist, wherein the resist is between the polymer and the solid support. See for example, FIG. 2A. In embodiments, the resist is a polydimethylsiloxane (PDMS), poly(methyl methacrylate) (PMMA), cyclic olefin copolymer (COC), silsesquioxane resist, an epoxy-based polymer resist, poly(vinylpyrrolidone-vinyl acrylic acid) copolymer resist, an Off-stoichiometry thiol-enes (OSTE) resist, amorphous fluoropolymer resist, a crystalline fluoropolymer resist, polysiloxane resist, SU-8 resist, or an organically modified ceramic polymer resist. In embodiments, the resist is an organically modified ceramic polymer resist.

In embodiments, the polymer attached to the solid support is a crosslinked polymer matrix. In embodiments, the polymer is a photoresist, wherein the photoresist is a polydimethylsiloxane (PDMS), poly(methyl methacrylate) (PMMA), cyclic olefin copolymer (COC), silsesquioxane resist, an epoxy-based polymer resist, poly(vinylpyrrolidone-vinyl acrylic acid) copolymer resist, an Off-stoichiometry thiol-enes (OSTE) resist, amorphous fluoropolymer resist, a crystalline fluoropolymer resist, polysiloxane resist, or an organically modified ceramic polymer resist. In embodiments, the polymer attached to the solid support includes acrylate silanes and polyamines. In embodiments, the polymer attached to the solid support includes methacrylic acid N-hydroxysuccinimide ester (NHS-MA). In embodiments, the polymer attached to the solid support includes (3-aminopropyl)triethoxysilane (APTES). In embodiments, the polymer attached to the solid support includes a copolymer of (3-aminopropyl)triethoxysilane (APTES) and methacrylic acid N-hydroxysuccinimide ester (NHS-MA).

The photoresist (alternatively referred to as a resist) is an active material layer that can be patterned by selective exposure and must “resist” chemical/physical attach of the underlying substrate. A photoresist is a light-sensitive polymer material used to form a patterned coating on a surface. The process begins by coating a substrate (e.g., a glass substrate) with a light-sensitive organic material. A mask with the desired pattern is used to block light so that only unmasked regions of the material will be exposed to light. In the case of a positive photoresist, the photo-sensitive material is degraded by light and a suitable solvent will dissolve away the regions that were exposed to light, leaving behind a coating where the mask was placed. In the case of a negative photoresist, the photosensitive material is strengthened (either polymerized or cross-linked) by light, and a suitable solvent will dissolve away only the regions that were not exposed to light, leaving behind a coating in areas where the mask was not placed. In embodiments, the solid support includes an epoxy-based photoresist (e.g., SU-8, SU-8 2000, SU-8 3000, SU-8 GLM2060). In embodiments, the solid support includes a negative photoresist. Negative refers to a photoresist whereby the parts exposed to UV become cross-linked (i.e., immobilized), while the remainder of the polymer remains soluble and can be washed away during development.

In embodiments, the solid support includes a glass substrate having a surface coated in silsesquioxane resist (e.g., polyhedral oligosilsesquioxanemethacrylate (POSS)), an epoxy-based polymer resist (e.g., SU-8 as described in U.S. Pat. No. 4,882,245), poly(vinylpyrrolidone-vinyl acrylic acid) copolymer resist (e.g., as described in U.S. Pat. No. 7,467,632), or novolaks resist, bisazides resist, or a combination thereof (e.g., as described in U.S. Pat. No. 4,970,276). In embodiments, the resist is removed prior to loading.

A “resist” as used herein is used in accordance with its ordinary meaning in the art of lilthography and refers to a polymer matrix (e.g., a polymer network). In embodiments, the photoresist is a silsesquioxane resist. In embodiments, the photoresist is an epoxy-based polymer resist. In embodiments, the photoresist is a poly(vinylpyrrolidone-vinyl acrylic acid) copolymer resist. In embodiments, the photoresist is an Off-stoichiometry thiol-enes (OSTE) resist. In embodiments, the solid support includes a Hydrogen Silsesquioxane (HSQ) polymer (e.g., HSQ resist). In embodiments, the photoresist is an amorphous fluoropolymer resist. In embodiments, the photoresist is a crystalline fluoropolymer resist. In embodiments, the photoresist is a polysiloxane resist. In embodiments, the photoresist is an organically modified ceramic polymer resist. In embodiments, the photoresist includes polymerized alkoxysilyl methacrylate polymers and metal oxides (e.g., SiO₂, ZrO, MgO, Al₂O₃, TiO₂ or Ta₂O₅). In embodiments, the photoresist includes polymerized alkoxysilyl acrylate polymers and metal oxides (e.g., SiO₂, ZrO, MgO, Al₂O₃, TiO₂ or Ta₂O₅). In embodiments, the photoresist includes metal atoms, such as Si, Zr, Mg, Al, Ti or Ta atoms.

In embodiments, the solid support includes a resist (e.g., a nanoimprint lithography (NIL) resist). Nanoimprint resists can include thermal curable materials (e.g., thermoplastic polymers), and/or UV-curable polymers. In embodiments, the solid support is generated by pressing a transparent mold possessing the pattern of interest (e.g., the pattern of wells) into photo-curable liquid film, followed by solidifying the liquid materials via a UV light irradiation. Typical UV-curable resists have low viscosity, low surface tension, and suitable adhesion to the glass substrate. For example, the solid support surface is coated in an organically modified ceramic polymer (ORMOCER®, registered trademark of Fraunhofer-Gesellschaft zur Förderung der angewandten Forschung e. V. in Germany). Organically modified ceramics contain organic side chains attached to an inorganic siloxane backbone. Several ORMOCER® polymers are now provided under names such as “Ormocore”, “Ormoclad” and “Ormocomp” by Micro Resist Technology GmbH. In embodiments, the solid support includes a resist as described in Haas et al Volume 351, Issues 1-2, 30 Aug. 1999, Pages 198-203, US 2015/0079351A1, US 2008/0000373, US 2010/0160478, or U.S. Pat. No. 10,268,096 B2, each of which is incorporated herein by reference. In embodiments, the solid support surface is coated in an organically modified ceramic polymer including (ORMOCER®, registered trademark of Fraunhofer-Gesellschaft zur Förderung der angewandten Forschung e. V. in Germany). In embodiments, the solid support surface is coated in an organically modified ceramic polymer wherein the organically modified ceramic polymer includes an inorganic-organic hybrid polymer that includes Si—O bonds. In embodiments, the solid support surface is coated in an organically modified ceramic polymer wherein the organically modified ceramic polymer includes an inorganic-organic hybrid polymer that includes Si—C bonds. In embodiments, the solid support surface is coated in an organically modified ceramic polymer wherein the organically modified ceramic polymer includes free acrylate moieties. In embodiments, the polymer is an organically modified ceramic polymer wherein the organically modified ceramic polymer includes an inorganic-organic hybrid polymer that includes Si—O bonds. In embodiments, polymer is an organically modified ceramic polymer wherein the organically modified ceramic polymer includes an inorganic-organic hybrid polymer that includes Si—C bonds. In embodiments, the polymer is an organically modified ceramic polymer wherein the organically modified ceramic polymer includes free acriate moieties. In embodiments, the polymer contains organically crosslinked heteropolysiloxane moieties.

In embodiments, the polymer attached to the solid support includes a plurality of particles. In embodiments, the particle is a solid particle. In embodiments, the particle is rigid and includes a shape. In embodiments, the particle is substantially spherical. In embodiments, the particle is substantially cuboidal. In embodiments, the particle is not an emulsion or droplet. In embodiments, the particle is a functionalized particle including pluralities of fluorescent moieties. In embodiments, the particle is a functionalized particle including pluralities of two different fluorescent moieties on its surface. In embodiments, the particle has a silica core. In embodiments, the particle has a polystyrene core. In embodiments, the particle has a gold core. In embodiments, the particle has a metal oxide core. In embodiments, the particle has an iron oxide core. In embodiments, the particle has a core that can be manipulated using magnetic fields. In embodiments, the particle has a nickel core. In embodiments, the particle has a cobalt core. In embodiments, the particle has a core with reflective properties. In embodiments, the particle has a silver core. In embodiments, the particle includes a polymer shell surrounding the particle core (e.g., a polymer shell that is attached to the particle core), wherein the polymer shell includes bioconjugate reactive moieties. In embodiments, the particle includes a polymer shell surrounding the particle core (e.g., a polymer shell that is attached to the particle core), wherein the polymer shell includes azide moieties. In embodiments, a fluorescent moiety is covalently attached to the polymer shell surrounding the particle core via a bioconjugate linker. In embodiments, the fluorescent moiety including a reactive bioconjugate moiety is allowed to contact the polymer shell surrounding the particle and form a bioconjugate linker, thereby covalently immobilizing the fluorescent moiety to the particle. In embodiments, a plurality of fluorescent moieties including reactive bioconjugate moieties are allowed to contact the polymer shell surrounding the particle and form a bioconjugate linker, thereby covalently immobilizing the fluorescent moiety to the particle. In embodiments, the particle is a fluorescent particle.

In embodiments, the particles attached to the polymer aids calibration of optical instruments used herein (e.g., fluorescence microscopy instruments). In embodiments, the particles used herein emit fluorescence at known wavelengths, which aids the calibration of fluorescence detection channels on optical instruments used herein. In embodiments, the particles used herein aids the testing the image quality and spatial resolution across different z-heights (e.g., depth of an image acquired of a tissue section described herein).

In embodiments, the average longest dimension of the particle is from about 100 nm to about 3000 nm. In embodiments, the average longest dimension of the particle is from about 200 nm to about 2900 nm. In embodiments, the average longest dimension of the particle is from about 300 nm to about 2800 nm. In embodiments, the average longest dimension of the particle is from about 400 nm to about 2700 nm. In embodiments, the average longest dimension of the particle is from about 500 nm to about 2600 nm. In embodiments, the average longest dimension of the particle is from about 600 nm to about 2500 nm. In embodiments, the average longest dimension of the particle is from about 700 nm to about 2400 nm. In embodiments, the average longest dimension of the particle is from about 800 nm to about 2300 nm. In embodiments, the average longest dimension of the particle is from about 900 nm to about 2200 nm. In embodiments, the average longest dimension of the particle is from about 1000 nm to about 2100 nm. In embodiments, the average longest dimension of the particle is from about 900 nm to about 2000 nm. In embodiments, the average longest dimension of the particle is from about 150 nm to about 600 nm. In some embodiments, the average longest dimension of the particle is from about 350 nm to about 600 nm. In some embodiments, the average longest dimension of the particle is from about 400 nm to about 500 nm. In some embodiments, the average longest dimension of the particle is about 500 nm. In some embodiments, the average longest dimension of the particle is about 400 nm. In some embodiments, the average longest dimension of the particle is about 400 nm, 450 nm, 500 nm, or 550 nm. In some embodiments, the average longest dimension of the particle is about 410 nm, 420 nm, 430 nm, 440 nm or 450 nm. In some embodiments, the average longest dimension of the particle is about 460 nm, 470 nm, 480 nm, 490 nm or 500 nm. In embodiments, the average longest dimension of the particle is at least, about, or at most 100, 200, 300, 400, 500, 600, 700, 800, 900, 1000 nm or a number or a range between any two of these values.

In embodiments, the tissue section is attached to the polymer. In embodiments, the tissue section is attached to the polymer (e.g., as described herein). In embodiments, the thickness (i.e., height or depth) of the polymer is about 1 nm-200 nm. In embodiments, the thickness (i.e., height or depth) of the polymer is about 5 nm. In embodiments, the thickness (i.e., height or depth) of the hydrogel is polymer about 10 nm. In embodiments, the thickness (i.e., height or depth) of the polymer is about 15 nm. In embodiments, the thickness (i.e., height or depth) of the polymer is about 20 nm. In embodiments, the thickness (i.e., height or depth) of the polymer is about 1 nm, 5 nm, 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, or 200 nm. The thickness of the polymer (i.e., height or depth) of the polymer may be controlled by modulating the identities and/or ratios of the copolymers and the crosslinking time.

In embodiments, the polymer includes N-Hydroxyethyl acrylamide (HEAA). In embodiments, the polymer includes 2-Hydroxyethyl Acrylate (HEA). In embodiments, the polymer includes 2-Hydroxyethyl methacrylate (HEMA). In embodiments, the polymer includes Acrylic acid (AA). In embodiments, the polymer includes N-vinyl pyrrolidone (NVP). In embodiments, the polymer includes N-isopropylacrylamide (NIPAM). In embodiments, the polymer includes acrylamide. In embodiments, the polymer includes poly(ethylene glycol) methacrylate (PEGMA). In embodiments, the polymer includes Glycidyl methacrylate (GMA). In embodiments, the polymer includes Dopamine methacrylate (DMA). In embodiments, the polymer includes Acrylic acid N-hydroxysuccinimide ester (AA-NHS). In embodiments, the polymer includes 2-Isocyanatoethyl Acrylate (ICEA). In embodiments, the polymer includes N-(3-aminopropyl)methacrylamide hydrochloride (APMA). In embodiments, the polymer includes 2-hydroxyethyl methacrylate (HEMA) modified with N-hydroxysuccinimide (HEMA-NHS). In embodiments, the polymer includes Epoxypropyl Methacrylate (EPMA). In embodiments, the polymer includes Glycidyl Acrylate (GA). In embodiments, the polymer includes Glycidyl Ethacrylate (GEA). In embodiments, the polymer includes and 3,4-Epoxybutyl Methacrylate (EBMA). In embodiments, the polymer includes [2-(Acryloyloxy)ethyl]trimethylammonium chloride (AETA). In embodiments, the polymer includes methacryloxypropyl trimethyl ammonium chloride (MPTA). In embodiments, the polymer includes dimethylaminoethyl methacrylate (DMAEMA). In embodiments, the polymer includes trimethylammonium ethyl methacrylate chloride (TMAEMC). In embodiments, the polymer includes methacryloxyethyltrimethyl ammonium chloride (METMAC). In embodiments, the polymer includes allyltrimethyl ammonium chloride (ATMAC). In embodiments, the polymer includes vinylbenzyl trimethyl ammonium chloride (VBTMAC). In embodiments, the polymer includes (3-Acrylamidopropyl)trimethylammonium chloride (AATA). In embodiments, the polymer includes [2-(Methacryloyloxy)ethyl]trimethylammonium chloride (META).

In embodiments, the polymer includes N-Hydroxyethyl acrylamide (HEAA), Glycidyl methacrylate (GMA), and [2-(Acryloyloxy)ethyl]trimethylammonium chloride (AETA). In embodiments, the polymer includes acrylic acid (AA) and glycidyl methacrylate (GMA).

In embodiments, the polymer includes methacrylic acid NHS, methacrylic acid, and polyethylene glycol dimethacrylate. In embodiments, the polymer includes Glycidyl methacrylate, methacrylic acid, and polyethylene glycol dimethacrylate. In embodiments, the polymer includes dopamine methacrylate, methacrylic acid, and polyethylene glycol dimethacrylate. In embodiments, the polymer includes N-Hydroxyethyl acrylamide (HEAA), Glycidyl methacrylate (GMA), and polyethylene glycol dimethacrylate. In embodiments, the polymer includes N-Hydroxyethyl acrylamide (HEAA), Glycidyl methacrylate (GMA), methacrylic acid, and polyethylene glycol dimethacrylate. In embodiments, the polymer includes N-Hydroxyethyl acrylamide (HEAA), Glycidyl methacrylate (GMA), zwitterionic sulfobetaine methacrylate (SBMA), and polyethylene glycol dimethacrylate.

In embodiments, the polymer further includes a crosslinker. In embodiments, the crosslinker includes diallyltartramide, divinylbenzene (DVB), allyl methacrylate (AMA), triallyl cyanurate (TAC), N,N′-methylenebisacrylamide (MBAA), ethylene glycol dimethacrylate (EGDMA), diallyl phthalate (DAP), polyethylene glycol diacrylate (PEGDA), genipin, tetraethylene glycol dimethacrylate (TEGDMA), chitosan and genipin, or polyvinyl alcohol (PVA) and borate ions. In embodiments, the crosslinker includes diallyltartramide.

In an aspect is provided a flow cell assembly. In embodiments, the flow cell assembly includes a first solid support; a polymer attached to the first solid support; a cell or tissue attached to the polymer; a second solid support attached to the first solid support, wherein the second solid support is configured to define a reaction chamber. In embodiments, the second solid support is configured to define a reaction chamber when attached to the first solid support. In embodiments, the flow cell assembly includes a frame configured to retain the flow cell assembly. Suitable flow cell frames and handles are described in U.S. Pat. No. 11,747,262. The frame can be configured to retain the flow cell such that a maximal surface area of the flow cell can be available to be exposed to an optical lens (e.g., the optical lens of a nucleic acid sequencing device). The optical lens (e.g., the optical lens of the sequencing device) can be configured to detect excitation, emission, or other signals present on the flow cell. The frame can be configured to retain the flow cell such that a maximal surface area of the flow cell can be available to be in contact with the receiver of a nucleic acid sequencer. The retaining of the flow cell further can include constraining a first, a second, a third, a fourth, a fifth, and a sixth degree of freedom of the flow cell. The frame can be an injection molded frame. The handle can be a raised handle. The frame can be further configured to provide a gap between a work surface and the flow cell. The frame further can include at least one ferromagnetic pin. The at least one biasing feature can be a spring finger. The at least one biasing feature can be a tab. The flow cell can further include a microchip. The microchip can be an electronically erasable programmable read only memory (EEPROM) chip.

The solid supports for some embodiments have at least one surface located within a flow cell. Flow cells provide a convenient format for housing an array of clusters produced by the methods described herein, in particular when subjected to an SBS or other detection technique that involves repeated delivery of reagents in cycles. In embodiments, the polymer is indirectly attached to the solid support by way of being in direct contact with one or more intermediate layers between the solid support and the polymer. In embodiments, one or more intermediate layers between the solid support and the polymer includes an infrared (IR) reflective coating (e.g., an infrared reflective coating described herein). In embodiments, the polymer is directly attached to the solid support. In embodiments, the polymer is a polymer as described herein. In embodiments, the copolymer includes a first monomer, second monomer, and third monomer, wherein the first monomer, second monomer, and third monomer are different.

In embodiments, the solid support further includes a resist (e.g., a resist polymer), wherein the resist is between the copolymer and the solid support. In embodiments, the resist is attached to the copolymer.

In embodiments, the resist is a polydimethylsiloxane (PDMS), poly(methyl methacrylate) (PMMA), cyclic olefin copolymer (COC), silsesquioxane resist, an epoxy-based polymer resist, poly(vinylpyrrolidone-vinyl acrylic acid) copolymer resist, an Off-stoichiometry thiol-enes (OSTE) resist, amorphous fluoropolymer resist, a crystalline fluoropolymer resist, polysiloxane resist, or an organically modified ceramic polymer resist.

In embodiments, the first solid support includes a glass substrate. In embodiments, the second solid support includes a glass substrate. In embodiments, the glass substrate is a borosilicate glass substrate with a composition including SiO₂, Al₂O₃, B₂O₃, Li₂O, Na₂O, K₂O, MgO, CaO, SrO, BaO, ZnO, TiO₂, ZrO₂, P₂O₅, or a combination thereof (see e.g., U.S. Pat. No. 10,974,990). In embodiments, the glass substrate is an alkaline earth boro-aluminosilicate glass substrate.

In embodiments, the first solid support includes one or more channel(s). In embodiments, the first solid support includes a channel bored into the first solid support. In embodiments, the first solid support includes a plurality of channels bored into the first solid support. In embodiments, the first solid support includes 2 channels bored into the first solid support. In embodiments, the first solid support includes 3 channels bored into the first solid support. In embodiments, the first solid support includes 4 channels bored into the first solid support. In embodiments, the width of the channel is from about 1 to 5 mm. In embodiments, the width of the channel is from about 5 to about 10 mm. In embodiments, the width of the channel is from about 10 to about 15 mm. In embodiments, the width of the channel is about 5 mm. In embodiments, the width of the channel is about 11 mm. In embodiments, the second solid support includes one or more channel(s). In embodiments, the second solid support includes a channel bored into the second solid support. In embodiments, the second solid support includes a plurality of channels bored into the second solid support. In embodiments, the second solid support includes 2 channels bored into the second solid support. In embodiments, the second solid support includes 3 channels bored into the second solid support. In embodiments, the second solid support includes 4 channels bored into the second solid support. In embodiments, the width of the channel is from about 1 to about 5 mm. In embodiments, the width of the channel is from about 5 to about 10 mm. In embodiments, the width of the channel is from about 10 to 15 mm. In embodiments, the width of the channel is about 5 mm. In embodiments, the width of the channel is about 11 mm.

In embodiments, the second solid support includes a gasket (alternatively referred to herein as a spacer), wherein the gasket defines the reaction chamber. In embodiments, the gasket defines a perimeter of a channel. In embodiments, the gasket includes silicone, polyimide, fluorocarbon elastomer, ethylene propylene diene, polychloroprene, polytetrafluoroethylene, nitrile rubber, butyl rubber, natural rubber, thermoplastic elastomer, or a combination thereof. In embodiments, the second solid support includes a spacer element to form an offset surface. In embodiments, the second solid support includes one or more channels. The channel(s) may be formed by affixing a spacer element to create a defined gap or channel through which liquid can flow or be contained. The spacer element may be made of any suitable material, for example resin, glass, plastic, silicon, an adhesive, or a combination thereof. In embodiments, the spacer element includes a first adhesive in contact with the functionalized glass slide and second adhesive in contact with the second solid support. In embodiments, the spacer element includes a first adhesive in contact with the functionalized glass slide, a second adhesive in contact with the second solid support, and a carrier material in contact with the first adhesive and the second adhesive. The depth of the resulting channel may be controlled by including a carrier material (e.g., one or more polymer or copolymer layers) between the adhesives. In embodiments, the spacer element may form the walls of the reaction chamber, wherein the reaction chamber includes the sample. In embodiments, the spacer element is further attached to the copolymer attached the polymer of the first solid support. In embodiments, the gasket is referred to as a spacer element.

In embodiments, the flow cell assembly further includes 2, 3, 4, 5, 6, 7, 8, 9, 10, 11, or 12 reaction chambers (e.g., channels). In embodiments, the flow cell assembly includes 2 distinct reaction chambers (e.g., channels). In embodiments, the flow cell assembly includes 4 distinct reaction chambers (e.g., channels). In embodiments, each reaction chamber includes a depth of about 50 μm to about 150 μm. In embodiments, the reaction chamber includes a depth of about 80 μm to about 110 μm. In embodiments, the reaction chamber includes a width of about 4 μm to about 15 μm.

In embodiments, the reaction chamber is a channel on the flow cell. In embodiments, the channel includes a depth of about 50 μm to about 150 μm. In embodiments, the channel includes a depth of about 50 μm. In embodiments, the channel includes a depth of about 60 μm. In embodiments, the channel includes a depth of about 70 μm. In embodiments, the channel includes a depth of about 80 μm. In embodiments, the channel includes a depth of about 90 μm. In embodiments, the channel includes a depth of about 100 μm. In embodiments, the channel includes a depth of about 110 μm. In embodiments, the channel includes a depth of about 120 μm. In embodiments, the channel includes a depth of about 130 μm. In embodiments, the channel includes a depth of about 140 μm. In embodiments, the channel includes a depth of about 71 μm. In embodiments, the channel includes a depth of about 72 μm. In embodiments, the channel includes a depth of about 73 μm. In embodiments, the channel includes a depth of about 74 μm. In embodiments, the channel includes a depth of about 75 μm. In embodiments, the channel includes a depth of about 76 μm. In embodiments, the channel includes a depth of about 77 μm. In embodiments, the channel includes a depth of about 78 μm. In embodiments, the channel includes a depth of about 79 μm. In embodiments, the channel includes a depth of 50 μm to 150 μm. The depth of the channel may be referred to as the height of the channel or the distance between the first and second solid supports. In embodiments, the channel includes a depth of 50 μm. In embodiments, the channel includes a depth of 60 μm. In embodiments, the channel includes a depth of 70 μm. In embodiments, the channel includes a depth of 80 μm. In embodiments, the channel includes a depth of 90 μm. In embodiments, the channel includes a depth of 100 μm. In embodiments, the channel includes a depth of 110 μm. In embodiments, the channel includes a depth of 120 μm. In embodiments, the channel includes a depth of 130 μm. In embodiments, the channel includes a depth of 140 μm. In embodiments, the channel includes a depth of 150 μm. In embodiments, the channel includes a depth of 160 μm. In embodiments, the channel includes a depth of 170 μm. In embodiments, the channel includes a depth of 180 μm. In embodiments, the channel includes a depth of 190 μm. In embodiments, the channel includes a depth of 200 μm.

In embodiments, the first solid support or the second solid support includes a port. In embodiments, the first solid support or the second solid support includes an inlet port and an outlet port. In embodiments, the first solid support includes an inlet port and an outlet port. In embodiments, the second solid support includes an inlet port and an outlet port. In embodiments, the first solid support includes an inlet port. In embodiments, the second solid support includes an inlet port. In embodiments, the first solid support includes an outlet port. In embodiments, the second solid support includes an outlet port. In embodiments, each port is about 50 to about 100 mm in diameter. In embodiments, each port is 50 to 100 mm in diameter. In embodiments, each port is 50, 55, 60, 65, 70, 75, 80, 85, 90, 95, or 100 mm in diameter. In embodiments, each port is 70, 75, or 80 mm in diameter.

In embodiments, the first solid support includes a pressure sensitive adhesive (PSA) attached to a glass slide, wherein the glass slide includes inlet and outlet ports. In embodiments, first solid support includes a pressure sensitive adhesive (PSA) laminated to a glass slide, wherein the glass slide includes inlet and outlet ports. In embodiments, the second solid support includes a pressure sensitive adhesive (PSA) attached to a glass slide, wherein the glass slide includes inlet and outlet ports. In embodiments, second solid support includes a pressure sensitive adhesive (PSA) laminated to a glass slide, wherein the glass slide includes inlet and outlet ports. In embodiments, the pressure sensitive adhesive has a thickness of about 10 μm to about 100 μm. In embodiments, the pressure sensitive adhesive has a thickness of about 70 μm to about 100 μm. In embodiments, the pressure sensitive adhesive has a thickness of about 100 μm to about 200 μm. In embodiments, the pressure sensitive adhesive has a thickness of about 200 μm to about 500 μm. In embodiments, the pressure sensitive adhesive has a thickness of about 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, 30 μm, 31 μm, 32 μm, 33 μm, 34 μm, 35 μm, 36 μm, 37 μm, 38 μm, 39 μm, 40 μm, 41 μm, 42 μm, 43 μm, 44 μm, 45 μm, 46 μm, 47 μm, 48 μm, 49 μm, 50 μm, 51 μm, 52 μm, 53 μm, 54 μm, 55 μm, 56 μm, 57 μm, 58 μm, 59 μm, 60 μm, 61 μm, 62 μm, 63 μm, 64 μm, 65 μm, 66 μm, 67 μm, 68 μm, 69 μm, 70 μm, 71 μm, 72 μm, 73 μm, 74 μm, 75 μm, 76 μm, 77 μm, 78 μm, 79 μm, 80 μm, 81 μm, 82 μm, 83 μm, 84 μm, 85 μm, 86 μm, 87 μm, 88 μm, 89 μm, 90 μm, 91 μm, 92 μm, 93 μm, 94 μm, 95 μm, 96 μm, 97 μm, 98 μm, 99 μm, 100 μm, 101 μm, 102 μm, 103 μm, 104 μm, 105 μm, 106 μm, 107 μm, 108 μm, 109 μm, 110 μm, 111 μm, 112 μm, 113 μm, 114 μm, 115 μm, 116 μm, 117 μm, 118 μm, 119 μm, 120 μm, 121 μm, 122 μm, 123 μm, 124 μm, 125 μm, 126 μm, 127 μm, 128 μm, 129 μm, 130 μm, 131 μm, 132 μm, 133 μm, 134 μm, 135 μm, 136 μm, 137 μm, 138 μm, 139 μm, 140 μm, 141 μm, 142 μm, 143 μm, 144 μm, 145 μm, 146 μm, 147 μm, 148 μm, 149 μm, 150 μm or greater. In embodiments, the pressure sensitive adhesive has a thickness of about 50 μm, 55 μm, 60 μm, 65 μm, 70 μm, 75 μm, 80 μm, 85 μm, 90 μm, 95 μm, 100 μm, 105 μm, 110 μm, 115 μm, 120 μm, 125 μm, 130 μm, 135 μm, 140 μm, 145 μm, 150 μm, 155 μm, 160 μm, 165 μm, 170 μm, 175 μm, 180 μm, 185 μm, 190 μm, 195 μm, 200 μm, 205 μm, 210 μm, 215 μm, 220 μm, 225 μm, 230 μm, 235 μm, 240 μm, 245 μm, 250 μm, or greater.

In embodiments, the pressure sensitive adhesive (PSA) includes to a first adhesive attached to a carrier polymer, where the carrier polymer is further attached to a second adhesive. In embodiments, the carrier polymer is between the first adhesive and the second adhesive. In embodiments, the adhesive includes an acrylic material. In embodiments, the adhesive includes rubber. In embodiments, the adhesive includes silicone. In embodiments, a variety of adhesive materials are utilized for fabricating leak-free chambers in a flow cell, each selected based on their unique properties and the specific requirements of the application. Acrylic-based adhesives are favored for their strong bond and resistance to environmental factors, while rubber-based adhesives are chosen for their flexibility and resilience in applications requiring movement. Silicone adhesives are notable for their high-temperature resistance and moisture-proof sealing capabilities. Epoxy resins offer unparalleled strength and chemical resistance, making them ideal for demanding industrial applications. Polyurethane adhesives, known for their balance of strength, flexibility, and chemical resistance, are versatile in bonding diverse materials. Lastly, cyanoacrylates, are valued for their rapid setting and strong bonding properties, essential for quick and reliable leak prevention.

In embodiments, the carrier structure includes polyimide, polyester (PET), polypropylene (PP), polyvinyl chloride (PVC), paper, acrylic foam, polyethylene foam, urethane foam, polyvinyl chloride form, glass, or a combination thereof. In embodiments, the carrier structure is a carrier polymer. In embodiments, the carrier polymer of the PSA includes polyester (PET). In embodiments, the carrier polymer of the PSA includes polyimide. In embodiments, the carrier polymer is chemically inert. In embodiments, the carrier polymer does not react with reagents used for sample preparation. In embodiments, the carrier polymer does not react with reagents used for amplification. In embodiments, the carrier polymer does not react with reagents used for sequencing. In embodiments, the carrier polymer does not react with reagents used for imaging. In embodiments, the carrier polymer does not react with reagents used for detection of the biomolecule of the tissue section or cell as described herein. In embodiments, the first adhesive and second adhesive both include silicone and the carrier polymer includes polyimide. In embodiments, the first adhesive and second adhesive includes silicone and the carrier polymer includes polyester.

In embodiments, the adhesive has a thickness of about 10 μm to about 100 μm. In embodiments, the adhesive has a thickness of about 10 μm to about 30 μm. In embodiments, the adhesive has a thickness of about 30 μm to about 60 μm. In embodiments, the adhesive has a thickness of about 70 μm to about 100 μm. In embodiments, the adhesive has a thickness of about 100 μm to about 200 μm. In embodiments, the adhesive has a thickness of about 10 μm.

In embodiments, the spacer element (e.g., the combination of the first adhesive, carrier polymer, and second adhesive) has a total thickness of about 50 μm to about 150 μm. In embodiments, the spacer element including the first adhesive, carrier polymer, and second adhesive has a total thickness of about 50 μm to about 60 μm. In embodiments, the spacer element including the first adhesive, carrier polymer, and second adhesive has a total thickness of about 60 μm to about 70 μm. In embodiments, the spacer element including the first adhesive, carrier polymer, and second adhesive has a total thickness of about 70 μm to about 80 μm. In embodiments, the spacer element including the first adhesive, carrier polymer, and second adhesive has a total thickness of about 80 μm to about 90 μm. In embodiments, the spacer element including the first adhesive, carrier polymer, and second adhesive has a total thickness of about 90 μm to about 100 μm. In embodiments, the spacer element including the first adhesive, carrier polymer, and second adhesive has a total thickness of about 100 μm to about 110 μm. In embodiments, the spacer element including the first adhesive layer, carrier polymer, and second adhesive has a total thickness of about 110 μm to about 120 μm. In embodiments, the spacer element including the first adhesive, carrier polymer, and second adhesive has a total thickness of about 120 μm to about 130 μm. In embodiments, the spacer element including the first adhesive, carrier polymer, and second adhesive has a total thickness of about 130 μm to about 140 μm. In embodiments, the spacer element including the first adhesive, carrier polymer, and second adhesive has a total thickness of about 140 μm to about 150 μm.

In embodiments, the spacer element is a pressure sensitive adhesive (PSA). In embodiments, the PSA is attached to the first solid support and second solid support, and the distance between the first solid support and second solid support is between about 50 μm to about 120 μm. In embodiments, the distance between the first solid support and second solid support is between about 60 μm to about 70 μm. In embodiments, the distance between the first solid support and second solid support is about 70 μm to about 80 μm. In embodiments, the distance between the first solid support and second solid support is about 80 μm to about 90 μm. In embodiments, the distance between the first solid support and second solid support is about 90 μm to about 100 μm. In embodiments, the spacer element is a pressure sensitive adhesive (PSA). In embodiments, the PSA is attached to the first solid support and second solid support, and the distance between the first solid support and second solid support is about 50 μm, about 60 μm, about 70 μm, about 80 μm, about 90 μm, about 100 μm, about 110 μm, about 120 μm, about 130 μm, about 140 μm, about 150 μm, about 160 μm, about 170 μm, about 180 μm, about 190 μm, about 200 μm, about 210 μm, about 220 μm, about 230 μm, about 240 μm, about 250 μm, about 260 μm, about 270 μm, about 280 μm, about 290 μm, about 300 μm, about 310 μm, about 320 μm, about 330 μm, about 340 μm, about 350 μm, about 360 μm, about 370 μm, about 380 μm, about 390 μm, about 400 μm, about 410 μm, about 420 μm, about 430 μm, about 440 μm, about 450 μm, about 460 μm, about 470 μm, about 480 μm, about 490 μm, or about 500 μm.

In embodiments, the first solid support includes a plurality of channels etched in glass that is capable of being in contact with a UV-curable adhesive. In embodiments, the second solid support includes a plurality of channels etched in glass that is capable of being in contact with a UV-curable adhesive. A UV-curable adhesive is an adhesive that hardens or sets when exposed to ultraviolet light. In embodiments, the UV-curing adhesive cures when exposed to wavelengths between about 365 nm to about 405 nm. In embodiments, the UV-curing adhesive cures when exposed to wavelength of about 405 nm. In embodiments, the UV-curable adhesive is chemically compatible with glass. The UV-curing adhesive includes a mixture of photo-initiator that, upon exposure to UV light, initiates a polymerization reaction that converts the liquid adhesive into a solid polymer, resulting in a rapid curing process. In embodiments, use of a UV-curing adhesive on the first solid support provides channel depth consistency and a leak-free seal. In embodiments, use of a UV-curing adhesive on the second solid support provides channel depth consistency and a leak-free seal.

In an aspect is provided a microfluidic device including the flow cell assembly as described herein. In embodiments, the microfluidic device includes two or four flow cells. In embodiments, the microfluidic device includes a functionalized tissue slide as described herein. In embodiments, the microfluidic device includes an imaging system or detection apparatus. Any of a variety of detection apparatus can be configured to detect the reaction vessel or solid support where reagents interact. Examples include luminescence detectors, surface plasmon resonance detectors and others known in the art. Exemplary systems having fluidic and detection components that can be readily modified for use in a system herein include, but are not limited to, those set forth in U.S. Pat. Nos. 8,241,573, 8,039,817; or US Pat. App. Pub. No. 2012/0270305 A1, each of which is incorporated herein by reference. In embodiments, the microfluidic device further includes one or more excitation lasers.

In embodiments, the microfluidic device is a nucleic acid sequencing device including: a stage configured to hold an array or solid support as described herein, including embodiments; an array or solid support as described herein, including embodiments; and a detector for obtaining sequencing data. In some embodiments, the detector is an imaging detector, such as a CCD, EMCCD, or s-CMOS detector. Nucleic acid sequencing devices utilize excitation beams to excite labeled nucleotides in the DNA containing sample to enable analysis of the base pairs present within the DNA. Many of the next-generation sequencing (NGS) technologies use a form of sequencing by synthesis (SBS), wherein modified nucleotides are used along with an enzyme to read the sequence of DNA templates in a controlled manner. In embodiments, sequencing includes a sequencing by synthesis event, where individual nucleotides are identified iteratively (e.g., incorporated and detected into a growing complementary strand), as they are polymerized to form a growing complementary strand. In embodiments, nucleotides added to a growing complementary strand include both a label and a reversible chain terminator that prevents further extension, such that the nucleotide may be identified by the label before removing the terminator to add and identify a further nucleotide. Such reversible chain terminators include removable 3′ blocking groups, for example as described in U.S. Pat. Nos. 10,738,072, 7,541,444 and 7,057,026. Once such a modified nucleotide has been incorporated into the growing polynucleotide chain complementary to the region of the template being sequenced, there is no free 3′-OH group available to direct further sequence extension and therefore the polymerase cannot add further nucleotides. Once the identity of the base incorporated into the growing chain has been determined, the 3′ reversible terminator may be removed to allow addition of the next successive nucleotide. In embodiments, the nucleic acid sequencing device utilizes the detection of four different nucleotides that include four different labels.

The term “nucleic acid sequencing device” means an integrated system of one or more chambers, ports, and channels that are interconnected and in fluid communication and designed for carrying out an analytical reaction or process, either alone or in cooperation with an appliance or instrument that provides support functions, such as sample introduction, fluid and/or reagent driving means, temperature control, detection systems, data collection and/or integration systems, for the purpose of determining the nucleic acid sequence of a template polynucleotide. Nucleic acid sequencing devices may further include valves, pumps, and specialized functional coatings on interior walls. Nucleic acid sequencing devices may include a receiving unit, or platen, that orients the flow cell such that a maximal surface area of the flow cell is available to be exposed to an optical lens. Other nucleic acid sequencing devices include those provided by Singular Genomics™ such as the G4X™ sequencing platform. Nucleic acid sequencing devices may further include fluidic reservoirs (e.g., bottles), valves, pressure sources, pumps, sensors, control systems, valves, pumps, and specialized functional coatings on interior walls. In embodiments, the device includes a plurality of a sequencing reagent reservoirs and a plurality of clustering reagent reservoirs. In embodiments, the clustering reagent reservoir includes amplification reagents (e.g., an aqueous buffer containing enzymes, salts, and nucleotides, denaturants, crowding agents, etc.) In embodiments, the reservoirs include sequencing reagents (such as an aqueous buffer containing enzymes, salts, and nucleotides); a wash solution (an aqueous buffer); a cleave solution (an aqueous buffer containing a cleaving agent, such as a reducing agent); or a cleaning solution (a dilute bleach solution, dilute NaOH solution, dilute HCl solution, dilute antibacterial solution, or water). The fluid of each of the reservoirs can vary. The fluid can be, for example, an aqueous solution which may contain buffers (e.g., saline-sodium citrate (SSC), ascorbic acid, tris(hydroxymethyl)aminomethane or “Tris”), aqueous salts (e.g., KCl or (NH₄)₂SO₄)), nucleotides, polymerases, cleaving agent (e.g., tri-n-butyl-phosphine, triphenyl phosphine and its sulfonated versions (i.e., tris(3-sulfophenyl)-phosphine, TPPTS), and tri(carboxyethyl)phosphine (TCEP) and its salts, cleaving agent scavenger compounds (e.g., 2′-Dithiobisethanamine or 11-Azido-3,6,9-trioxaundecane-1-amine), chelating agents (e.g., EDTA), detergents, surfactants, crowding agents, or stabilizers (e.g., PEG, Tween, BSA). Non-limited examples of reservoirs include cartridges, pouches, vials, containers, and Eppendorf tubes. In embodiments, the device is configured to perform fluorescent imaging. In embodiments, the device includes one or more light sources (e.g., one or more lasers). In embodiments, the illuminator or light source is a radiation source (i.e., an origin or generator of propagated electromagnetic energy) providing incident light to the sample. A radiation source can include an illumination source producing electromagnetic radiation in the ultraviolet (UV) range (about 200 to 390 nm), visible (VIS) range (about 390 to 770 nm), or infrared (IR) range (about 0.77 to 25 microns), or other range of the electromagnetic spectrum. In embodiments, the illuminator or light source is a lamp such as an arc lamp or quartz halogen lamp. In embodiments, the illuminator or light source is a coherent light source. In embodiments, the light source is a laser, LED (light emitting diode), a mercury or tungsten lamp, or a super-continuous diode. In embodiments, the light source provides excitation beams having a wavelength between 200 nm to 1500 nm. In embodiments, the laser provides excitation beams having a wavelength of 405 nm, 470 nm, 488 nm, 514 nm, 520 nm, 532 nm, 561 nm, 633 nm, 639 nm, 640 nm, 800 nm, 808 nm, 912 nm, 1024 nm, or 1500 nm. In embodiments, the illuminator or light source is a light-emitting diode (LED). The LED can be, for example, an Organic Light Emitting Diode (OLED), a Thin Film Electroluminescent Device (TFELD), or a Quantum dot based inorganic organic LED. The LED can include a phosphorescent OLED (PHOLED). In embodiments, the nucleic acid sequencing device includes an imaging system (e.g., an imaging system as described herein). The imaging system capable of exciting one or more of the identifiable labels (e.g., a fluorescent label) linked to a nucleotide and thereafter obtain image data for the identifiable labels. The image data (e.g., detection data) may be analyzed by another component within the device. The imaging system may include a system described herein and may include a fluorescence spectrophotometer including an objective lens and/or a solid-state imaging device. The solid-state imaging device may include a charge coupled device (CCD) and/or a complementary metal oxide semiconductor (CMOS).

The system may also include circuitry and processors, including systems using microcontrollers, reduced instruction set computers (RISC), application specific integrated circuits (ASICs), field programmable gate array (FPGAs), logic circuits, and any other circuit or processor capable of executing functions described herein. The set of instructions may be in the form of a software program. As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. In embodiments, the device includes a thermal control assembly useful to control the temperature of the reagents.

In an aspect is provided a kit, including the solid support or flow cell assembly as described herein. In an aspect is provided a kit, including the first solid support attached to a polymer, which is further attached to an IR reflective coating, coating agent, and/or particles; a copolymer attached to the polymer, and a second solid support, configured to define a reaction chamber when attached to the first solid support. Generally, the kit includes one or more containers providing a composition and one or more additional reagents (e.g., a buffer suitable for polynucleotide extension). The kit may also include a template nucleic acid (DNA and/or RNA), one or more primer polynucleotides, nucleoside triphosphates (including, e.g., deoxyribonucleotides, ribonucleotides, particles, polymerases, labeled nucleotides, and/or modified nucleotides), buffers, salts, and/or labels (e.g., fluorophores). In embodiments, the kit includes a plurality of detection agents capable of detecting a biomolecule (or plurality thereof) from a tissue section. In embodiments, the kit includes the tissue section including the biomolecule to be detected (or plurality thereof) already immobilized onto the first solid support of the flow cell assembly as described herein. In embodiments, kit includes the flow cell assembly as described herein and a flow cell carrier (e.g., a flow cell carrier as described in U.S. Pat. No. 11,747,262, which is incorporated herein by reference for all purposes).

In embodiments, the kit includes a first solid support (e.g., a first solid support as described herein), wherein a polymer attached to the first solid support; and a copolymer attached to the first polymer. In embodiments, the first solid support is not attached to the second solid support until the tissue sample is affixed to the first solid support. The first solid support is configured to remain detached from the second solid support until the user affixes the tissue sample to the copolymer, ensuring precise placement and alignment of the biological material. In embodiments, the kit includes a second solid support, wherein the second support is configured to define a reaction chamber. In embodiments, the kit includes a second solid support comprising a glass slide with one or more (e.g., eight) pre-drilled ports enabling the introduction and removal of reagents. In embodiments, the second solid support is further equipped with a gasket that is pre-attached to the glass slide. The gasket, having a peel-off backing, is designed to form a sealed reaction chamber when adhered to the first solid support. This design ensures the creation of defined channels necessary for fluid flow and biochemical reactions within the assembled flow cell. Upon receiving the kit, users are instructed to affix one or more tissue sample(s) onto the first solid support, directly onto the exposed copolymer. After the sample deposition, the user will remove the peel-off backing from the gasket on the second solid support and align it with the first solid support. This alignment and subsequent attachment create a secure and sealed environment, forming the reaction chamber with integrated microfluidic channels as dictated by the configuration of the gasket. The flow cell assembly process is designed to be straightforward, allowing for efficient setup while minimizing the potential for user error and ensuring reproducibility of experimental conditions. In embodiments, the second support includes 1, 2, or 4 distinct reaction chambers. In embodiments, the second solid support includes a gasket, wherein the gasket defines the reaction chamber. In embodiments, the gasket includes silicone, polyimide, fluorocarbon elastomer, ethylene propylene diene, polychloroprene, polytetrafluoroethylene, nitrile rubber, butyl rubber, natural rubber, thermoplastic elastomer, or a combination thereof. In embodiments, the second solid support includes a spacer element to form an offset surface when attached to the first solid support. In embodiments, the second solid support includes one or more channels. The channel(s) may be formed by affixing a spacer element to create a defined gap or channel through which liquid can flow or be contained. The spacer element may be made of any suitable material, for example resin, glass, plastic, silicon, an adhesive, or a combination thereof. In embodiments, the spacer element includes a first adhesive in contact with the functionalized glass slide and second adhesive in contact with the second solid support. In embodiments, the spacer element includes a first adhesive in contact with the first solid support, a second adhesive in contact with the second solid support, and a carrier material in contact with the first adhesive and the second adhesive.

### III. Methods

In an aspect is provided a method of making a solid support, the method including: binding a first polymer to the solid support and contacting the first polymer with a deprotection agent (e.g., a reducing agent) to generate a second polymer. In embodiments, the first polymer includes polymerized subunits as described herein (e.g., Formula I). In embodiments, the second polymer includes polymerized subunits as described herein (e.g., Formula I). In embodiments, the first polymer includes polymerized subunits having the formula:

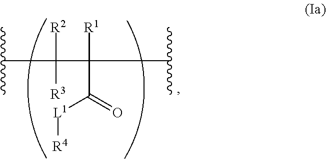

where R¹, R², R³, R⁴, and L¹ are as described herein. In embodiments, the second polymer includes polymerized subunits having the formula:

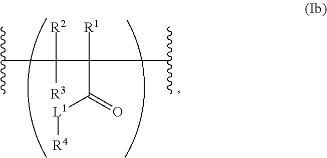

where R¹, R², R³, and L¹ are as described herein. In embodiments, R¹ is hydrogen, substituted or unsubstituted alkyl, or substituted or unsubstituted heteroalkyl. In embodiments, R² is hydrogen or substituted or unsubstituted alkyl. In embodiments, R³ is hydrogen or substituted or unsubstituted alkyl. In embodiments, L¹ is -L^{1A}-L^{1B}-L^{1C}-L^{1D}-L^{1E}-; and L^{1A}, L^{1B}, L^{1C}, L^{1D} and L^{1E} are independently a bond, —NH—, —O—, —S—, substituted or unsubstituted alkylene, or substituted or unsubstituted heteroalkylene. In embodiments, a perimeter of the first solid support has a spacer layer bonded thereto, and after the cell or tissue is applied, the method further includes bonding a second solid support (e.g., a lid) to the spacer layer. In embodiments, the method includes affixing a second solid support to the first solid support, wherein the first solid support or the second solid support includes a port (e.g., an inlet or outlet port). In embodiments, the first solid support includes 2-4 inlet ports and 2-4 outlet ports. In embodiments, the second solid support includes 2-4 inlet ports and 2-4 outlet ports. Fluid channels and chambers may be sealed by placing the first planar surface of the first solid support in contact with, and bonding to, the planar surface of a second solid support to form the channels and/or chambers (e.g., the interior portion) of the device at the interface of these two components. In embodiments, the assembly includes openings that are oriented such that they are in fluid communication with at least one of the fluid channels and/or fluid chambers formed in the interior portion of the device, thereby forming fluid inlets and/or fluid outlets. In some instances, the openings are formed on the first solid support. In embodiments, the openings are formed on the first and the second solid support. In embodiments, the openings are positioned at the top side of the assembly. In embodiments, the openings are positioned at the bottom side of the assembly. In embodiments, the openings are positioned at the first and/or the second ends of the device, and the channels run along the direction from the first end to the second end.

In embodiments, the method further includes contacting the polymer with a plurality of particles. In embodiments, the method further includes sonicating the plurality of particles prior to deposition onto the solid support. In embodiments, the method further includes dissolving the particles in an organic solvent to generate a solution of particles, followed by contacting the polymer or coupling agent with the solution of particles for about 1 minute, about 2 minutes, about 3 minutes, about 4 minutes, about 5 minutes, about 6 minutes, about 7 minutes, about 8 minutes, about 9 minutes, about 10 minutes, about 15 minutes, about 20 minutes, about 30 minutes, about 35 minutes, about 40 minutes, about 45 minutes, about 50 minutes, about 55 minutes, or about 60 minutes.

In embodiments, prior to binding the polymer to the solid support, the method includes rinsing the solid support with an alcohol, where the concentration of alcohol (v/v) is about 90%, about 91%, about 92%, about 93%, about 94%, about 95%, about 96%, about 97%, about 98%, or about 99% alcohol. In embodiments, the alcohol is ethanol, propanol, isopropanol, methanol, or butanol. In embodiments, prior to binding the polymer to the solid support, the method includes rinsing the solid support in ethanol, where the concentration of ethanol (v/v) is about 90%, about 91%, about 92%, about 93%, about 94%, about 95%, about 96%, about 97%, about 98%, or about 99% ethanol. In embodiments, the concentration of ethanol (v/v) is about 95.5% ethanol. In embodiments, the concentration of ethanol (v/v) is about 99.5% ethanol. In embodiments, the concentration of ethanol (v/v) is about 99.9% ethanol.

In embodiments, binding the polymer to the solid support includes contacting (e.g., incubating) the solid support with a solution including the polymer for about 15 minutes, approximately 30 minutes, about 1 hour, about 2 hours, about 3 hours, about 4 hours, about 5 hours, about 6 hours, about 7 hours, about 8 hours, about 9 hours, about 10 hours or more. In embodiments, binding the polymer to the solid support includes contacting the solid support with the polymer for about 30 minutes. In embodiments, binding the polymer to the solid support includes contacting the solid support with the polymer for about 1 hour. In embodiments, binding the polymer to the solid support includes contacting the solid support with the polymer for about 2 hours.

In embodiments, binding the polymer to the solid support further includes rinsing the solid support. In embodiments, rinsing includes contacting with a silane compound (e.g., compounds where silicon may be bonded to groups such as chlorine (as in chlorosilanes), methyl groups (as in alkylsilanes), or a combination of these (as in chlorotrimethylsilane, CTMS). In embodiments, rinsing the solid support includes contacting the solid support with hexamethyldisilazane (HDMS), trimethylchlorosilane (TMCS), tetramethylsilane (TMS), dimethyl sulfoxide (DMSO), trichlorosilane (TMS), cyclohexane, dichlorodimethylsilane, or chlorotrimethylsilane (CTMS).

In embodiments, binding the polymer includes contacting the solid support with the polymer for about 5 minutes, about 10 minutes, about 20 minutes, about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours or more at room temperature.

In an aspect is provided a method of making a flow cell assembly, the method including: binding a polymer to a first solid support; attaching a cell or tissue to the polymer; and affixing a second solid support to the first solid support, wherein the first solid support or the second solid support includes a port (e.g., an inlet or outlet port). In embodiments, the second solid support includes one or more (e.g., 2-4 inlet ports and one or more (e.g., 2-4) outlet ports. In embodiments, the first solid support and second solid support are described herein including embodiments. In embodiments, binding a polymer to a first solid support is as described herein. In embodiments, the second solid support includes an adhesive. In embodiments, the second solid support includes a spacer element. In embodiments, the second solid support is configured to define a reaction chamber when attached to the first solid support. In embodiments, binding a polymer to a first solid support is as described herein.

In embodiments, affixing the second solid support to the first solid support includes applying pressure to create a fluidic leak-free seal between the first and second solid supports. In embodiments, applying pressure forms a bond between the gasket and the first and second solid supports. In embodiments, affixing the second solid support to the first solid support includes using a UV curable adhesive attached to the second solid support, where the UV curable adhesive is cured when exposed to wavelengths between 365 nm to 380 nm. In embodiments, affixing the second solid support to the first solid support includes using a UV curable adhesive attached to the second solid support, where the UV curable adhesive is cured when exposed to wavelengths between 380 nm to 405 nm. In embodiments, affixing the second solid support to the first solid support includes using a UV curable adhesive attached to the second solid support, where the UV curable adhesive is cured when exposed to wavelength of 405 nm.

In embodiments, the UV-curing adhesive cures when exposed to UV light for about 1 minute, about 5 minutes, about 10 minutes, about 15 minutes, about 20 minutes, about 25 minutes, about 30 minutes, about 40 minutes, about 50 minutes, about 60 minutes, about 70 minutes, about 80 minutes, about 90 minutes, or about 100 minutes. In embodiments, the UV-curing adhesive cures when exposed to UV light for about 10 minutes. In embodiments, the UV-curing adhesive cures when exposed to UV light for about 15 minutes. In embodiments, the UV-curing adhesive cures when exposed to UV light for about 20 minutes. In embodiments, the UV-curing adhesive cures when exposed to UV light for about 30 minutes.

In embodiments, affixing the second solid support to the first solid support includes using a spacer element. In embodiments, the spacer element includes an adhesive. In embodiments, the spacer element includes a pressure sensitive adhesive (PSA) attached to the second solid support, where the pressure sensitive adhesive is affixed with the application of pressure of about 5 psi, 6 psi, 7 psi, 8 psi, 9 psi, 10 psi, 11 psi, 12 psi, 13 psi, 14 psi, 15 psi, 16 psi, 17 psi, 18 psi, 19 psi, 20 psi, or more. In embodiments, the spacer element includes a pressure sensitive adhesive (PSA) attached to the second solid support, where the pressure sensitive adhesive is affixed with the application of pressure of about 20 psi, 21 psi, 22 psi, 23 psi, 24 psi, 25 psi, 26 psi, 27 psi, 28 psi, 29 psi, 30 psi, 31 psi, 32 psi, 33 psi, 34 psi, 35 psi, 36 psi, 37 psi, 38 psi, 39 psi, 40 psi, 41 psi, 42 psi, 43 psi, 44 psi, 45 psi, 46 psi, 47 psi, 48 psi, 49 psi, 50 psi, 51 psi, 52 psi, 53 psi, 54 psi, 55 psi, 56 psi, 57 psi, 58 psi, 59 psi, 60 psi, 61 psi, 62 psi, 63 psi, 64 psi, 65 psi, 66 psi, 67 psi, 68 psi, 69 psi, 70 psi, 71 psi, 72 psi, 73 psi, 74 psi, 75 psi, 76 psi, 77 psi, 78 psi, 79 psi, 80 psi, 81 psi, 82 psi, 83 psi, 84 psi, 85 psi, 86 psi, 87 psi, 88 psi, 89 psi, 90 psi, 91 psi, 92 psi, 93 psi, 94 psi, 95 psi, 96 psi, 97 psi, 98 psi, 99 psi, 100 psi, or more. In embodiments, the spacer element includes a pressure sensitive adhesive (PSA) attached to the second solid support, where the pressure sensitive adhesive is affixed with the application of pressure between about 10-15 psi. In embodiments, the spacer element includes a pressure sensitive adhesive (PSA) attached to the second solid support, where the pressure sensitive adhesive is affixed with the application of pressure between about 10-20 psi. In embodiments, the spacer element includes a pressure sensitive adhesive (PSA) attached to the second solid support, where the pressure sensitive adhesive is affixed with the application of pressure of about 10 psi. In embodiments, the spacer element includes a pressure sensitive adhesive (PSA) attached to the second solid support, where the pressure sensitive adhesive is affixed with the application of pressure of about 15 psi. In embodiments, the spacer element includes a pressure sensitive adhesive (PSA) attached to the second solid support, where the pressure sensitive adhesive is affixed with the application of pressure of about 20 psi. In embodiments, the spacer element includes a pressure sensitive adhesive (PSA) attached to the second solid support, where the pressure sensitive adhesive is affixed with the application of pressure of about 25 psi. In embodiments, the spacer element includes a pressure sensitive adhesive (PSA) attached to the second solid support, where the pressure sensitive adhesive is affixed with the application of pressure of about 30 psi. In embodiments, affixing includes heating the assembly (e.g., heating to 40, 50, 60, or 70° C.).

In embodiments, affixing the second solid support to the first solid support placing the first solid support described herein and the second solid support described herein and applying uniform pressure.

In embodiments, the method includes attaching the first solid support and a second solid support with a gasket between the first solid support and the second solid support wherein the gasket is double-sided tape. In embodiments, the method includes attaching the first solid support and a second solid support with a double-sided tape between the first solid support and the second solid support, wherein the first solid support includes drilled ports. In embodiments, the method includes attaching the first solid support and a second solid support with a double-sided tape between the first solid support and the second solid support, wherein the second solid support includes drilled ports.

In embodiments, the thickness of the double-sided tape is 5 about μm, 10 about μm, 15 about μm, 20 about μm, 25 about μm, 30 about μm, 35 about μm, 40 about μm, 45 about μm, or 50 about μm. In embodiments, the thickness of the double-sided tape is 5 about μm. In embodiments, the thickness of the double-sided tape is 10 about μm. In embodiments, the thickness of the double-sided tape is 15 about μm. In embodiments, the thickness of the double-sided tape is 20 about μm.

In an aspect is provided a method of detecting a biomolecule in or on a cell or tissue, the method including immobilizing a cell or tissue including the biomolecule to a first solid support, wherein the first solid support includes a polymer attached to the first solid support as described herein; attaching a second solid support to the first solid support (e.g., to form a channel); contacting the biomolecule in or on the cell or tissue with a detection agent including a label; detecting the label, thereby detecting the biomolecule. In embodiments, the method includes imaging the cell or tissue.

In embodiments, the method includes detecting biomolecules in a tissue, the method including: (i) binding a first polynucleotide probe to a first nucleic acid molecule in the tissue and incorporating a sequence of the first nucleic acid molecule into the first polynucleotide probe; amplifying the first polynucleotide probe to form a first amplification product; and binding a first fluorescently labeled nucleotide to the first amplification product; (ii) binding a second polynucleotide probe to an oligonucleotide, wherein the oligonucleotide is attached to a protein in the tissue; amplifying the second polynucleotide probe to form a second amplification product; and binding a second fluorescently labeled nucleotide to the second amplification product; (iii) contacting the tissue with a stain; and (iv) directing an excitation light to the tissue section and detecting an emission light from the first fluorescently labeled nucleotide, the second fluorescently labeled nucleotide, and the stain.

In embodiments, the method includes immobilizing a cell or tissue including a template polynucleotide to the first solid support, wherein the first solid support includes the copolymer described herein (e.g., the copolymer formulation described herein). In embodiments, the method includes contacting the cell or tissue including the template polynucleotide with an oligonucleotide-specific binding agent including a first target hybridization sequence and a second target hybridization sequence; hybridizing the first target hybridization sequence to the template polynucleotide and hybridizing the second target hybridization sequence to the template polynucleotide; ligating the first target hybridization sequence to the second target hybridization sequence to form a circular polynucleotide; amplifying the circular polynucleotide to form an amplification product; and hybridizing a first sequencing primer to the amplification product, and sequencing the first target hybridization sequence or the second target hybridization sequence.

In embodiments, the method includes immobilizing a cell or tissue including a protein to the first solid support, wherein the first solid support includes the copolymer formulation described herein. In embodiments, the method includes contacting the cell or tissue including the protein with protein-specific binding agent including an oligonucleotide label described herein; ligating the first hybridization sequence to the second hybridization sequence to form a circular polynucleotide; amplifying the circular polynucleotide to form an amplification product; and hybridizing a first sequencing primer to the amplification product, and sequencing the first hybridization sequence or the second hybridization sequence.

In embodiments, the method includes immobilizing a plurality of tissue sections to the first solid support, wherein a tissue in a plurality of tissue sections includes the biomolecule to be detected. In embodiments, the method includes immobilizing 24 tissue sections (10 mm×17 mm sections). In embodiments, the method includes immobilizing 40 tissue sections (10 mm×10 mm sections). In embodiments, the method includes immobilizing 128 tissue sections (4 m×4 m sections).

The cell or tissue may be manipulated prior to immobilizing the cell or tissue onto a solid support using known techniques in the art (see, e.g., PCT Publication WO2023076832A1). In embodiments, the method further includes cutting a sample portion from the biological sample (e.g., including cells or tissues) using a punch device such that the punch device contains the sample portion; mounting the punch device containing the sample portion onto the first solid support as described herein (e.g., inverting the punch device); pushing the sample portion out of the punch device using a piston, so that all or a portion thereof of the sample portion is positioned on the first solid support as described herein. In embodiments, the method further includes cutting a sample portion from the biological sample using two or more punch devices such that each punch device contains a different the sample portion; mounting each punch device containing the sample portion onto the first solid support as described herein; pushing the sample portions out of the punch devices using one or more pistons so that the sample portions are positioned onto the first solid support as described herein.

In embodiments, the biomolecule is a nucleic acid molecule, carbohydrate, or protein. In embodiments, the biomolecule is a nucleic acid molecule. In embodiments, the biomolecule is a carbohydrate. In embodiments, the biomolecule is a protein. The biomolecule(s) to be detected can be any biological molecules including but not limited to proteins, nucleic acids, lipids, carbohydrates, ions, or multicomponent complexes containing any of the above. Examples of subcellular targets include organelles, e.g., mitochondria, Golgi apparatus, endoplasmic reticulum, chloroplasts, endocytic vesicles, exocytic vesicles, vacuoles, lysosomes, etc. Exemplary nucleic acid targets can include genomic DNA of various conformations (e.g., A-DNA, B-DNA, Z-DNA), mitochondria DNA (mtDNA), mRNA, tRNA, rRNA, hRNA, miRNA, and piRNA. In embodiments, the biomolecule is a non-nucleic acid target. Non-nucleic acid targets include, but are not limited to, lipids, carbohydrates, peptides, proteins, glycoproteins, lipoproteins, phosphoproteins, acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In embodiments, the biomolecule is located within the cell. In embodiments, the biomolecule is located on the surface of the cell. In embodiments, the biomolecule is attached on a cell surface, such as a transmembrane analyte.

A biomolecule to be detected or a plurality of biomolecules to be detected using the methods described herein can be isolated, extracted, or obtained from a sample. A sample can be any specimen that is isolated or obtained from a subject or part thereof. A sample can be any specimen that is isolated or obtained from multiple subjects. Non-limiting examples of specimens include fluid or tissue from a subject, including, without limitation, blood or a blood product (e.g., serum, plasma, platelets, buffy coats, or the like), umbilical cord blood, chorionic villi, amniotic fluid, cerebrospinal fluid, spinal fluid, lavage fluid (e.g., lung, gastric, peritoneal, ductal, ear, arthroscopic), a biopsy sample, celocentesis sample, cells (blood cells, lymphocytes, placental cells, stem cells, bone marrow derived cells, embryo or fetal cells) or parts thereof (e.g., mitochondrial, nucleus, extracts, or the like), urine, feces, sputum, saliva, nasal mucous, prostate fluid, lavage, semen, lymphatic fluid, bile, tears, sweat, breast milk, breast fluid, the like or combinations thereof. Non-limiting examples of tissues include organ tissues (e.g., liver, kidney, lung, thymus, adrenals, skin, bladder, reproductive organs, intestine, colon, spleen, brain, the like or parts thereof), epithelial tissue, hair, hair follicles, ducts, canals, bone, eye, nose, mouth, throat, ear, nails, the like, parts thereof or combinations thereof. A sample may include cells or tissues that are normal, healthy, diseased (e.g., infected), and/or cancerous (e.g., cancer cells). A sample obtained from a subject may include cells or cellular material (e.g., nucleic acids) of multiple organisms (e.g., virus nucleic acid, fetal nucleic acid, bacterial nucleic acid, parasite nucleic acid). A sample may include a cell and RNA transcripts. A sample can include nucleic acids obtained from one or more subjects. In some embodiments a sample may include nucleic acid obtained from a single subject.

In embodiments, the detection agent is a biomolecule-specific binding agent. In embodiments, the biomolecule-specific binding agent is a protein-specific binding agent. In embodiments, the biomolecule-specific binding agent is an oligonucleotide-specific binding agent. In embodiments, the biomolecule-specific binding agent is capable of binding to a cluster of differentiation (CD) marker, integrin, selectin, cadherin, cytokine receptor, chemokine receptor, Toll-like receptor (TLR), ion channel, transmembrane protein, lipoprotein, glycoprotein, cell surface protein, transport protein, intracellular organelle, or transcription factor. In embodiments, the intracellular organelle includes actin, carbohydrate, centrosomes and centrioles, chloroplasts (in plant cells and some protists), cytoskeleton, endoplasmic reticulum, endosome, Golgi apparatus, intermediate filaments, lysosome, microfilaments, microtubules, mitochondria, nuclear envelope, nuclear pores, nucleoid, nucleolus, nucleus, peroxisome, phosphatidylserine, plasma membrane, ribosomes, rough endoplasmic reticulum, smooth endoplasmic reticulum, transferrin receptor, transport vesicles, and/or vacuoles. In embodiments, the biomolecule specific binding agent is capable of binding to a biomolecule in the mitogen-activated protein kinase (MAPK) pathway, PI3K/AKT/mTOR pathway, Wnt/β-catenin pathway, intrinsic (mitochondrial) pathway, extrinsic (death receptor) pathway, caspase cascade, Notch signaling pathway, hedgehog signaling pathway, TGF-β (transforming growth factor Beta) pathway, JAK/STAT pathway, G-protein coupled receptor (GPCR) pathway, calcium signaling pathway, glycolysis, citric acid cycle (Krebs Cycle), oxidative phosphorylation, lipid metabolism pathway, amino acid metabolism, Toll-like receptor (TLR) pathway, NF-κB signaling pathway, complement pathway, nucleotide excision repair (NER), base excision repair (BER), mismatch repair (MMR), cyclin-dependent kinase (CDK) pathway, Rb (retinoblastoma) pathway, p53 pathway, unfolded protein response (UPR), heat shock response pathway, oxidative stress pathway, BMP (bone morphogenetic protein) pathway, FGF (fibroblast growth factor) pathway, Sonic Hedgehog pathway, neurotrophin signaling pathway, synaptic transmission pathway, axon guidance pathways, insulin signaling pathway, thyroid hormone pathway, steroid hormone pathway, VEGF (vascular endothelial growth factor) pathway, DNA methylation pathway, histone modification pathway, or angiogenesis. In embodiments, the biomolecule specific binding agent is capable of binding to a biomolecule on the surface of or in a B cell, Mature B Cell, Follicular B cell, Marginal Zone B cell, Short lived plasma cell, Memory B cell, Long lived plasma cell, B1 cell, Breg, Germinal Center B cell, Macrophage, Monocyte, M1 macrophage, M2 macrophage, Dendritic Cell, Plasmacytoid dendritic cell, Monocyte-derived dendritic cell, T cell, T Follicular Helper, Th1, Th2, Th9, Th17, Th22, Treg, platelet (activated), platelet (rested), natural killer cell, neutrophil, basophil, eosinophil, mast cell, astrocyte, neuron, glial cell, lymphocyte, myeloid cell, granulocytes, neural cells, stem cells, endothelial cells, epithelial cells, mesenchymal stem cell, hematopoietic stem cell, embryonic stem, stromal cell, erythrocyte, fibroblast, or apoptotic cell.

In embodiments, the biomolecule is on the surface of the tissue section or on the surface of the cell. In embodiments, the detection agent includes a protein-specific binding agent. In embodiments, the detection agent includes a protein-specific binding agent bound to a nucleic acid sequence (e.g., a nucleic acid label), bioconjugate reactive moiety, an enzyme, or a fluorophore. In embodiments, the protein-specific binding agent is an antibody, single domain antibody, single-chain Fv fragment (scFv), antibody fragment-antigen binding (Fab), affimer, or an aptamer. In embodiments, the protein-specific binding agent is an antibody. In embodiments, the protein-specific binding agent is a single domain antibody. In embodiments, the protein-specific binding agent is a single-chain Fv fragment (scFv). In embodiments, the protein-specific binding agent is an antibody fragment-antigen binding (Fab). In embodiments, the protein-specific binding agent is an affimer. In embodiments, the protein-specific binding agent is an aptamer. In embodiments, a protein-specific binding agent is a protein-specific antibody-oligo (Ab-O) conjugate.

In embodiments, the detection agent includes a protein-specific binding agent or oligonucleotide-specific binding agent. In embodiments, the detection agent includes a protein-specific binding agent. In embodiments, the detection agent includes an oligonucleotide-specific binding agent. In embodiments, the detection agent includes an oligonucleotide-specific binding agent including a nucleic acid sequence, bioconjugate reactive moiety, an enzyme, or a fluorophore.

In embodiments, the detection agent is an oligonucleotide-specific binding agent capable of hybridizing to a target oligonucleotide sequence in a tissue section. In embodiments, the detection agent is an oligonucleotide. In embodiments, the detection agent is an oligonucleotide, wherein the oligonucleotide includes: a) a first region at a 3′ end that is hybridized to a first complementary region of the polynucleotide, and b) a second region at a 5′ end that is hybridized to a second complementary region of the polynucleotide, wherein the second complementary region is 5′ with respect to the first complementary region. In embodiments, the method includes i) circularizing the oligonucleotide agent to generate a circular oligonucleotide and ligating the complementary sequence to the 5′ end of the oligonucleotide-specific binding agent; ii) amplifying the circular oligonucleotide by extending an amplification primer hybridized to the circular oligonucleotide with a strand-displacing polymerase, wherein the amplification primer extension generates an extension product including multiple complements of the circular oligonucleotide; and iii) sequencing the extension product of step (ii). In embodiments, the circular oligonucleotide includes a barcode sequence. In embodiments, circularizing in step i) further includes extending the 3′ end of the oligonucleotide primer (e.g., extending the 3′ end of the primer using a polymerase (e.g., a Thermus thermophilus (Tth) DNA polymerase) to incorporate one or more nucleotides) along the target nucleic acid to generate a complementary sequence (e.g., complementary to the target nucleic acid, for example a target RNA sequence) prior to ligating the complementary sequence to the 5′ end of the oligonucleotide primer.

In embodiments, the label is a nucleic acid sequence. In embodiments, a label is a bioconjugate reactive moiety. In embodiments, a label is an enzyme. In embodiments, a label is a fluorophore. In embodiments, the detection agent includes a label. In embodiments, the label is an oligonucleotide label, wherein the nucleotide sequence of the oligonucleotide label is known a priori (e.g., prior to contacting the detection agent with the biomolecule of interest), and the detection of the oligonucleotide label is associated with the detection of the biomolecule of interest (e.g., a protein or nucleic acid molecule of interest).

In embodiments, the oligonucleotide is about 50 to about 500 nucleotides in length. In embodiments, the oligonucleotide is about 50 to about 300 nucleotides in length. In embodiments, the oligonucleotide is about 80 to about 300 nucleotides in length. In embodiments, the oligonucleotide is about 50 to about 150 nucleotides in length. In embodiments, the oligonucleotide is about or more than about 40, 50, 75, 100, 125, 150, 175, 200, 250, 300, 350, 400, or 500 nucleotides in length. In embodiments, the oligonucleotide is less than about 50, 75, 100, 125, 150, 175, 200, 250, 300, 350, 400, or 500 nucleotides in length.

In embodiments, the oligonucleotide includes at least one target-specific region. In embodiments, the oligonucleotide includes two target-specific regions. In embodiments, the oligonucleotide includes at least one flanking-target region (i.e., an oligonucleotide sequence that flanks the region of interest). In embodiments, the oligonucleotide includes two flanking-target regions. A target-specific region is a single stranded polynucleotide that is at least 50% complementary, at least 75% complementary, at least 85% complementary, at least 90% complementary, at least 95% complementary, at least 98%, at least 99% complementary, or 100% complementary to a portion of a nucleic acid molecule that includes a target sequence (e.g., a gene of interest). In embodiments, the target-specific region is capable of hybridizing to at least a portion of the target sequence. In embodiments, the target-specific region is substantially non-complementary to other target sequences present in the sample. In embodiments, the oligonucleotide is a padlock probe. Padlock probes are specialized ligation probes, examples of which are known in the art, see for example Nilsson M, et al. Science. 1994; 265(5181):2085-2088), and has been applied to detect transcribed RNA in cells, see for example Christian A T, et al. PNAS. 2001; 98(25):14238-14243, both of which are incorporated herein by reference in their entireties.

Typically, padlock probes hybridize to adjacent sequences and are then ligated together to form a circular oligonucleotide. In embodiments, an oligonucleotide probe hybridizes to sequences adjacent to the target nucleic acid sequence resulting in a gap (e.g., a gap spanning the length of the target nucleic acid sequence). The construction of the oligonucleotide allows for selective targeting, enabling detection of specific targets on or within the cell or tissue section. In embodiments, the method further includes amplifying and sequencing the oligonucleotide.

In embodiments, the label is a fluorescent moiety that has a maximum excitation wavelength between 350-400 nm, between 400-450 nm, between 450-500 nm, between 500-550 nm, between 550-600 nm, between 600-650 nm, between 650-700 nm, or between 700-750 nm. In embodiments, the label is a fluorescent moiety that has a maximum emission wavelength between 400-450 nm, between 450-500 nm, between 500-550 nm, between 550-600 nm, between 600-650 nm, between 650-700 nm, between 700-750 nm, between 750-800 nm, or between 800-850 nm.

In embodiments, detecting a biomolecule in a cell or tissue includes detecting a plurality of different targets within an optically resolved volume of a cell or tissue immobilized onto the first solid support described herein. In embodiments, the method includes i) associating a different oligonucleotide barcode from a known set of barcodes with each of the plurality of targets; ii) sequencing each barcode to obtain a multiplexed signal in or on the cell or tissue; iii) demultiplexing the multiplexed signal by comparison with the known set of barcodes; and iv) detecting the plurality of targets by identifying the associated barcodes detected in or on the cell or tissue. In embodiments, the method includes detecting a plurality of targets (e.g., a nucleic acid sequence or a protein) within an optically resolved volume of a sample (e.g., a voxel). In embodiments, the method includes i) associating an oligonucleotide barcode with each of the plurality of targets; ii) sequencing each barcode to obtain a multiplexed signal; and iii) demultiplexing the multiplexed signal to obtain a set of signals corresponding to barcodes with a specified Hamming distance; thereby detecting a plurality of targets within an optically resolved volume of a sample.

In embodiments, detecting a biomolecule in a cell or tissue includes detecting a plurality of different nucleic acid sequences within an optically resolved volume of cell or tissue immobilized onto the first solid support described herein, wherein the method includes i) associating a different oligonucleotide barcode from a known set of barcodes with each of the plurality of targets, wherein associating an oligonucleotide barcode with each of the plurality of targets includes hybridizing a padlock probe to two adjacent nucleic acid sequences of the target, wherein the padlock probe is a single-stranded polynucleotide having a 5′ and a 3′ end, and wherein the padlock probe includes a primer binding sequence from a known set of primer binding sequences; ii) sequencing each barcode to obtain a multiplexed signal in or on the cell or tissue; iii) demultiplexing the multiplexed signal by comparison with the known set of barcodes; and iv) detecting the plurality of targets by identifying the associated barcodes detected in or on the cell.

In embodiments, detecting a biomolecule in a cell or tissue includes detecting a plurality of proteins (e.g., different proteins) within an optically resolved volume of a cell or tissue immobilized onto the first solid support described herein, wherein the method includes i) associating a different oligonucleotide barcode from a known set of barcodes with each of the plurality of targets, wherein associating an oligonucleotide barcode with each of the plurality of targets includes contacting each of the targets with a specific binding reagent, wherein the specific binding reagent includes an oligonucleotide barcode; ii) hybridizing a padlock probe to two adjacent nucleic acid sequences of the barcode, wherein the padlock probe is a single-stranded polynucleotide having a 5′ and a 3′ end, and wherein the padlock probe includes a primer binding sequence from a known set of primer binding sequences; iii) sequencing each barcode to obtain a multiplexed signal in or on the cell or tissue; iv) demultiplexing the multiplexed signal by comparison with the known set of barcodes; and v) detecting the plurality of targets by identifying the associated barcodes detected in or on the cell or tissue.

In embodiments, detecting a biomolecule in a cell or tissue includes detecting a plurality of proteins (e.g., different proteins) within an optically resolved volume of a cell or tissue immobilized onto the first solid support described herein, wherein the method includes i) associating a different oligonucleotide barcode from a known set of barcodes with each of the plurality of targets, wherein associating an oligonucleotide barcode with each of the plurality of targets includes contacting each of the targets with a specific binding reagent, wherein the specific binding reagent includes an oligonucleotide barcode; ii) sequencing each barcode to obtain a multiplexed signal in or on the cell or tissue; iii) demultiplexing the multiplexed signal by comparison with the known set of barcodes; and iv) detecting the plurality of targets by identifying the associated barcodes detected in or on the cell or tissue.

In embodiments, detecting a biomolecule in a cell or tissue includes a) detecting a plurality of proteins (e.g., different proteins), followed by b) detecting a plurality of different nucleic acid sequences within an optically resolved volume of cell or tissue immobilized onto the first solid support described herein. In embodiments, detecting a biomolecule in a cell or tissue includes a) detecting a plurality of nucleic acid sequences, followed by b) detecting a plurality of different proteins (e.g., different proteins within an optically resolved volume of cell or tissue immobilized onto the first solid support described herein.

In embodiments, the dimensions (i.e., the x, y, and z dimensions) of the optically resolved volume in a cell or tissue are about 0.5 μm×0.5 μm×0.5 m; 1 μm×1 μm×1 m; 2 μm×2 μm×2 m; 0.5 μm×0.5 μm×1 m; 0.5 μm×0.5 μm×2 m; 2 μm×2 μm×1 m; or 1 μm×1 μm×2 μm.

In embodiments, the method further includes amplifying a nucleic acid molecule (e.g., a nucleic acid molecule in a cell) to generate amplification products. In embodiments, amplifying includes contacting the flow cell assembly as described herein with one or more reagents for amplifying the target polynucleotide. Examples of reagents include but are not limited to polymerase, buffer, and nucleotides (e.g., an amplification reaction mixture). In certain embodiments the term “amplifying” refers to a method that includes a polymerase chain reaction (PCR). Conditions conducive to amplification (i.e., amplification conditions) are known and often include at least a suitable polymerase, a suitable template, a suitable primer or set of primers, suitable nucleotides (e.g., dNTPs), a suitable buffer, and application of suitable annealing, hybridization and/or extension times and temperatures. In embodiments, amplifying generates an amplicon. In embodiments, amplifying generates a rolony. In embodiments, an amplicon contains multiple, tandem copies of the circularized nucleic acid molecule of the corresponding sample nucleic acid. The number of copies can be varied by appropriate modification of the amplification reaction including, for example, varying the number of amplification cycles run, using polymerases of varying processivity in the amplification reaction and/or varying the length of time that the amplification reaction is run, as well as modification of other conditions known in the art to influence amplification yield. Generally, the number of copies of a nucleic acid in an amplicon is at least 100, 200, 500, 1000, 2000, 3000, 4000, 5000, 6000, 7000, 8000, 9000 and 10,000 copies, and can be varied depending on the application. As disclosed herein, one form of an amplicon is as a nucleic acid “ball” localized to the particle and/or well of the array. The number of copies of the nucleic acid can therefore provide a desired size of a nucleic acid “ball” or a sufficient number of copies for subsequent analysis of the amplicon, e.g., sequencing.

In embodiments, amplifying includes bridge polymerase chain reaction (bPCR) amplification, solid-phase rolling circle amplification (RCA), solid-phase exponential rolling circle amplification (eRCA), solid-phase recombinase polymerase amplification (RPA), solid-phase helicase dependent amplification (HDA), template walking amplification, or emulsion PCR on particles, or combinations of the methods. In embodiments, amplifying includes a bridge polymerase chain reaction amplification. In embodiments, amplifying includes a thermal bridge polymerase chain reaction (t-bPCR) amplification. In embodiments, amplifying includes a chemical bridge polymerase chain reaction (c-bPCR) amplification. Chemical bridge polymerase chain reactions include fluidically cycling a denaturant (e.g., formamide) and one or more additives (e.g., ethylene glycol) and maintaining the temperature within a narrow temperature range (e.g., +/−5° C.) or isothermally. In embodiments, c-bPCR does not include isothermal amplification, rather it requires minor (e.g., +/−5° C.) thermal oscillations. In contrast, thermal bridge polymerase chain reactions include thermally cycling between high temperatures (e.g., 85° C.-95° C.) and low temperatures (e.g., 60° C.-70° C.). Thermal bridge polymerase chain reactions may also include a denaturant, typically at a much lower concentration than traditional chemical bridge polymerase chain reactions. In embodiments, amplifying includes generating a double-stranded amplification product.

It will be appreciated that any of the amplification methodologies described herein or known in the art can be utilized with universal or target-specific primers to amplify the target polynucleotide. Suitable methods for amplification include, but are not limited to, the polymerase chain reaction (PCR), strand displacement amplification (SDA), transcription mediated amplification (TMA) and nucleic acid sequence-based amplification (NASBA), for example, as described in U.S. Pat. No. 8,003,354, which is incorporated herein by reference in its entirety. The above amplification methods can be employed to amplify one or more nucleic acids of interest. Additional examples of amplification processes include, but are not limited to, bridge-PCR, recombinase polymerase amplification (RPA), loop-mediated isothermal amplification (LAMP), rolling circle amplification (RCA), strand displacement amplification (SDA), rolling circle amplification (RCA) with exponential strand displacement amplification. In embodiments, amplification includes an isothermal amplification reaction. In embodiments, amplification includes bridge amplification. In general, bridge amplification uses repeated steps of annealing of primers to templates, primer extension, and separation of extended primers from templates. Because primers are attached within the core polymer, the extension products released upon separation from an initial template is also attached within the core. The 3′ end of an amplification product is then permitted to anneal to a nearby reverse primer that is also attached within the core, forming a “bridge” structure. The reverse primer is then extended to produce a further template molecule that can form another bridge. In embodiments, forward and reverse primers hybridize to primer binding sites that are specific to a particular target nucleic acid. In embodiments, forward and reverse primers hybridize to primer binding sites that have been added to, and are common among, target polynucleotides. Adding a primer binding site to target nucleic acids can be accomplished by any suitable method, examples of which include the use of random primers having common 5′ sequences and ligating adapter nucleotides that include the primer binding site. Examples of additional clonal amplification techniques include, but are not limited to, bridge PCR, solid-phase rolling circle amplification (RCA), solid-phase exponential rolling circle amplification, solid-phase recombinase polymerase amplification (RPA), solid-phase helicase dependent amplification (HDA), template walking amplification, emulsion PCR on particles (beads), or combinations of the aforementioned methods. Optionally, during clonal amplification, additional solution-phase primers can be supplemented in the microplate for enabling or accelerating amplification.

In embodiments, the amplifying includes rolling circle amplification (RCA) or rolling circle transcription (RCT) (see, e.g., Lizardi et al., Nat. Genet. 19:225-232 (1998), which is incorporated herein by reference in its entirety). Several suitable rolling circle amplification methods are known in the art. For example, RCA amplifies a circular polynucleotide (e.g., DNA) by polymerase extension of an amplification primer complementary to a portion of the template polynucleotide. This process generates copies of the circular polynucleotide template such that multiple complements of the template sequence arranged end to end in tandem are generated (i.e., a concatemer) locally preserved at the site of the circle formation. In embodiments, the amplifying occurs at isothermal conditions. In embodiments, the amplifying includes hybridization chain reaction (HCR). HCR uses a pair of complementary, kinetically trapped hairpin oligomers to propagate a chain reaction of hybridization events, as described in Dirks, R. M., & Pierce, N. A. (2004) PNAS USA, 101(43), 15275-15278, which is incorporated herein by reference for all purposes. In embodiments, the amplifying includes branched rolling circle amplification (BRCA); e.g., as described in Fan T, Mao Y, Sun Q, et al. Cancer Sci. 2018; 109:2897-2906, which is incorporated herein by reference in its entirety. In embodiments, the amplifying includes hyberbranched rolling circle amplification (HRCA). Hyperbranched RCA uses a second primer complementary to the first amplification product. This allows products to be replicated by a strand-displacement mechanism, which yields drastic amplification within an isothermal reaction (Lage et al., Genome Research 13:294-307 (2003), which is incorporated herein by reference in its entirety). In embodiments, amplifying includes polymerase extension of an amplification primer. In embodiments, the polymerase is T4, T7, Sequenase, Taq, Klenow, and Pol I DNA polymerases. SD polymerase, Bst large fragment polymerase, or a phi29 polymerase or mutant thereof. In embodiments, the strand-displacing enzyme is an SD polymerase, Bst large fragment polymerase, or a phi29 polymerase or mutant thereof. In embodiments, the strand-displacing polymerase is phi29 polymerase, phi29 mutant polymerase or a thermostable phi29 mutant polymerase. A “phi polymerase” (or “Φ29 polymerase”) is a DNA polymerase from the Φ29 phage or from one of the related phages that, like Φ29, contain a terminal protein used in the initiation of DNA replication.

In embodiments, the method further includes detecting the amplification products. In embodiments, detecting the amplification products includes detecting the label (e.g., the nucleic acid sequence). In embodiments, detecting the amplification products includes detecting the oligonucleotide label. In embodiments, detecting includes sequencing. In embodiments, sequencing includes extending a sequencing primer annealed to the target polynucleotide to incorporate a nucleotide containing a detectable label that indicates the identity of a nucleotide in the target polynucleotide, detecting the detectable label, and optionally repeating the extending and detecting of steps. In embodiments, the methods include sequencing one or more bases of a target nucleic acid by extending a sequencing primer hybridized to a target nucleic acid (e.g., an amplification product of a target nucleic acid). In embodiments, the sequencing includes sequencing-by-synthesis, sequencing by ligation, sequencing-by-hybridization, or pyrosequencing, and generates a sequencing read. In embodiments, generating a sequencing read includes executing a plurality of sequencing cycles, each cycle including extending the sequencing primer by incorporating a nucleotide or nucleotide analogue using a polymerase and detecting a characteristic signature indicating that the nucleotide or nucleotide analogue has been incorporated.

In embodiments, sequencing includes a plurality of sequencing cycles. In embodiments, sequencing includes 20 to 100 sequencing cycles. In embodiments, sequencing includes 50 to 100 sequencing cycles. In embodiments, sequencing includes 50 to 300 sequencing cycles. In embodiments, sequencing includes 50 to 150 sequencing cycles. In embodiments, sequencing includes at least 10, 20, 30 40, or 50 sequencing cycles. In embodiments, sequencing includes at least 10 sequencing cycles. In embodiments, sequencing includes 10 to 20 sequencing cycles. In embodiments, sequencing includes 10, 11, 12, 13, 14, or 15 sequencing cycles. In embodiments, sequencing includes (a) extending a sequencing primer by incorporating a labeled nucleotide, or labeled nucleotide analogue and (b) detecting the label to generate a signal for each incorporated nucleotide or nucleotide analogue.

In embodiments, the method includes sequencing the first and/or the second strand of a amplification product by extending a sequencing primer hybridized thereto. A variety of sequencing methodologies can be used such as sequencing-by-synthesis (SBS), pyrosequencing, sequencing by ligation (SBL), or sequencing by hybridization (SBH). Pyrosequencing detects the release of inorganic pyrophosphate (PPi) as particular nucleotides are incorporated into a nascent nucleic acid strand (Ronaghi, et al., Analytical Biochemistry 242(1), 84-9 (1996); Ronaghi, Genome Res. 11(1), 3-11 (2001); Ronaghi et al. Science 281(5375), 363 (1998); U.S. Pat. Nos. 6,210,891; 6,258,568; and. 6,274,320, each of which is incorporated herein by reference in its entirety). In pyrosequencing, released PPi can be detected by being converted to adenosine triphosphate (ATP) by ATP sulfurylase, and the level of ATP generated can be detected via light produced by luciferase. In this manner, the sequencing reaction can be monitored via a luminescence detection system. In both SBL and SBH methods, target nucleic acids, and amplicons thereof, that are present at features of an array are subjected to repeated cycles of oligonucleotide delivery and detection. SBL methods, include those described in Shendure et al. Science 309:1728-1732 (2005); U.S. Pat. Nos. 5,599,675; and 5,750,341, each of which is incorporated herein by reference in its entirety; and the SBH methodologies are as described in Bains et al., Journal of Theoretical Biology 135(3), 303-7 (1988); Drmanac et al., Nature Biotechnology 16, 54-58 (1998); Fodor et al., Science 251(4995), 767-773 (1995); and WO 1989/10977, each of which is incorporated herein by reference in its entirety.

In SBS, extension of a nucleic acid primer along a nucleic acid template is monitored to determine the sequence of nucleotides in the template. The underlying chemical process can be catalyzed by a polymerase, wherein fluorescently labeled nucleotides are added to a primer (thereby extending the primer) in a template dependent fashion such that detection of the order and type of nucleotides added to the primer can be used to determine the sequence of the template. A plurality of different nucleic acid fragments can be subjected to an SBS technique under conditions where events occurring for different templates can be distinguished due to their location in the array. In embodiments, the sequencing step includes annealing and extending a sequencing primer to incorporate a detectable label that indicates the identity of a nucleotide in the target polynucleotide, detecting the detectable label, and repeating the extending and detecting steps. In embodiments, the methods include sequencing one or more bases of a target nucleic acid by extending a sequencing primer hybridized to a target nucleic acid (e.g., an amplification product produced by the amplification methods described herein). In embodiments, the sequencing step may be accomplished by an SBS process. In embodiments, sequencing includes a sequencing by synthesis process, where individual nucleotides are identified iteratively, as they are polymerized to form a growing complementary strand. In embodiments, nucleotides added to a growing complementary strand include both a label and a reversible chain terminator that prevents further extension, such that the nucleotide may be identified by the label before removing the terminator to add and identify a further nucleotide. Such reversible chain terminators include removable 3′ blocking groups, for example as described in U.S. Pat. No. 10,738,072 or 11,174,281. Once such a modified nucleotide has been incorporated into the growing polynucleotide chain complementary to the region of the template being sequenced, there is no free 3′-OH group available to direct further sequence extension and therefore the polymerase cannot add further nucleotides. Once the identity of the base incorporated into the growing chain has been determined, the 3′ block may be removed to allow addition of the next successive nucleotide. By ordering the products derived using these modified nucleotides it is possible to deduce the DNA sequence of the DNA template. Non-limiting examples of suitable labels are described in U.S. Pat. Nos. 8,178,360, 5,188,934 (4,7-dichlorofluorscein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthene dyes): U.S. Pat. No. 5,688,648 (energy transfer dyes); and the like.

Sequencing includes, for example, detecting a sequence of signals. Examples of sequencing include, but are not limited to, sequencing by synthesis (SBS) processes in which reversibly terminated nucleotides carrying fluorescent dyes are incorporated into a growing strand, complementary to the target strand being sequenced. In embodiments, the nucleotides are labeled with up to four unique fluorescent dyes. In embodiments, the nucleotides are labeled with at least two unique fluorescent dyes. In embodiments, the readout is accomplished by epifluorescence imaging. A variety of sequencing chemistries are available, non-limiting examples of which are described herein.

Use of the sequencing method outlined above is a non-limiting example, as essentially any sequencing methodology which relies on successive incorporation of nucleotides into a polynucleotide chain can be used. Suitable alternative techniques include, for example, pyrosequencing methods, FISSEQ (fluorescent in situ sequencing), MPSS (massively parallel signature sequencing), or sequencing by ligation-based methods.

In embodiments, detecting includes detecting 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or 3 transcripts per μm². In embodiments, detecting includes detecting 4, 5, 6, 7, 8, 9, 10, 11, 12, 13, 14, 15, 16, 17, 18, 19, 2, 21, 22, 23, 24, 25, 26, 27, 28, 29, or 30 transcripts per μm². In embodiments, detecting includes detecting 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or 3 transcripts per μm³. In embodiments, detecting includes detecting 4, 5, 6, 7, 8, 9, 10, 11, 12, 13, 14, 15, 16, 17, 18, 19, 2, 21, 22, 23, 24, 25, 26, 27, 28, 29, or 30 transcripts per μm³.

In embodiments, the method further includes obtaining an image of a cell or tissue. In embodiments, the imaging includes phase-contrast microscopy, bright-field microscopy, Nomarski differential-interference-contrast microscopy, dark field microscopy, electron microscopy, or cryo-electron microscopy. In embodiments, the light transmittance of the sample is measured. For example, light transmittance may be measured with a visible near-infrared optical fiber spectrometer, wherein a circular spot of light (e.g., diameter, 5 mm) is irradiated on the central part a sample and the transmitted light is collected using an optical sensor.

In embodiments, the method further includes an imaging modality including immunofluorescence (IF), or immunohistochemistry modality (e.g., immunostaining). In embodiments, the method further includes an imaging modality including fluorescent hematoxylin and eosin (H&E) modality. For example, the method includes contacting the sample with a stain (e.g., Acridine orange, Auramine O, Calcofluor white, DAPI, Ethidium bromide, Hoechst 33258, Propidium iodide, Rhodamine B, SYBR Green, Texas Red, Thioflavin T, TOTO®-3, Uvitex 2B, YOYO®-1, 7-Aminoactinomycin D (7-AAD), TO-PRO®, TOPRO-3®, or eosin). In embodiments, the method includes ER staining (e.g., contacting the tissue section with a cell-permeable dye which localizes to the endoplasmic reticula), Golgi staining (e.g., contacting the tissue section with a cell-permeable dye which localizes to the Golgi), F-actin staining (e.g., contacting the tissue section with a phalloidin-conjugated dye that binds to actin filaments), lysosomal staining (e.g., contacting the tissue section with a cell-permeable dye that accumulates in the lysosome via the lysosome pH gradient), mitochondrial staining (e.g., contacting the tissue section with a cell-permeable dye which localizes to the mitochondria), nucleolar staining, or plasma membrane staining. For example, the method includes live cell imaging (e.g., obtaining images of the tissue section) prior to or during fixing, immobilizing, and permeabilizing the tissue section. Immunohistochemistry (IHC) is a powerful technique that exploits the specific binding between an antibody and antigen to detect and localize specific antigens in cells and tissue, commonly detected and examined with the light microscope. Known IHC modalities may be used, such as the protocols described in Magaki, S., Hojat, S. A., Wei, B., So, A., & Yong, W. H. (2019). Methods in molecular biology (Clifton, N.J.), 1897, 289-298, which is incorporated herein by reference. In embodiments, the additional imaging modality includes bright field microscopy, phase contrast microscopy, Nomarski differential-interference-contrast microscopy, or dark field microscopy. In embodiments, the method further includes determining the cell morphology of the tissue section (e.g., the cell boundary or cell shape) using known methods in the art. For example, to determining the cell boundary includes comparing the pixel values of an image to a single intensity threshold, which may be determined quickly using histogram-based approaches as described in Carpenter, A. et al Genome Biology 7, R100 (2006) and Arce, S., Sci Rep 3, 2266 (2013)). By “microscopic analysis” is meant the analysis of a specimen using techniques that provide for the visualization of aspects of a specimen that cannot be seen with the unaided eye, i.e., that are not within the resolution range of the normal human eye. Such techniques may include, without limitation, optical microscopy, e.g., bright field, oblique illumination, dark field, phase contrast, differential interference contrast, interference reflection, epifluorescence, confocal microscopy, CLARITY-optimized light sheet microscopy (COLM), light field microscopy, tissue expansion microscopy, etc., laser microscopy, such as, two photon microscopy, electron microscopy, and scanning probe microscopy. By “preparing a biological specimen for microscopic analysis” is generally meant rendering the specimen suitable for microscopic analysis at an unlimited depth within the specimen. In embodiments, the immobilized tissue section is imaged using “optical sectioning” techniques, such as laser scanning confocal microscopes, laser scanning 2-Photon microscopy, parallelized confocal (i.e. spinning disk), computational image deconvolution methods, and light sheet approaches. Optical sectioning microscopy methods provide information about single planes of a volume by minimizing contributions from other parts of the volume and do so without physical sectioning. The resulting “stack” of such optically sectioned images, represents a full reconstruction of the 3-dimensional features of a tissue volume. A typical confocal microscope includes a 10×/0.5 objective (dry; working distance, 2.0 mm) and/or a 20×/0.8 objective (dry; working distance, 0.55 mm), with a s z-step interval of 1 to 5 m. A typical light sheet fluorescence microscope includes an sCMOS camera, a 2×/0.5 objective lens, and zoom microscope body (magnification range of ×0.63 to ×6.3). For entire scanning of whole samples, the z-step interval is 5 or 10 m, and for image acquisition in the regions of interest, an interval in the range of 2 to 5 m may be used.

In embodiments, the imaging modality is capable of imaging an imaging area of about 1 cm² to about 10 cm², 1 cm² to about 5 cm², 5 cm² to about 10 cm², 10 cm² to about 30 cm², 30 cm² to about 60 cm², 60 cm² to about 90 cm², 90 cm² to about 120 cm², or more.

In embodiments, the collection of information (e.g., sequencing information and cell morphology) is referred to as a signature. The term “signature” may encompass any gene or genes, protein or proteins, or epigenetic element(s) whose expression profile or whose occurrence is associated with a specific cell type, subtype, or cell state of a specific cell type or subtype within a population of cells. It is to be understood that also when referring to proteins (e.g., differentially expressed proteins), such may fall within the definition of “gene” signature. Levels of expression or activity or prevalence may be compared between different cells in order to characterize or identify for instance signatures specific for cell (sub)populations. Increased or decreased expression or activity of signature genes may be compared between different cells in order to characterize or identify for instance specific cell (sub)populations.

In embodiments, the methods described herein may further include constructing a 3-dimensional pattern of abundance, expression, and/or activity of each target from spatial patterns of abundance, expression, and/or activity of each target of multiple samples. In embodiments, the multiple samples can be consecutive tissue sections of a 3-dimensional tissue sample.

### Examples

### Example 1. Development of Flow Cell Assembly for Robust Tissue Adhesion

The compatibility of surfaces with specific biological sample types is often not universal among different sample types, such as the case where a surface (e.g., a slide) is suitable for formalin-fixed paraffin-embedded (FFPE) tissue but not for other types like fresh frozen, paraformaldehyde (PFA)-fixed frozen tissue, or fresh cultured cells. Additionally, surfaces of slides used in histology and pathology often require some level of customization based on the tissue source, like kidney, bone marrow, tonsil, or liver. Specific tissue types exhibit a heightened propensity for detachment from slides, including skin, bone, cartilage, dental tissue, and retinal tissue, as well as biopsies of fatty tissues such as the brain. Samples that are cut to thicknesses deviating from standard parameters, nerve cells, or specimens including multiple tissue types are also prone to separation from the slide surface. Different adhesives used in slide coating provide different degrees of hydrophobicity, hydrophilicity, wettability and contact angle tailored for particular tissue types and applications.

Typical sample slides are substantially planar (i.e., flat) glass, often 75×25 mm and 1 mm thick, or 3×1-inch and 1 mm thick, and are used to hold a biological specimen. In histopathology applications the glass slides are ground and polished for safe handling and include a frosted area painted for labeling purposes. Distinguishing between glass utilized in enological applications (wine glasses) and glass employed in histological laboratories, it is pertinent to note the compositional differences. In histological applications, borosilicate glass is predominantly utilized, attributed to its superior properties concerning its transmittance and reflectivity properties. Borosilicate glass is characterized by its high clarity and minimal light absorption, allowing for the transmission of a greater spectrum of light. Such a property is essential in microscopy, as it ensures that more light passes through the specimen, thereby providing clearer, more detailed visualizations of the sample under observation. Enhanced light transmittance is crucial for accurate and detailed microscopic examinations, particularly in high-resolution imaging. Additionally, the reduced reflectivity of borosilicate glass minimizes the interference caused by surface reflections, thus enhancing the quality of the image. Low reflectivity is especially beneficial when examining specimens that require high magnification or intricate detail observation, as it ensures that the light is focused on the specimen rather than being reflected off the surface. The absence of additives in borosilicate glass during the manufacturing process contributes to its enhanced purity, yielding superior optical and thermal characteristics. Consequently, this renders borosilicate glass more costly.

Silica, the foundational constituent of glass, features a tetrahedral arrangement where each silicon atom is covalently bonded to four oxygen atoms. This robust lattice structure imparts glass with its relatively inert nature, as the preoccupied valence electrons in the silicon-oxygen bonds leave minimal propensity for additional chemical interactions. Due to the inertness of bare glass (i.e., unmodified glass), which lack chemical coatings, a risk of sample loss may exist due to the absence of chemical adhesion between the tissue and the glass surface. To mitigate this issue, histological laboratories often resort to heating techniques, wherein tissue samples are essentially baked onto the glass slide. This process also leverages the evaporation of water trapped between the tissue and the glass, aiding in the retention of the sample on the slide.

Immunohistochemistry commonly subjects tissue samples to elevated temperatures and numerous washing cycles with buffers of varying pH, alongside antigen retrieval methods that employ high temperatures, pressures, enzymes, or chemicals. Similar to cleaning dishware in a dishwasher, similar processes can lead to deformation and dislodgement of tissue from glass slides. Similarly, in situ hybridization techniques involve high temperatures and prolonged stringency washes, potentially endangering tissue integrity if non-coated slides are used. To mitigate these risks, employing slides with enhanced adhesive properties is commonly utilized to safeguard the specimen. These adhesives interact with hydroxyl groups present on the glass surface, subsequently exposing amino groups that bear a positive charge. Given that most animal tissues present a net negative charge, they are naturally attracted to the positively charged amino groups on the slide, forming strong electrostatic bonds. These bonds are significantly stronger, ensuring robust attachment of the tissue to the slide, thereby preserving the integrity of the specimen during the histological processing.

Common additives on slides include proteins like albumin, gelatin, Poly-L-lysine, and extracts from the Cordia myxa tree, which provide amine groups (i.e., —NH₂) groups to attract negatively charged tissue samples. Some have historically used protein-based additives like Elmer's Glue or gelatin, however, such additives can create background staining due to off-target hematoxylin binding to large proteins within the additive. Commercial slide coatings include aminosilanes, such as 3-Aminopropyltriethoxysilane (APTES) or 3-Aminopropyltrimethoxysilane (APTMS), which are compounds where an amine group is bonded to a silicon atom. The diversity among the approximately 3,000 aminosilanes can be attributed to differences in the length of the carbon chain, the presence of additional functional groups, and the degree of branching. Furthermore, the choice of aminosilane can influence the reactivity towards other chemicals used in slide preparation and staining processes. For instance, certain aminosilanes may provide enhanced reactivity towards specific tissue components, facilitating stronger adhesion. Polyethyleneimine (PEI) is another common surface coating containing primary, secondary and tertiary amino groups in 1:2:1 ratio. PEI-coated surfaces exhibit inconsistent performance across different tissue types. Surface variability is due in part to the random orientation of PEI during the bonding process, resulting in the diverse amine groups being directed both towards the surface and the tissue. Consequently, the availability of functional groups for tissue bonding is reduced, leading to inconsistency in surface behavior.

Most NGS devices utilize a flow cell to capture nucleic acid fragments, amplify the fragments, and sequence the fragments. For example, the Singular Genomics flow cell, similar to a microscope slide, is made of an optically transparent surface coated with oligonucleotides which are complementary to the sequencing adapters so that single-stranded, adapter-ligated DNA fragments can attach through hybridization. After attaching a DNA template to the anchors, solid phase amplification methods may be used to generate millions of copies of the template. While flow cells have been instrumental in transforming the efficiency and economic feasibility of NGS DNA sequencing, their application in spatial biology, particularly for in situ RNA detection within cells and tissues, remains unexplored and challenging. A primary impediment in this translation is tissue delamination, a process where the structural integrity of tissue samples is compromised during repetitive reagent exchanges. Furthermore, when utilizing tissue sections, commonly around 5 μm in thickness, additional complications arise. Thin tissue sections are prone to wrinkling, which leads to non-uniform exposure to reagents and target detection inefficiencies. Degradation can result from both the mechanical handling of these fragile sections and the chemical interactions during the sequencing process, leading to a loss of vital biological information and potential misinterpretation of sequencing data. The development of a spatial in-situ sequencing platform would enable leveraging novel sequencing chemistry, fast optics, and innovative engineering that is foundational to commercially available Next Generation Sequencing (NGS) benchtop sequencers, such as the G4X™ provided by Singular Genomics. However, the lack of such spatial in-situ sequencing platform where spatial biology analyses are performed directly on a flow cell remains unexplored and such deficiency prevents the efficiency provided from using flow cells to be realized for spatial biology applications.

### Example 2. Polymer Surface Functionalization

The adhesion of tissue sections to solid supports, such as glass slides, is fundamental for biomedical imaging modalities that provide insights into the composition, architecture, abundance, and spatial distribution of key macromolecules. Common workflows for detecting nucleic acids and protein molecules in tissue sections often require harsh conditions, including high temperatures and extended incubation times, which can lead to tissue detachment from the slides, a phenomenon known as tissue delamination. Additionally, tissue type significantly influences its adherence to glass slides; for instance, tissues derived from skin, bone, and brain are particularly prone to delamination, posing critical challenges for their manipulation and analysis. Furthermore, the structure and morphology of tissue sections can be altered under assay conditions, resulting in tissue distortion.

Surface functionalization methods for glass tissue slides are known. Organosilane compounds are commonly used to modify the surface of glass slides with reactive chemical moieties that enhance cell adhesion between the glass and the functional groups present in tissue sections. However, recent studies have highlighted limitations in these methods. Das et al. reported that biomolecules bound to silane-functionalized glass surfaces can detach during high-throughput genomic workflows, which often involve high temperatures and prolonged incubation times (Anal. Chem. 2023, 95, 41, 15384-15393). They also noted that such assay conditions promote hydrolytic instability of the Si—O—Si bond formed between the hydroxyl groups of the glass slide and the silane moieties of the organosilane compound. Additionally, certain surface functionalization agents, such as PEG-oxysilane, have been shown to inhibit rather than promote cell adhesion (Chen et al. Langmuir. 2010 Dec. 7; 26(23):17790-4).

Moreover, we have previously observed that functionalizing the surface of glass tissue slides with the aminosilane compound (3-Aminopropyl)triethoxysilane (APTES) or PEI resulted in inconsistent tissue adhesion under conditions used for spatial transcriptomics studies. This highlights the critical need for developing a robust glass substrate that can consistently adhere to tissue sections and maintain this adhesion throughout various workflows.

Described herein are compositions and methods aimed at developing a robust flow cell assembly, including a functionalized glass tissue slide, that facilitates reliable tissue adherence throughout amplification and sequencing workflows. The selection of a suitable polymer formulation is guided by the need to balance immediate tissue adhesion with long-term stability under demanding processing conditions. For example, the polymer formulation should facilitate attachment to various tissue types, does not interfere with sequencing reagents and detection of detectable labels used in sequencing, resistant to high temperatures (e.g., temperatures used during antigen retrieval and sequencing workflows), and stable at room temperature storage conditions.

We sought to create a polymer with amines oriented substantially away from the surface so as to maximize their contact with the tissue. Acrylamide and PEI coatings do not maximize the amount of amine moieties available for bonding to the tissue, where a substantial fraction bind to the surface. Therefore, we designed a polymer that includes a hydrophobic component (e.g., acryl chain) and includes a protected amine group (see FIG. 1), such that during surface coating the hydrophobic component binds to the surface and orients the protected amine moieties normal to the surface (see FIG. 2A). Removal of the protection group renders the free amine moiety to be available for tissue bonding (see FIG. 2B). The inclusion of both a hydrophobic component and the protected amine ensures that the polymer can withstand the physical and chemical stresses encountered during these processes, maintaining tissue integrity and preventing delamination. To prove out the design, we generated Poly(ethylene glycol) methacrylate azides (PEGMA-N3) in solution, coated the surface of a glass slide, and reduced the azide moieties to provide amine moieties.

In embodiments, the solid support (e.g., glass slide) included a resist (e.g., a nanoimprint lithography (NIL) resist). Nanoimprint resists can include thermal curable materials (e.g., thermoplastic polymers), and/or UV-curable polymers. For example, the solid support surface is coated in an organically modified ceramic polymer (ORMOCER®, registered trademark of Fraunhofer-Gesellschaft zur Förderung der angewandten Forschung e. V. in Germany). Organically modified ceramics contain organic side chains attached to an inorganic siloxane backbone. Several ORMOCER® polymers are now provided under names such as “Ormocore”, “Ormoclad” and “Ormocomp” by Micro Resist Technology GmbH. In embodiments, the solid support includes a resist as described in Haas et al Volume 351, Issues 1-2, 30 Aug. 1999, Pages 198-203, US 2015/0079351A1, US 2008/0000373, US 2010/0160478, or U.S. Pat. No. 10,268,096 B2, each of which is incorporated herein by reference. In embodiments, the solid support surface is coated in an organically modified ceramic polymer including (ORMOCER®, registered trademark of Fraunhofer-Gesellschaft zur Förderung der angewandten Forschung e. V. in Germany). Ormocomp®, an organically modified ceramic, provides a stable and reactive surface that may enhance the adhesion of the hydrogel to the glass silica slide. The ceramic coating interacts with the functional groups in the hydrogel, forming a robust base that supports both non-covalent and covalent bonding mechanisms.

Synthesis of PEGMA-N3. To a clean 500 mL flask, polyethylene glycol (average Mn=400) was added. While stirring, 4-(dimethylamino)pyridine was added to the reaction mixture, along with triethylamine. Next, 4-toluenesulfonyl chloride was added and stirred overnight. The reaction mixture was extracted in dichloromethane and evaporated using a rotary evaporator to generate a monotosylated-PEG. The monotosylated PEG was diluted in DML and sodium azide was added and sealed in a flask. The flask was heated above 80° C. and stirred 24 hours. The reaction was diluted with 150 mL of DI water and cooled to room temperature. The solution was filtered and washed using dichloromethane to provide the compound an azido PEG:

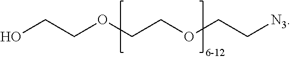

The azido polyethylene glycol (N3-PEG-OH) was added to a round bottom flask, along with 100 mL of dichloromethane and N-diisopropylethylamine (6 mL) and set over ice. Methacryloyl chloride was added slowly to the reaction mixture. The resulting product,

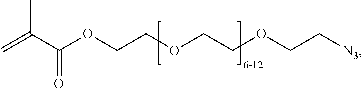

was washed and isolated using a rotary evaporator.

Atom transfer radical polymerization of PEGMA-N3. The PEGMA-N3 was added to a flask, along with, OH-EBiB, HMTETA, in DMF and ethanol and mixed at 35° C. The flask was stirred in an ice bath for 30, followed by degassing and adding copper bromide. The reaction mixture was stirred overnight. In a second flask, n-hexane was stirred and the entire polymer mixture was added dropwise and stirred for about 25 minutes. The polymer precipitated out of the n-hexane solution. The resulting linear polymer,

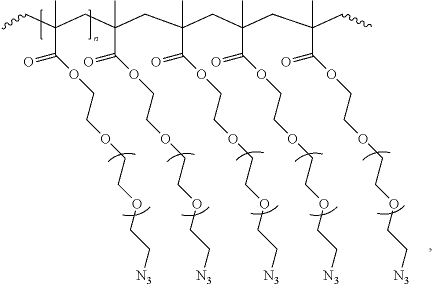

N was washed and isolated using a rotary evaporator.

Slide incubation in polyPEGMA-N₃ solution. Borosilicate glass slides were washed three times in ethanol bath while being sonicated. The glass slides were submerged into Ormocomp® (Micro Resist Technology GmbH, Germany), then contacted with Ormodev solvent (50:50 4-methyl-2-pentanone and 2-propanol, Micro Resist Technology GmbH, Germany), and rinsed using hexamethyldisilazane (HMDS). The control slides were further functionalized with an amine-containing polymer (PEI). The polymer solution was diluted in DI water at 1% wt. Glass slides coated with an organically modified ceramic (e.g., Ormocomp®) polymer resist were placed in the diluted polymer solution and incubated overnight. The polyPEGMA-N₃ coated slides were removed and rinsed with DI water.

Deprotection of the protected amine. The polyPEGMA-N₃ coated slides were placed in a jar with 1 mM TCEP (0.1M) and incubated overnight at room temperature. The polyPEGMA-NH2 coated slides were removed and rinsed with DI water and ethanol and dried. The slides were stored at room temperature

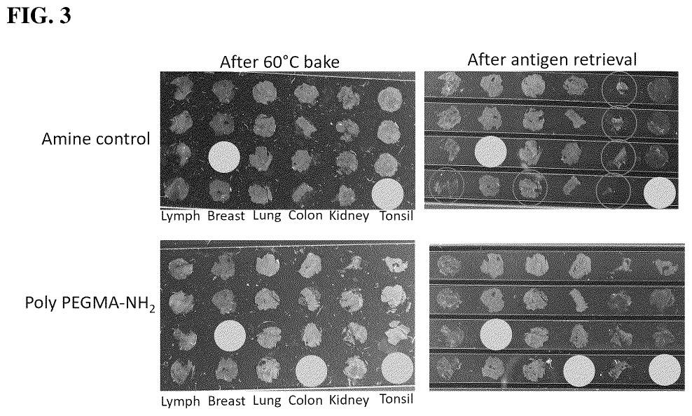

Deparaffinization and Heat-Induced Antigen Retrieval. FFPE tissue sections were prepared and transferred to the functionalized glass slide using techniques known in the art (see, e.g., PCT Publication WO2023076832A1). We tested multiple types of tissue, from fattier to more muscular types of tissue to ensure that the surface chemistry works universally. The tissues tested include lymph, breast, lung, colon, kidney, and tonsil. Following tissue section transfer, the slides were baked at elevated temperatures (e.g., 30-70° C.) and placed in dark storage at room temperature overnight. The tissue sections were then deparaffinized using xylene followed by 100% EtOH incubation. Following deparaffinization, the slides were immersed into antigen retrieval buffer (pH 9) and incubated in a pressure cooker. Comparison of tissue adherence to solid supports including a polymer layer and an amine-containing polymer control or the polymer composition is shown in FIG. 3. The surface of the solid supports were functionalized with Ormocomp® prior to the deposition of an amine-containing polymer control or the polymer polyPEGMA-NH2. Images of the tissue sections were obtained following transfer and baking at 60° C. and after antigen retrieval, as depicted in FIG. 3. The solid circles in FIG. 3 indicate poor initial tissue transfer (i.e., the tissues folded and ripped). The open circle indicate that after antigen retrieval, a majority of the kidney tissues, lung, and lymph partially or completely disintegrated from the amine control slide. In contrast, all tissue types remained attached to the solid support that contained a poly PEGMA-NH2 coating.

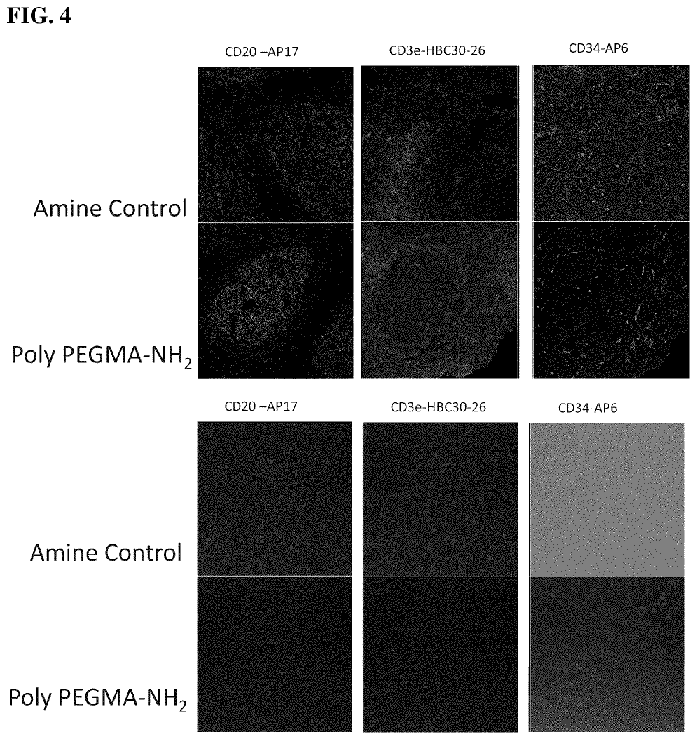

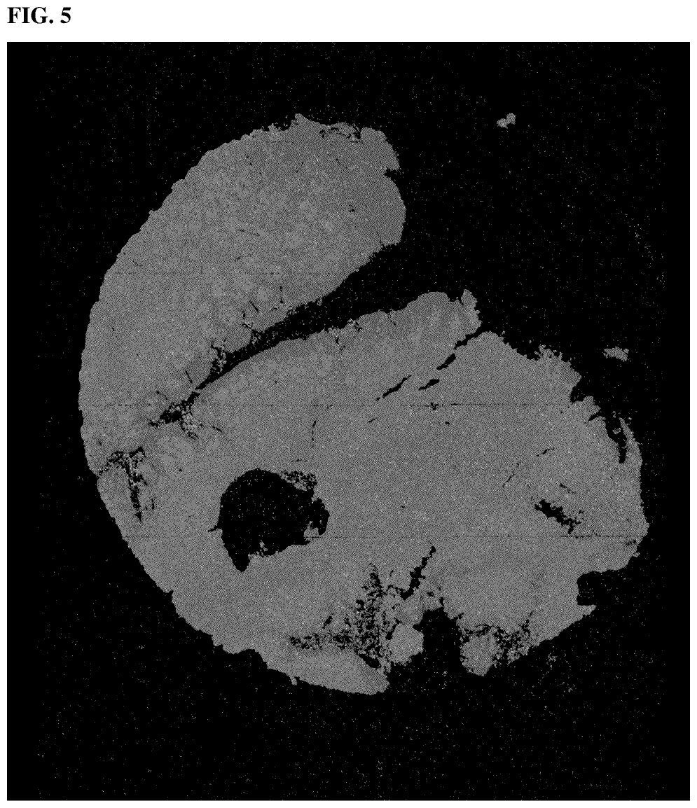

To evaluate whether the different formulations had an effect on signal intensity after standard processing, the tonsil-containing lane was further processed imaged. We used CD20, CD3e, and CD34 antibodies, each including a specific oligonucleotide conjugated, forming an antibody-oligo (Ab-O) conjugate. Following incubation with the antibodies, labeled probes specific for each oligonucleotide were bound and detected. FIG. 4 shows each antibody detected (top) wherein both surfaces provide suitable surfaces for detection. Low background signal is crucial in fluorescent detection as it ensures the accuracy and clarity of imaging results, allowing for precise identification and quantification of target molecules within tissue sections. High background signal can obscure or falsely amplify the fluorescence from the actual targets, leading to misinterpretation of the data and unreliable conclusions. Any fluorescence signal we find here likely stems from the surface adhering some nucleic acid or proteins that have leached away from tissue. We have found that the surface chemistry is crucial in determining how much background we observe. Having a high background leads to more difficult base-calling and a lower sensitivity, reflected in lower accuracy; therefore, we aim to have as minimal of a background as possible. Additional images were captured between tissue samples to quantify the background.

FIG. 4, bottom, provides a comparison of the background signal related to the surface chemistry of glass slides functionalized with an amine-containing polymer control or a polymer described herein. Aggregates formed at the surface of the glass slide can contribute to the background signal. Using a glass slide functionalized with the amine-containing polymer control, high background signal was observed in lanes probed with antibody-oligo conjugates. Using a glass slide functionalized with poly PEGMA-NH2, background signal was low for lanes probed with antibody-oligo conjugates specific for.

The poly PEGMA-NH2 coating demonstrated significantly lower background signals and more consistent performance across different tissue types, and creates a more stable and reliable adhesive environment, suitable for the rigorous demands of high-throughput in situ workflows.

Transcript detection in tonsil. Tonsil tissues sections were deposited onto the surface of each lane and were processed using deparaffinization and antigen retrieval steps as described supra. Detection of the nucleic acid of interest requires a detection agent, such as an oligonucleotide probe or padlock probe, with a sequence capable of hybridizing with the nucleic acid of interest to facilitate its detection in situ. The determination of the sequence of the oligonucleotide label and its association to the nucleic acid of interest is made a priori, and the oligonucleotide label is capable of being detected by various methods. In embodiments, a padlock probe including an oligonucleotide label is hybridized to a nucleic acid of interest and is subjected to ligation using a ligase (e.g., PBCV-1 DNA ligase; commercially available as SplintR®) to form a circular polynucleotide. The circular polynucleotide is amplified prior to detection to boost its signal for detection. In embodiments, the mode of detection is by sequencing-by-synthesis, where the sequence of the oligonucleotide label is detected and used to associate and identify the nucleic acid of interest in the tissue section following bioinformatic analyses.

FIG. 5 provides an image of transcript detection of a tonsil tissue on a poly PEGMA-NH2 coated surface. From this study, it was evident that the polymer formulation us in the flow cell enabled successful and robust tissue adhesion of tissues while minimizing non-specific and background signals.

What is claimed is:

1. A solid support comprising a polymer and a cell or tissue section attached to the polymer, wherein the polymer comprises a subunit having the formula: 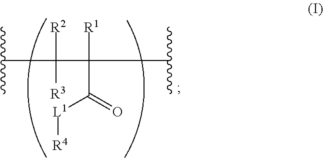
wherein
R¹ is hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is —N₃ or —NR⁵R⁶;
R⁵ is hydrogen, substituted or unsubstituted alkyl, substituted or unsubstituted heteroalkyl, or an amine protecting group;
R⁶ is hydrogen, substituted or unsubstituted alkyl, or substituted or unsubstituted heteroalkyl, or an amine protecting group; and
L¹ is 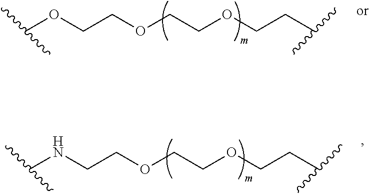
wherein m is an integer from 0 to 24.

2. The solid support of claim 1, wherein R⁴ is —NR⁵R⁶.

3. The solid support of claim 1, wherein R⁴ is —NH₂.

4. The solid support of claim 1, wherein L¹ is 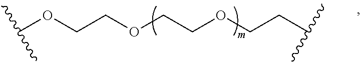
wherein m is an integer from 6 to 24.

5. The solid support of claim 1, wherein L¹ is 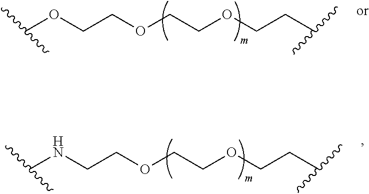
wherein m is an integer from 2 to 12.

6. The solid support of claim 1, wherein L¹ is

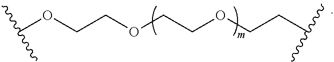

7. The solid support of claim 6, wherein m is an integer from 4 to 12.

8. The solid support of claim 6, wherein m is an integer from 6 to 12.

9. The solid support of claim 1, wherein R² and R³ are hydrogen.

10. The solid support of claim 1, wherein R¹ is hydrogen or substituted or unsubstituted alkyl.

11. The solid support of claim 1, wherein R¹ is unsubstituted alkyl.

12. The solid support of claim 1, wherein the polymer comprises a plurality of subunits having the formula: 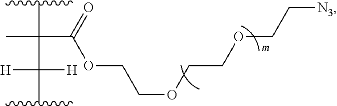
wherein m is an integer from 2 to 24.

13. The solid support of claim 1, wherein the polymer comprises a plurality of subunits having the formula: 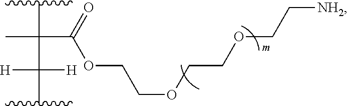
wherein m is an integer from 2 to 24.

14. The solid support of claim 1, further comprising a spacer subunit having the formula 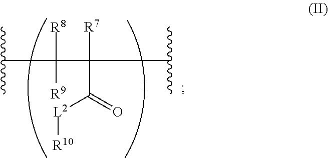
wherein
R⁷ is hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁹ is hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁰ is 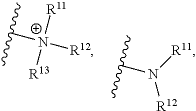
hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹¹ is hydrogen, substituted or unsubstituted alkyl, or substituted or unsubstituted heteroalkyl;
R¹² is hydrogen, substituted or unsubstituted alkyl, or substituted or unsubstituted heteroalkyl;
R¹³ is hydrogen, substituted or unsubstituted alkyl, or substituted or unsubstituted heteroalkyl; and
L² is a covalent linker.

15. The solid support of claim 14, wherein the spacer subunit has the formula:

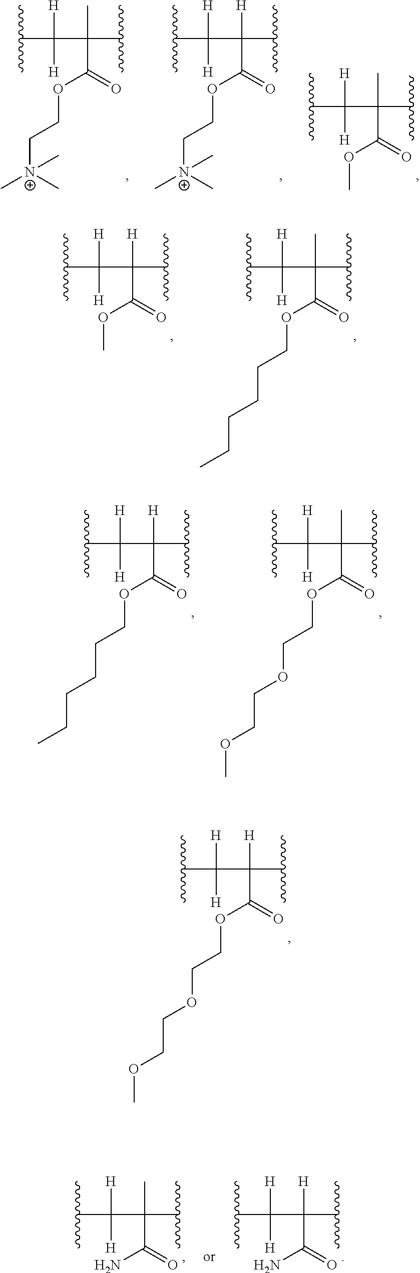

16. The solid support of claim 1, wherein the solid support comprises an IR reflective coating.

17. The solid support of claim 1, further comprising a resist, wherein the resist is between the polymer and the solid support.

18. The solid support of claim 17, wherein the resist is a polydimethylsiloxane (PDMS), poly(methyl methacrylate) (PMMA), cyclic olefin copolymer (COC), silsesquioxane resist, an epoxy-based polymer resist, poly(vinylpyrrolidone-vinyl acrylic acid) copolymer resist, an Off-stoichiometry thiol-enes (OSTE) resist, amorphous fluoropolymer resist, a crystalline fluoropolymer resist, polysiloxane resist, SU-8 resist, or an organically modified ceramic polymer resist.

19. The solid support of claim 17, wherein the resist is an organically modified ceramic polymer resist.

20. A method of making a solid support, the method comprising: binding a first polymer to the solid support and contacting the first polymer with a reducing agent to generate a second polymer, wherein the first polymer comprises polymerized subunits having the formula: 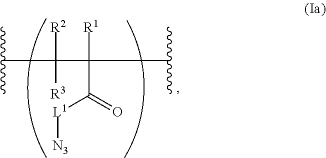
and the second polymer comprises polymerized subunits having the formula: 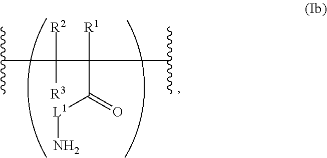
wherein
R¹ is independently hydrogen, substituted or unsubstituted alkyl, or substituted or unsubstituted heteroalkyl;
R² is independently hydrogen or substituted or unsubstituted alkyl;
R³ is independently hydrogen or substituted or unsubstituted alkyl;
L¹ is -L^{1A}-L^{1B}-L^{1C}-L^{1E}-; and
L^{1A}, L^{1B}, L^{1C}, L^{1D}, and L^{1E} are independently a bond, —NH—, —O—, —S—, substituted or unsubstituted alkylene, or substituted or unsubstituted heteroalkylene.
